(19) United States
(12) Patent Application Publication
(10) Pub. No.: US 2023/0148848 A1
(43) Pub. Date: May 18, 2023

(54) MEDICAL SYSTEM AND CANNULATION METHOD

(71) Applicant: OLYMPUS CORPORATION, Tokyo (JP)

(72) Inventors: Kazuhiro GONO, Sagamihara-shi (JP); Atsushi CHIBA, Tokyo (JP); Nao INOUE, Isehara-shi (JP)

(73) Assignee: OLYMPUS CORPORATION, Tokyo (JP)

(21) Appl. No.: 17/989,126

(22) Filed: Nov. 17, 2022

Related U.S. Application Data
(60) Provisional application No. 63/280,716, filed on Nov. 18, 2021.
(60) Provisional application No. 63/294,441, filed on Dec. 29, 2021.
(60) Provisional application No. 63/344,735, filed on May 23, 2022.

(51) Int. Cl. A61B 1/018; A61B 1/00

(52) U.S. Cl. CPC A61B 1/018; A61B 1/00133; A61B 1/005

(57) ABSTRACT

A medical system including: an instrument having a motion that is electrically driven, the motion being forward and backward movement of an insertion section, a bending angle of a bending section of the insertion section, and/or rolling rotation of the insertion section; an operation device performing an operation input of the motion; and a processor. The processor controlling the motion based on the operation input in a motion mode, the motion mode being one of a full auto mode to perform automatic control of the motion and a semi auto mode in which an operator manually controls the motion. When controlling the motion in the semi auto mode, the processor interrupting the semi auto mode by automatic control of the motion to perform intervention, Subsequent to the intervention, the processor switching to one of the full auto mode and the semi auto mode based on a use of the instrument.

### CROSS-REFERENCE TO RELATED APPLICATIONS

This application claims the benefit of priority to U.S. Provisional Application Nos. 63/280,716 filed Nov. 18, 2021; 63/294,441 filed Dec. 29, 2021; 63/344,735 filed May 23, 2022, the entire contents of each of which is incorporated herein by reference.

### BACKGROUND

A technique called endoscopic retrograde cholangiopancreatography (ERCP) has been known that captures an X-ray image or a CT image of biliary duct by inserting a cannula into a biliary duct from a treatment tool channel of an endoscope, injecting a contrast agent from the cannula, and performing X-ray imaging or CT imaging. An example in which a robotic catheter system for performing procedures by remotely operating a catheter system is applied to ERCP is also known.

Known is a medical system that remotely performs electric control of a main body of an endoscope inserted into a body cavity and various types of treatment tools each inserted through a forceps channel. A method of advancing/retreating and opening/closing a basket forceps in accordance with an embedded program is also known.

### SUMMARY

Accordingly, A medical system is provided. The medical system comprising: a medical instrument whose instrument motion during a procedure is electrically driven, the instrument motion being at least one of forward and backward movement of an insertion section, a bending angle of a bending section of the insertion section, and rolling rotation of the insertion section; an operation device configured to perform an operation input of the instrument motion; and a processor comprising hardware. The processor being configured to: control the electrically-driven instrument motion based on the operation input in a motion mode, the motion mode being one of a full auto mode to perform automatic control of the electrically-driven instrument motion and a semi auto mode in which an operator manually controls the instrument motion; while the electrically-driven instrument motion is being controlled in the semi auto mode, interrupt the semi auto mode by automatic control of the electrically-driven instrument motion to perform intervention, interrupt the semi auto mode by automatic control of the electrically-driven instrument motion to perform intervention, and subsequent to the intervention, switch to one of the full auto mode and the semi auto mode based on a determined use of the medical instrument.

The processor can be configured to: switch the motion mode to the full auto mode when the medical instrument is being positioned, and switch the motion mode to the semi auto mode when the medical instrument is being used for treatment after being positioned. The treatment can be a cannulation using the medical instrument.

The medical instrument can comprise an endoscope that electrically drives an endoscopic operation, which is the instrument motion, and captures an endoscope image, and the processor can be configured to perform the automatic control of positioning a distal end section of the endoscope based on the endoscope image in the full auto mode.

When the medical instrument contacts an organ or tissue or when contact between the medical instrument and the organ or the tissue is expected in the semi auto mode, the processor can be configured to perform the intervention by automatic control to avoid the contact.

In the semi auto mode, when the medical instrument is moved to a route different from an insertion route in the procedure in which the semi auto mode is set, the processor can perform the intervention by automatic control to restrict movement of the medical instrument to the route.

The processor can include, as the motion mode, a manual mode in which the intervention by automatic control is not performed, and the processor can switch between the full auto mode, the semi auto mode, and the manual mode in accordance with the procedure.

The processor can set the motion mode to the manual mode when the medical instrument is determined to be inserted into a target.

In each step of the procedure, the medical system can enable setting of correspondence as to which of the full auto mode, the semi auto mode, and the manual mode is set based on input information, and the processor can switch between the full auto mode, the semi auto mode, and the manual mode in accordance with the step of the procedure based on the correspondence.

The procedure can be endoscopic retrograde cholangiopancreatography. The medical instrument can include an endoscope that electrically drives an endoscopic operation, which is the instrument motion, and the processor can be configured to: switch the motion mode to the full auto mode when positioning the endoscope to a papillary portion of duodenum, and switch the motion mode to a semi auto mode when inserting a treatment tool into a biliary duct.

The processor can include, as the motion mode, a manual mode in which the intervention by automatic control is not performed with respect to manual control, and the processor is configured to switch between the full auto mode, the semi auto mode, and the manual mode in accordance with performance of an endoscopic retrograde cholangiopancreatography. The medical instrument can include an endoscope that electrically drives an endoscopic operation, which is the instrument motion, and the processor is configured to switch the motion mode to the manual mode when the endoscope is inserted into a papillary portion of duodenum.

After the processor sets to the full auto mode, the processor can be configured to ignore a manual instruction of the instrument motion from the operation device.

Also provided is a cannulation method using an endoscope that is electrically driven during an endoscopic operation, a motion of the endoscope being at least one of forward and backward movement of an insertion section, a bending angle of a bending section of the insertion section, and rolling rotation of the insertion section, the cannulation method comprising: switching to a full auto mode in which automatic control of the electrically-driven endoscopic operation is performed to control the motion of the endoscope to position the endoscope to a papillary portion of duodenum; switching to a semi auto mode in which an operator manually controls the motion of the endoscope; while the motion of the endoscope is being controlled in the semi auto mode, interrupt the semi auto mode by automatic control of the motion of the endoscope to perform intervention, and subsequent to the intervention, switch to one of the full auto mode and the semi auto mode based on a determined use of the endoscope.

Still further provided is a control apparatus for a medical system comprising a processor comprising hardware. The processor being configured to: control an electrically-driven instrument motion of a medical instrument based on an operation input in a motion mode, the motion mode being one of a full auto mode to perform automatic control of the electrically-driven instrument motion and a semi auto mode in which an operator manually controls the instrument motion; when controlling the electrically-driven instrument motion in the semi auto mode, interrupt the semi auto mode by automatic control of the electrically-driven instrument motion to perform intervention, and subsequent to the intervention, switch to one of the full auto mode and the semi auto mode based on a determined use of the medical instrument.

### BRIEF DESCRIPTION OF THE DRAWINGS

FIG. 1 shows organs and tissues involved in the ERCP procedure.

FIG. 2 shows a flow of the ERCP procedure.

FIG. 3 shows a basic configuration example of a medical system.

FIG. 4 shows the flow of ERCP procedure using a medical system.

FIG. 5 shows the vicinity of the distal end of an endoscope positioned by an overtube and a balloon.

FIG. 6 shows configuration examples of an endoscope and a treatment tool when a force sensor is provided.

FIG. 7 shows a first flow of motion mode switching.

FIG. 8 shows a second flow of motion mode switching.

FIG. 9 shows an example of a combination of each step and motion mode.

FIG. 10 shows an example of controlling line-of-sight direction of camera by rolling rotation.

FIG. 11 shows an example of automatic control of a raising base.

FIG. 12 shows an example of automatic insertion of guide wire.

FIG. 13 shows a first detailed configuration example of a medical system.

FIG. 14 shows a detailed configuration example of a drive control device.

FIG. 15 is a schematic view of an endoscope including a bending section and a driving mechanism thereof.

FIG. 16 shows a detailed configuration example of a forward/backward drive device.

FIG. 17 is a perspective view of a connecting section including a rolling drive device.

FIG. 18 shows a detailed configuration example of a distal end section of an endoscope including a raising base of a treatment tool.

FIG. 19 shows a detailed configuration example of a non-electric treatment tool.

FIG. 20 shows a second detailed configuration example of a medical system.

FIG. 21 shows a detailed configuration example of an electric treatment tool.

FIG. 22 is a diagram schematically illustrating organs and tissues that are related to manipulation of endoscopic retrograde cholangiopancreatography (ERCP).

FIG. 23 illustrates the flow of manipulation of the ERCP.

FIG. 24 is a diagram for describing a configuration example of a medical system.

FIG. 25 is a diagram for describing a more detailed configuration example of the medical system.

FIG. 26 is a diagram for describing a configuration example of a drive control device.

FIG. 27 is a diagram schematically illustrating an example of an endoscope including a curved portion and a driving mechanism for the curved portion.

FIG. 28 is a diagram for describing an example of an advancing/retreating driving device.

FIG. 29 is a diagram for describing an example of a coupling element including a roll driving device.

FIG. 30 is a diagram for describing an example of a possible operation of a tip portion that that is positioned with respect to the papillary portion.

FIG. 31 is a diagram for describing an example of the tip portion of an endoscope, the tip portion including a raising base of a treatment tool.

FIG. 32 is a diagram for describing an example of the tip portion including a force sensor and the treatment tool.

FIG. 33 is a diagram for describing an example of control of a basket treatment tool.

FIG. 34 is a flowchart describing a processing example in accordance with the present embodiment.

FIG. 35 is a diagram for describing an example of maintaining the treatment tool in the biliary duct.

FIG. 36 is a diagram for describing an example of markers added to the basket treatment tool.

FIG. 37 is another diagram for describing an example of the markers added to the basket treatment tool.

FIG. 38 is a diagram for describing an example of a method of determining that the treatment tool has been inserted.

FIG. 39 is a flowchart describing another processing example in accordance with the present embodiment.

FIG. 40 is a diagram for describing an example of a position maintaining mode.

FIG. 41 is a flowchart describing a processing example of treatment mode control.

FIG. 42 is a flowchart describing a processing example of endoscope control.

FIG. 43 is a diagram for describing an example of controlling a raising base to maintain the treatment tool.

FIG. 44 is a flowchart describing a modification of the treatment mode control.

FIG. 45 is a diagram for describing restriction on operation input.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

### Explanation of ERCP

The present embodiment relates to switching of motion mode when performing ERCP using an electric medical system. ERCP stands for Endoscopic Retrograde Cholangiopancreatography. First, before describing the present embodiment, the details of procedure of ERCP is described below.

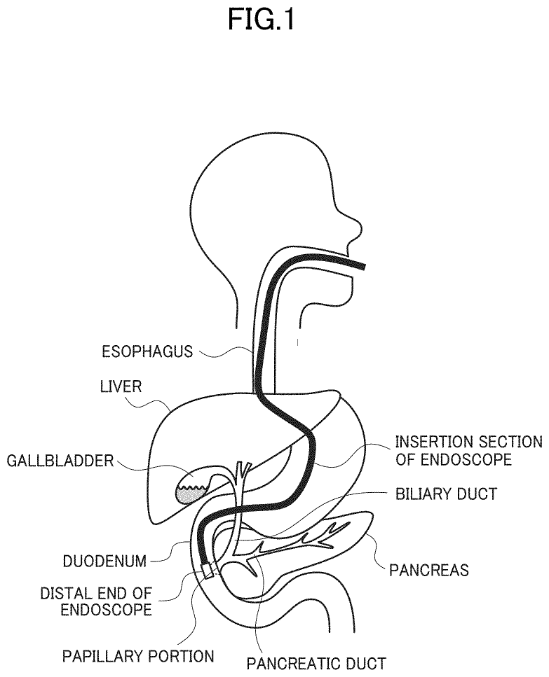

FIG. 1 shows organs and tissues involved in the ERCP procedure. The organs include a multiple types of tissues, forming a unique structure with a specific function. In FIG. 1, the liver, gallbladder, pancreas, esophagus, stomach, and duodenum are shown as organs. Tissues are formed by related cells combined, and examples include blood vessels, muscles, skin, and the like. In FIG. 1, a biliary duct and a pancreatic duct are shown as tissues.

The biliary duct is the target of the ERCP procedure. The biliary duct is a pipeline for allowing the bile produced in the liver to flow into the duodenum. When approaching the biliary duct using an endoscope, a treatment tool inserted into the channel of the endoscope is inserted to the biliary duct from the papillary portion of the duodenum while holding the endoscope at the position of the duodenum. Hereinafter, the papillary portion of the duodenum is simply referred to as a papillary portion. The papillary portion is a region including an opening of the luminal tissue with respect to the duodenum. Not only the opening but also the structure around the opening is referred to as a papillary portion. The opening of the luminal tissue is the opening of a common duct with respect to the duodenum. The common duct is formed as the confluence of the biliary duct and pancreatic duct. However, as described later, the papillary portion largely varies between individuals. For example, in some cases, the biliary duct opens directly to the duodenum without being merged with the pancreatic duct. In this case, the opening of the luminal tissue is the opening of the biliary duct.

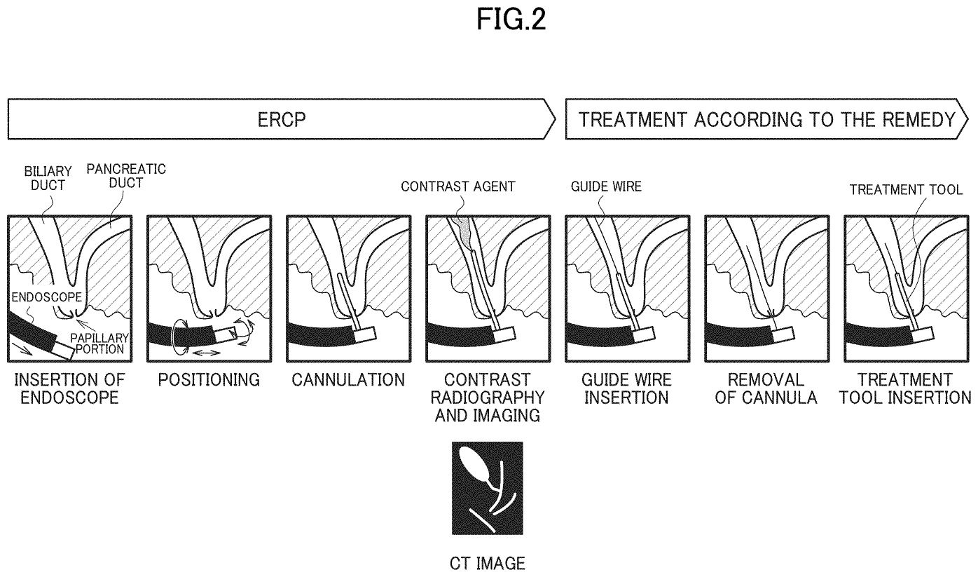

FIG. 2 shows a flow of the ERCP procedure. In ERCP, a side-viewing type endoscope in which a camera, an illumination lens, and an opening of a treatment tool channel are provided on a side surface of a distal end section of the endoscope is used. The camera is also referred to as an imaging device.

In the endoscope insertion step, the insertion section of the endoscope is inserted from the mouth to the duodenum through the esophagus and stomach. At this time, the insertion section is inserted until the papillary portion becomes roughly visible in the field of view of the endoscope. Next, in the positioning step, the position of the endoscope is adjusted relative to the papillary portion. Specifically, the position of the distal end section of the endoscope is adjusted so that the papillary portion is within the imaging range of the camera of the endoscope. Alternatively, the position of the distal end section of the endoscope is adjusted so that the camera of the endoscope is facing directly front of the papillary portion and the papillary portion appears in the center of the field of view.

Then, in the cannulation step, a cannula is inserted from the papillary portion into the biliary duct. Specifically, the cannula is inserted into the treatment tool channel of the endoscope so that the cannula protrudes from the channel opening of the distal end section of the endoscope. The distal end of the cannula is inserted into the common duct from the opening of the common duct, and the cannula is further inserted through the confluence of the biliary duct and the pancreatic duct toward the direction of the biliary duct. Cannulation refers to insertion of a cannula into a body. A cannula is a medical tube that is inserted into a body for medical purposes.

Next, in the contrast radiography and imaging step, a contrast agent is injected into the cannula and poured into the biliary duct through the distal end of the cannula. By performing X-ray or CT imaging in this state, an X-ray image or a CT (Computed Tomography) image showing the biliary duct, gallbladder, and pancreatic duct can be obtained. The procedure of ERCP has been described. After the procedure, various treatments are performed according to the results of diagnosis based on the X-ray image or CT image. An example of the treatment is described below.

In a guide wire insertion step, a guide wire is inserted into a cannula so that the guide wire is protruded from the distal end of the cannula, and the guide wire is inserted into the biliary duct. In a cannula removing step, the cannula is removed while leaving the guide wire inside the biliary duct. As a result, only the guide wire protrudes from the distal end section of the endoscope, indwelling in the biliary duct. Next, in a treatment tool insertion step, the treatment tool is inserted into the biliary duct along the guide wire. An example of a treatment tool is a basket or stent. The basket is used with a catheter. While allowing the guide wire to pass through the catheter, the catheter is inserted into the biliary duct along the guide wire. A basket made of a plurality of metal wires is inserted into the biliary duct from the distal end of the catheter. An object to be removed, such as a gallstone, is placed in the basket and held. Such object to be removed is taken out from the biliary duct by removing the basket and catheter in this state from the biliary duct. A stent is also used in a similar manner with a catheter and inserted into the biliary duct from the distal end of the catheter. The narrow portion of the biliary duct can be widened by inserting a stent; further, by keeping the stent therein, the narrow portion is held in a widened state by the indwelling stent.

The procedure of ERCP is thus performed; however, since, for example, the operator performs the procedure while referring to the endoscope image, or the operator operates the distal end section from the base end side of the long insertion section, etc., there are difficulties in the procedure. For example, in the cannulation step, the operator performs the cannulation by referring to an endoscope image, which shows the papillary portion from the outside. Since there are various individual differences in the form of the papillary portion when viewed from the outside, or there are various individual differences in the paths of the biliary duct and the pancreatic duct, there are difficulties in the procedure of cannulation.

To overcome the difficulties, it is possible to electrically drive the medical system to enable the medical system to perform the ERCP procedure in a full auto mode, thereby assisting the operator. However, during the procedure steps, there are times when the operator desires to manually operate an endoscope or a treatment tool, rather than have the medical system operate the endoscope or the treatment tool in a full auto mode. For example, in some cases, the operator may prefer a full auto mode of the medical system in performing the positioning step, etc. before the cannulation, but desires to manually operate subtle operations in the cannulation step. Although switching of the motion mode can be done manually by button operation or the like, automatic switching may be considered to be more convenient.

### Procedure Flow and Medical System According to the Present Embodiment

Therefore, the present embodiment enables switching of motion mode according to the procedure step in the electrically-driven medical system, thereby assisting the ERCP procedures.

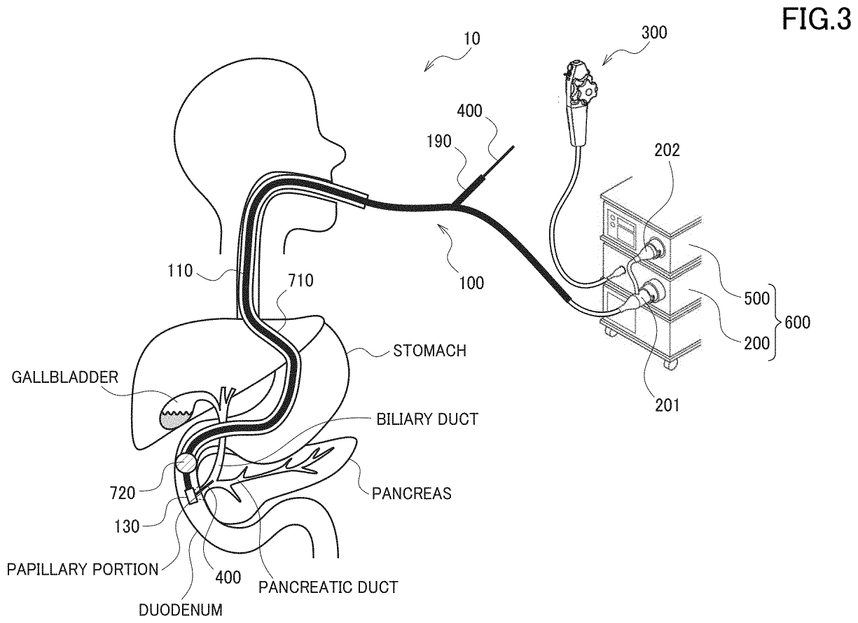

FIG. 3 shows a basic configuration example of a medical system 10 according to the present embodiment. The medical system 10 includes an endoscope 100, an operation device 300, an overtube 710, a balloon 720, a treatment tool 400, and a control device 600. The medical system 10 is also referred to as an endoscope system or an electric endoscope system. In this example, among the endoscope 100 and the treatment tool 400, only the endoscope 100 is electrically driven. It is also possible to use an electrically-driven treatment tool 400. The details of the medical system 10 using an electrically-driven treatment tool 400 are described later.

The overtube 710 is a tube with a variable hardness that covers the insertion section 110 of the endoscope 100. The balloon 720 is provided near the distal end on the outer side of the overtube 710. The operator inserts the endoscope 100 and the overtube 710, which is in a soft state, to the duodenum, inflates the balloon 720 to fix a portion around the distal end of the overtube 710 to the duodenum, and hardens the overtube 710. When the endoscope 100 and the overtube 710 are inserted into the body, at least the bending section of the insertion section 110 is exposed from the distal end of the overtube 710. The bending section refers to a section structured to be bent at an angle corresponding to the bending operation in the vicinity of the distal end of the insertion section 110. The base end of the overtube 710 is present outside the body. The base end side of the insertion section 110 is exposed from the base end of the overtube 710. Note that the example shown here uses the overtube 710 and the balloon 720, but these may be omitted.

An insertion opening 190 of the treatment tool is provided at the base end side of the insertion section 110, and a treatment tool channel for allowing the treatment tool 400 to pass through from the insertion opening 190 to the opening of the distal end section 130 is provided inside the insertion section 110. The insertion opening 190 of the treatment tool is also called a forceps opening; however, the treatment tool to be used is not limited to forceps.

The endoscope 100 is detachably connected to a control device 600 using connectors 201 and 202. The control device 600 includes a drive control device 200 to which the connector 201 is connected, and a video control device 500 to which the connector 202 is connected. The drive control device 200 controls the electrical driving of the endoscope 100 via the connector 201. An operation device 300 for manually operating the electrical driving is connected to the drive control device 200. The video control device 500 receives an image signal from a camera provided at the distal end section 130 of the endoscope 100 via the connector 202, generates a display image from the image signal, and displays it on a display device (not shown). In FIG. 3, the drive control device 200 and the video control device 500 are shown as separate devices, but they may be structured as a single device. In this case, the connectors 201 and 202 may be integrated into a single connector.

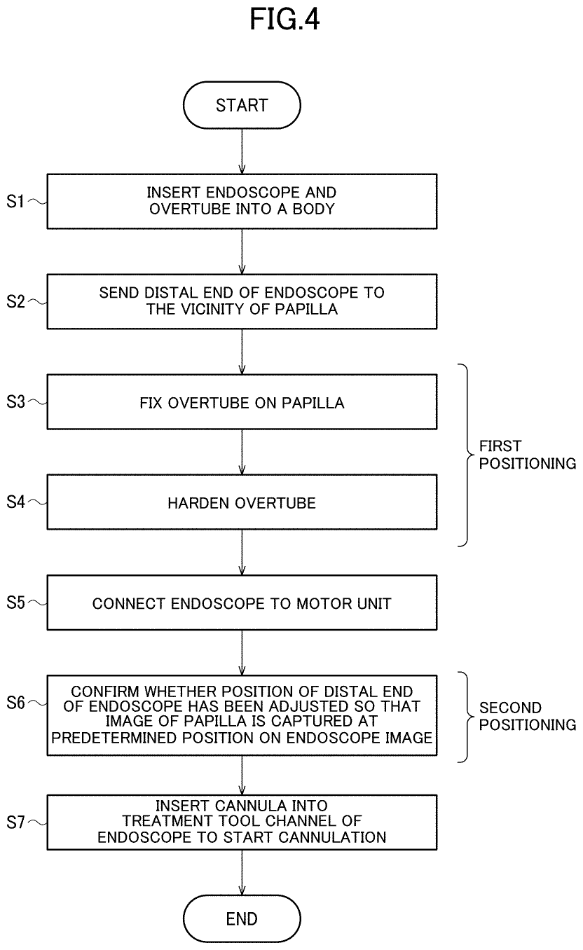

Before describing the motion mode switching of the present embodiment, the flow of ERCP procedure using the medical system 10 is described with reference to FIG. 4.

This example assumes a medical system using an electric medical instrument, which is the endoscope 100 or the treatment tool 400. That is, the forward and backward movement, the bending of the bending section, and the rolling rotation of the insertion section 110 of the endoscope 100 are electrically driven; otherwise, the forward and backward movement, the bending of the bending section, and the rolling rotation of the insertion section of the treatment tool 400 are electrically driven. However, it is sufficient that the instrument motion, which is at least one of these motions, is electrically driven. It is also possible to electrically drive the raising motion of the treatment tool 400. The term “electrical driving” means that medical instrument is driven by a motor or the like based on an electrical signal for controlling the instrument motion. For example, when the electrical driving is manually operated, an operation input to the operation device 300 is converted into an electrical signal, and the medical instrument is driven based on the electrical signal. In the following, the forward and backward movement may be simply referred to as “forward/backward movement”.

In step S1, the operator inserts the insertion section 110 of the endoscope 100 and the overtube 710 into the duodenum. More specifically, in a state where the insertion section 110 is inserted into the overtube, the insertion section 110 and the overtube 710 are inserted into the duodenum together. The overtube 710, which is changeable in hardness, is soft in step S1. For example, the operator can move the insertion section 110 and the overtube 710 forward by a non-electrically-driven manual operation so that they are inserted into the body. The non-electrical driving means that the endoscope 100 is not electrically driven by a motor or the like, instead, the force applied to the operation section is directly transmitted to the endoscope 100 by a wire or the like, thereby operating the endoscope 100. For example, in the present embodiment, steps S1 to S4 are not electrically driven. In this case, it is sufficient that at least the forward/backward movement is not electrically driven, and the bending, the rolling rotation, or both may be manually operated by electrical driving.

In step S2, the operator inserts the insertion section 110 until the distal end section 130 reaches the vicinity of the papillary portion. For example, when the operator manually inserts the insertion section 110 by non-electrical driving, the operator inserts the insertion section 110 until the papillary portion becomes visible in the endoscope image. At this point, the distal end of the endoscope 100 does not need to accurately reach the papillary portion; the distal end of the endoscope 100 may reach a position before the papillary portion or past the papillary portion.

In step S3, the operator fixes the distal end of the overtube 710 to the duodenum. As an example, the operator performs an operation to inflate the balloon 720 provided near the distal end of the overtube 710, and fixes the distal end of the overtube 710 to the duodenum by the balloon 720. In step S4, the operator performs an operation to harden the overtube 710. At this time, the overtube 710 is hardened while maintaining its shape in a state immediately before hardening, that is, the shape when it is inserted from the mouth to the duodenum. As a result, the insertion section 110 is held by the hardened overtube 710 and the balloon 720, thereby fixing the insertion route of the insertion section 110. These steps S3 and S4 are referred to as first positioning.

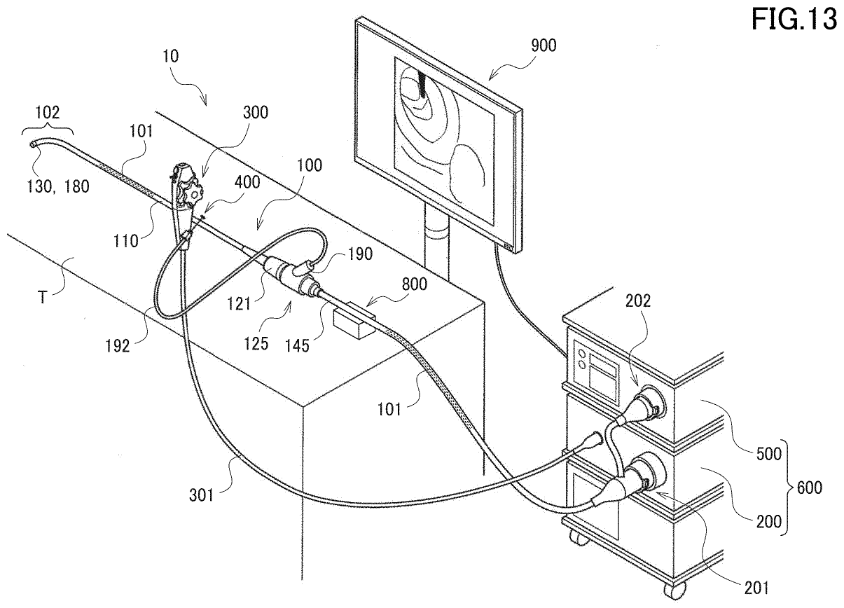

In step S5, the endoscope 100 is connected to the motor unit, and the non-electrical driving is switched to the electrical driving. The method of switching between the non-electrical driving and the electrical driving varies depending on the configuration of the drive mechanism. For example, when the medical system 10, which is described later with reference to FIG. 13, is used, in steps S1 to S4, the forward/backward movement is non-electrically driven and the bending and the rolling rotation are electrically driven. In this case, the forward/backward movement may be switched from the non-electrical driving to the electrical driving by connecting the endoscope 100 to the forward/backward drive device 800. Further, when the bending operation by non-electrical driving is enabled by providing a bending operation dial or the like capable of non-electrically performing the bending operation, the bending movement may be switched from the non-electrical driving to the electrical driving, for example, by connecting the connector 201 to the drive control device 200. Alternatively, even if the motor unit is kept connected, the motor may be structured to be detachable by a clutch mechanism or the like, and the non-electrical driving may be switched to the electrical driving by the clutch mechanism. Step S5 may be performed before step S1. For example, when the forward/backward movement is manually operated by electrical driving, the endoscope 100 may be connected to the motor unit before step S1. In this case, the motion mode is set to the manual mode in steps S1 to S4, and the operator manually operates the electric medical instrument.

In step S6, the motion mode is set to the full auto mode, and the drive control device 200 automatically positions the distal end section 130 at the papillary portion. The operator can then confirm that the position of the distal end section 130 has been adjusted so that the papillary portion is captured at a predetermined position on the endoscope image. The drive control device 200 acquires an endoscope image from the video control device 500 and performs positioning of the distal end section 130 of the endoscope 100 based on the endoscope image. More specifically, the drive control device 200 controls the forward/backward movement, bending, or rolling rotation by electrical driving so that the papillary portion is captured at a position registered in advance on the endoscope image. The position registered in advance is, for example, the center of the image. The positioning may be performed so that the opening of the luminal tissue is captured at a position registered in advance. Further, the drive control device 200 may perform electrical driving control based on the endoscope image so that the camera directly faces the front of the papillary portion or so that the papillary portion is captured at an appropriate angle of view. This step S6 is referred to as second positioning. The processor may restrict an operator's instruction by the operation device during the full auto mode.

In step S7, the operator inserts a cannula into the treatment tool channel through the insertion opening 190 to start cannulation into the biliary duct. As an example, in step S7, when a predetermined condition is satisfied, for example, in a state such that the motion mode is set to the semi auto mode, the operator manually operates the electric medical instrument. When the endoscope or the treatment tool is about to contact an organ, the drive control device 200 causes intervention of the automatic control during the manual operation so as to cause the medical instrument to perform an avoiding action.

The details of the motion mode described above are described later. The correspondence between each step and the motion mode described above is merely an example, and each step and the motion mode may be associated with each other in various ways. For example, the user may be able to set the association between each step and the motion mode as described below.

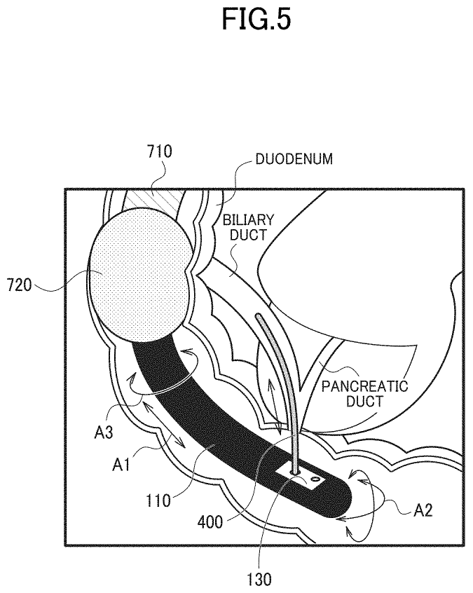

FIG. 5 shows the vicinity of the distal end of an endoscope positioned by the overtube 710 and the balloon 720. As shown in FIG. 5, the balloon 720 is fixed at a position slightly apart from the papillary portion to the pyloric side of the stomach. More specifically, the balloon 720 is positioned closer to the base end of the insertion section 110 than the base end of the bending section of the insertion section 110. By combining such a balloon 720 with the overtube 710 having a variable hardness, the bending section exposed to the papillary portion side from the balloon 720 and the distal end section 130 can be freely operated without being fixed. Thus, the electrical driving from the base end side can be efficiently transmitted to the distal end section 130 of the endoscope.

The endoscopic operation by the electrical driving is the forward and backward movement shown in A1, a bending movement shown in A2, or a rolling rotation shown in A3. The forward movement is a shift toward the distal end side along the axial direction of the insertion section 110, and the backward movement is a shift toward the base end side along the axial direction of the insertion section 110. In the following, the forward and backward movement is also referred to as the forward/backward movement. The bending movement is a movement by which the angle of the distal end section 130 is changed due to the bending of the bending section. The bending movement includes bending movements in two orthogonal directions, which can be controlled independently. One of the two orthogonal directions is referred to as the vertical direction and the other is referred to as the horizontal direction. The rolling rotation is a rotation about an axis of the insertion section 110.

If the treatment tool 400 is electrically driven in addition to the endoscope 100, the electrically-driven treatment tool motion is the forward and backward movement, the bending movement or the rolling rotation. The meanings of these motions are the same as those in the case of the endoscopic operation described above. The adjustment of the raising angle of the treatment tool 400, which protrudes from an opening on the side surface of the distal end section 130, may be electrically driven.

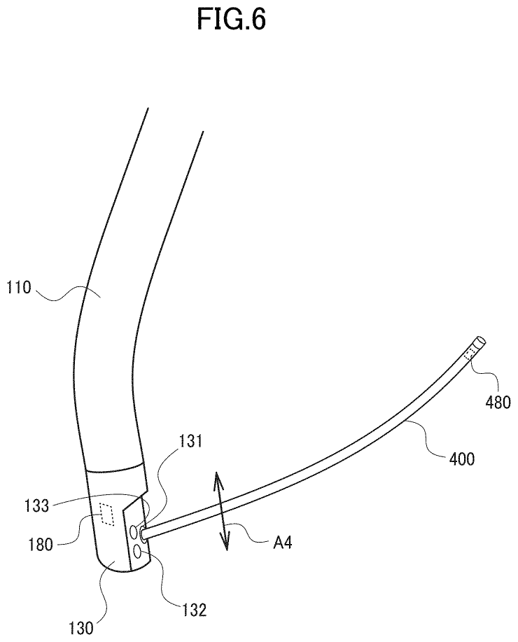

FIG. 6 shows configuration examples of the endoscope 100 and the treatment tool 400 when a force sensor is provided. This is an example of contact detection in the semi auto mode. However, if contact detection is performed by other means, such as images, as described later, the force sensor may not be provided.

The endoscope 100 includes a force sensor 180 provided at the distal end section 130. The distal end section 130 is covered with a rigid housing, such as a metal or plastic housing, to which the force sensor 180 is fixed. An example of the force sensor 180 is a strain gauge.

A strain gage is a mechanical sensor that measures the strain of a housing. The housing is deformed as the distal end section 130 comes in contact with organs, and the like, and the strain gauge detects the deformation, thereby detecting the stress of the contact. The strain gage has a thin insulator and a metal foil resistor provided on the insulator. When stress is applied to an object to which the strain gage is attached, the strain gage is deformed together with the object, and the resistance of the metal foil resistor is changed by the deformation. The drive control device 200 measures this resistance, thereby detecting the stress.

A force sensor 480 is fixed in the same way to the distal end section of the treatment tool 400. Although an example in which the force sensor is provided in both the endoscope 100 and the treatment tool 400 is shown herein, the force sensor may be provided in only one of the endoscope 100 and the treatment tool 400. Further, although an example in which one force sensor is provided for each of the endoscope 100 and the treatment tool 400 is shown herein, a plurality of force sensors may be provided in the endoscope 100, or a plurality of force sensors may be provided in the treatment tool 400.

### Switching of Motion Mode

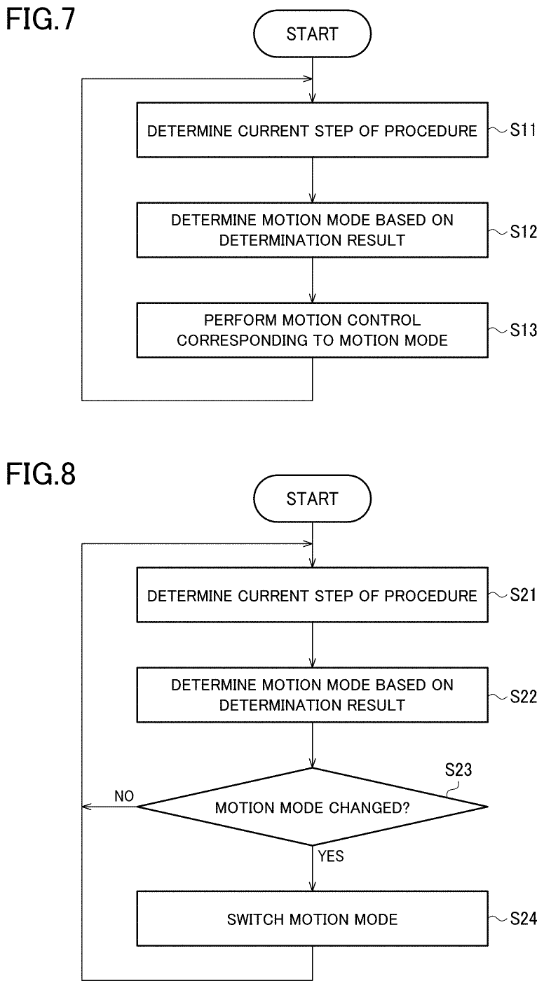

FIG. 7 shows a first flow of motion mode switching. In step S11, the drive control device 200 determines which step of the procedure is being performed. In step S12, the drive control device 200 determines the motion mode based on the determination result. That is, each step is associated with the motion mode in advance, and the drive control device 200 refers to the correspondence to determine the motion mode corresponding to the step of the determination result. In step S13, the drive control device 200 controls the endoscopic operation or the treatment tool motion in the motion mode thus determined. Thereafter, steps S11 to S13 are repeated.

If it is determined in step S11 that the procedure of a step different from the previous step is being performed, and in step S12, a motion mode different from the previously-set motion mode is determined, the motion mode is switched. That is, the motion mode is switched according to the procedure.

FIG. 8 shows a second flow of motion mode switching. Steps S21 and S22 are similar to steps S11 and S12 in FIG. 7. In step S23, the drive control device 200 determines whether or not the motion mode thus determined in step S22 is different from the previous motion mode. If a motion mode different from the previous motion mode is determined, the drive control device 200 switches to the determined motion mode in step S24. If the motion mode same as the previous motion mode is determined, the drive control device 200 maintains the motion mode and the sequence returns to step S21.

The flows in FIGS. 7 and 8 are merely examples, and any processing flow can be used as long as the motion mode can be switched according to the procedure step.

The details of the motion mode are described below. The motion mode specifies how the electrically-driven instrument motion is controlled, and includes a full auto mode, a semi auto mode and a manual mode. Note that at least two of these modes may be switchable; for example, it may be arranged such that switching is performed between the full auto mode and the semi auto mode, and the manual mode is omitted.

In the full auto mode, the drive control device 200 automatically controls the electrically-driven instrument motion. The automatic control means that not the operator but the drive control device 200 makes a decision to control the instrument motion, i.e., the drive control device 200 controls the instrument motion according to a predetermined control algorithm. In the full auto mode, the drive control device 200 does not accept manual operation by the operator or disables it.

For example, the drive control device 200 performs automatic control of the instrument motion based on the result of a process using machine learning. Specifically, the storage section in the drive control device 200 stores a trained model. The trained model is trained so that it receives an input of an endoscope image and outputs the corresponding instructions of instrument motion using teacher data in which endoscope images are associated with correct data indicating the instrument motion to be performed at that time. The drive control device 200 inputs an endoscope image to the trained model, which in response performs inference of corresponding instruction of instrument motion. The drive control device 200 then controls the instrument motion according to the instruction of instrument motion.

The means for performing the full auto mode is not limited to machine learning. For example, in the case of automatic insertion of an endoscope, the route to the destination and its motion may be programmed in advance, and the drive control device 200 may execute the program to automatically control the instrument motion.

In the semi auto mode, the drive control device 200 controls the instrument motion according to manual operation by the operator; however, under predetermined conditions, automatic control of instrument motion intervenes. The predetermined condition is a condition in which the treatment tool and an organ or a tissue have a specific relationship. When the predetermined condition is satisfied, the drive control device 200 causes intervention of the automatic control so as to avoid or cancel the specific relationship. For example, a predetermined condition that the medical instrument comes in contact with an organ or a tissue, or that the medical instrument is likely to contact an organ or a tissue. In this case, the drive control device 200 causes intervention of the automatic control so as to avoid contact between the medical instrument and the organ or the tissue. For example, when the distal end section of an endoscope or a treatment tool is about to contact the intestinal wall of the duodenum, the contact is automatically avoided. Alternatively, the predetermined condition is that the medical instrument moves to a route different from the insertion route in the procedure step. In this case, the drive control device 200 causes intervention of automatic control to restrict the movement toward the route different from the insertion route in the procedure step, or intervention of automatic control to place it back to the insertion route in the procedure step. For example, during cannulation to the biliary duct, when the cannula moves forward from the common duct toward the pancreatic duct, the move is automatically restricted.

As shown in FIG. 3, it may be arranged such that, instead of using an electric endoscope, the endoscopic operation is electrically driven by operating a non-electric endoscope by a robotic arm. In such a case, the operator would manually operate the robot arm so as to operate the endoscope via the robotic arm; in this case, there is a possibility of contact between the robotic arm moving outside the body and an inspection device or the like. The inspection device may be, for example, a C-arm of an X-ray inspection system. If there is such a contact outside the body, the drive control device 200 may perform the automatic control of the robotic arm to avoid the contact.

Upon the manual operation in the semi auto mode, all of the instrument motions of the electrically-driven instrument motions may be manually operated, or some the instrument motions may be automatically controlled. For example, it may be arranged such that the forward/backward movement of the endoscope is manually operated and the bending movement of the endoscope is automatically controlled.

Upon the intervention of automatic control in the semi auto mode, the intervention of the automatic control may be performed with respect to some or all of the electrically-driven instrument motions. For example, it may be arranged such that the bending movement of the endoscope is automatically controlled to avoid collision, while there is no intervention of automatic control for the forward/backward movement and the rolling rotation.

Further, when the medical instrument is performing an instrument motion and a predetermined condition is satisfied, the intervention of automatic control may be performed for the instrument motion being currently performed, the intervention of automatic control may be performed for another instrument motion, or the intervention of automatic control may be performed for all of the instrument motions. For example, when the endoscope is curved and collides with an organ, the collision may be avoided by automatically controlling the bending movement or by automatically controlling the forward/backward movement, the bending movement, and the rolling rotation.

The contact can be detected using the force sensors 180 and 480 described above. To predict the contact, for example, a distance measuring sensor such as a TOF sensor may be provided at the distal end section of the endoscope 100 or the treatment tool 400. The drive control device 200 may then determine the possibility of the contact when the distance measured by the distance measuring sensor is equal to or less than the threshold. Alternatively, the drive control device 200 may predict the contact by a process of recognition of the image and the operation information. The recognition process may be an image recognition process using machine learning. The storage section of the drive control device 200 stores a trained model. The trained model is trained so that it receives an endoscope image and an operation input and outputs the corresponding possibility of contact using teacher data in which the endoscope image and the operation input are associated with correct data indicating the possibility of contact at that time. The drive control device 200 inputs an endoscope image to the trained model, which in response performs inference of corresponding possibility of contact.

The determination of the insertion route is performed, for example, by a recognition process of image and operation information. For example, a recognition process using machine learning may be used. The storage section of the drive control device 200 stores a trained model. The trained model is trained so that it receives an endoscope image and an operation input and outputs the corresponding insertion route using teacher data in which the endoscope image and the operation input are associated with correct data indicating the insertion route of the endoscope or the treatment tool at that time. The drive control device 200 inputs an endoscope image and an operation input to the trained model, which in response performs inference of the insertion route of the endoscope or the treatment tool.

In the manual mode, the operator operates the operation device 300 and the drive control device 200 controls the electrically-driven instrument motion according to the operation input. In the manual mode, the drive control device 200 does not allow intervention of the automatic control during the manual operations.

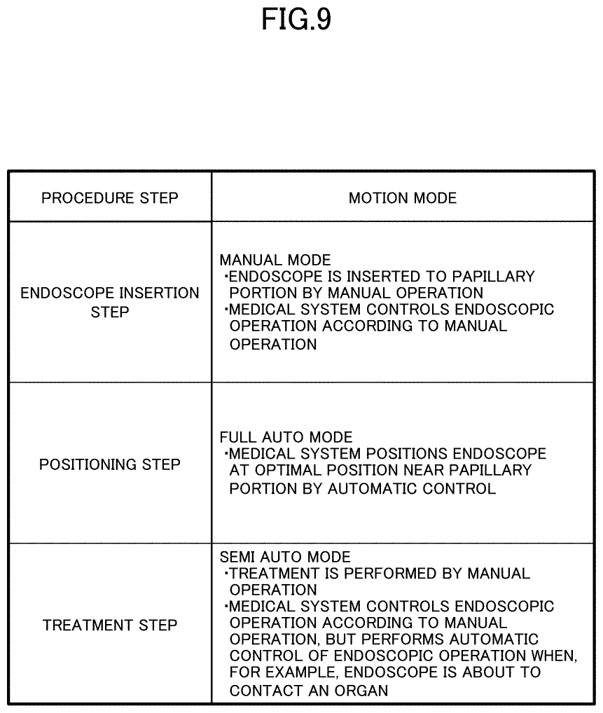

FIG. 9 shows an example of a combination of each step and motion mode. However, the combination of each step and motion mode is not limited to those shown in FIG. 9.

The endoscope insertion step corresponds to the endoscope insertion step in FIG. 2 or steps S1 and S2 in FIG. 4. In the endoscope insertion step, the drive control device 200 sets the manual mode as the motion mode. The operator inserts the endoscope to the papillary portion by manual operation. The drive control device 200 controls the electrically-driven endoscopic operation according to the manual operation.

The positioning step corresponds the positioning step in FIG. 2 or step S6 in FIG. 4. In the positioning step, the drive control device 200 sets the full auto mode as the motion mode, and places the distal end section 130 of the endoscope 100 to the optimal position with respect to the papillary portion by the automatic control.

The treatment step corresponds to one of the steps after the cannulation step in FIG. 2 or step S7 in FIG. 4. In the treatment step, the drive control device 200 sets the semi auto mode as the motion mode. The operator operates the endoscope by manual operation. The drive control device 200 controls the endoscopic operation according to the manual operation; however, when the endoscope 100 is about to contact an organ, the drive control device 200 performs automatic control of the endoscopic operation to move it in a direction in which the contact can be avoided.

Next, the method of determining the procedure step in S11 of FIG. 7 or S21 of FIG. 8 is described below.

In the first example, the drive control device 200 determines the procedure step by an image recognition process using machine learning. The storage section of the drive control device 200 stores a trained model. The trained model is trained so that it receives an input of an endoscope image and outputs the corresponding procedure step using teacher data in which endoscope images are associated with correct data indicating the procedure step at that time. The drive control device 200 inputs an endoscope image to the trained model, which in response performs inference of corresponding procedure step. For example, when the distal end section 130 of the endoscope 100 is positioned at the stomach or the duodenum, which is before the papillary portion, it is determined that the procedure step is the endoscope insertion step. When the distal end section 130 of the endoscope 100 is positioned at the papillary portion and the treatment tool 400 is not shown, it is determined that the procedure step is the positioning step. When the distal end section 130 of the endoscope 100 is positioned at the papillary portion and the treatment tool 400 is shown, it is determined that the procedure step is the treatment step.

In the second example, the drive control device 200 determines the procedure step based on the position of the endoscope 100 detected by UPD, or the like. UPD is an endoscope insertion shape observation device. The insertion shape of the endoscope is detected by detecting magnetism from the coils provided at predetermined intervals along the longitudinal direction of the insertion section 110 by an observation device to thereby detect the position of each coil, and connecting the positions of the coils. The drive control device 200 determines the position of the distal end section 130 of the endoscope 100 based on its insertion shape and determines the procedure step based on the position.

In the third example, the drive control device 200 determines the content of the operation based on the operation input to the operation device 300 and determines the procedure step based on the content of the operation. For example, if the forward/backward movement of the endoscope is being performed, it is determined that the procedure step is the endoscope insertion step. If the forward/backward movement of the treatment tool is being performed, or if the endoscope and the treatment tool are being operated, it is determined that the procedure step is the treatment step.

In FIG. 9, each step is associated with one of the motion modes; however, each step may be associated with any of the motion modes. For example, it may be arranged such that the operator inputs information of correspondence between each step and a motion step, and the drive control device 200 sets the motion step based on the correspondence information externally input. Alternatively, the drive control device 200 may set the motion step based on the operator's profile information. For example, the drive control device 200 stores the correspondence between each step and the motion mode in the case where the operator is an experienced operator. as the drive control device 200 further stores the correspondence between each step and the motion mode in the case where the operator is less experienced. The control device 200 then selects an appropriate correspondence depending on whether the operator is experienced or less experienced based on the operator's profile information.

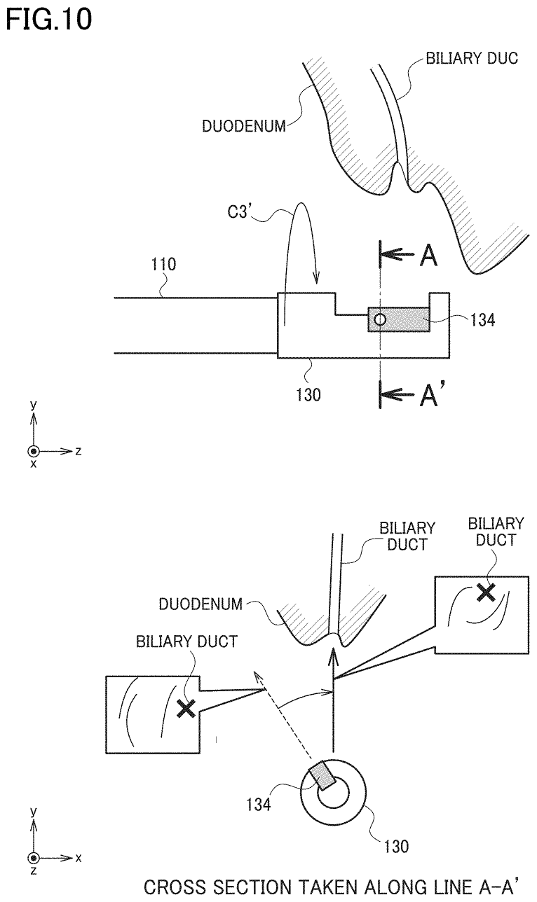

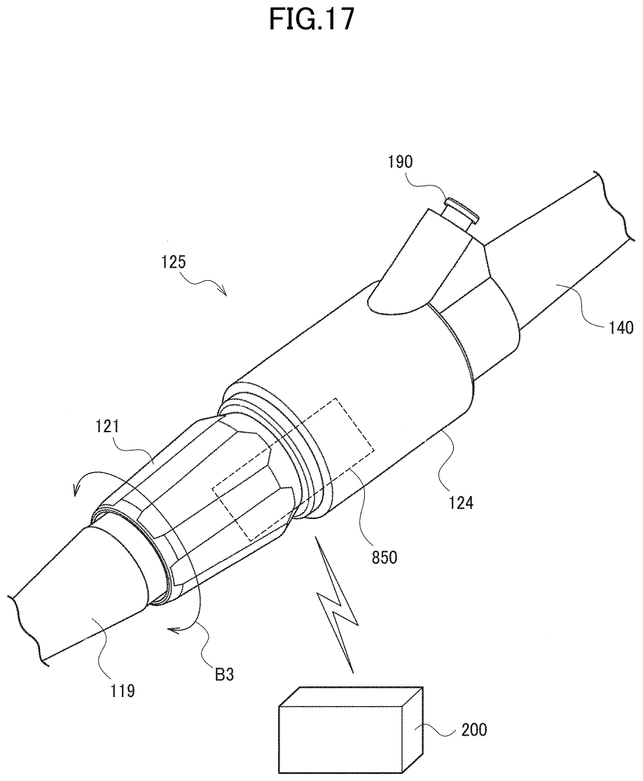

FIG. 10 shows an example of controlling line-of-sight direction of camera by rolling rotation. As an example of the rolling rotation, as described later with reference to FIG. 17, the base end section of the insertion section 110 is caused to undergo rolling rotation, thereby causing the distal end section 130 of the insertion section 110 to undergo rolling rotation. FIG. 10 shows another example in which the distal end section 130 is caused to undergo rolling rotation, instead of causing the base end section of the insertion section 110 to undergo rolling rotation.

As shown in C3′ in the upper figure of FIG. 10, the distal end section 130 undergoes rolling rotation with respect to the insertion section 110 of the endoscope about the rotation axis in the axial direction. For example, a mechanism for converting wire traction to the rolling rotation of the distal end section 130 is provided in the distal end section 130, and the drive control device 200 drives the wire traction, thereby electrically driving the rolling rotation of the distal end section 130. The axial direction of the distal end section 130 is referred to as z, and the two directions orthogonal to the z direction are referred to as x and y. The lower figure of FIG. 10 shows a cross-sectional view of the upper figure in the z-direction, taken along line A-A′. When the distal end section 130 undergoes the rolling rotation, the line-of-sight direction of the camera rotates in the xy-plane, and the position of the opening of the biliary duct in the image moves.

This method is used, for example, in the positioning step in the full auto mode. Alternatively, it may be used in the treatment step in the full auto mode. With the rolling rotation, the camera of the endoscope is made to directly face the papillary portion. The line-of-sight direction of the camera can also be changed by bending, and the camera of the endoscope may be made to directly face the papillary portion by the rolling rotation and bending, or only by bending.

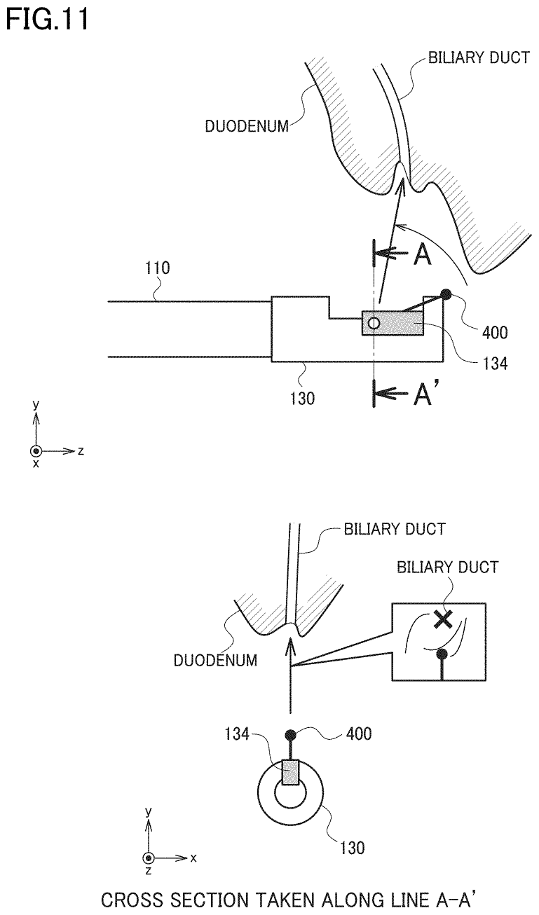

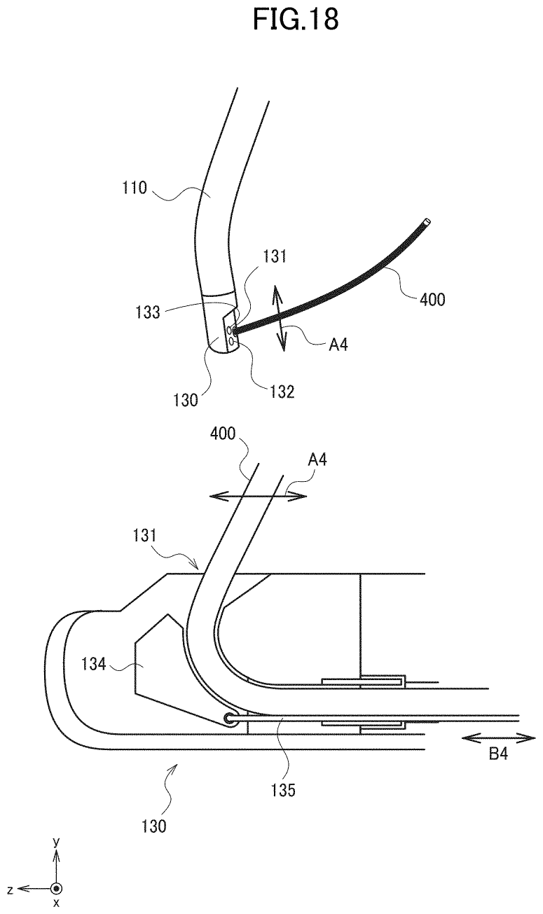

FIG. 11 shows an example of automatic control of a raising base. The raising base 134 is a mechanism that changes the direction of the treatment tool 400 that protrudes from the distal end section 130 of the endoscope 100. The electric mechanism of the raising base 134 is described later with reference to FIG. 18. As shown in the upper figure of FIG. 11, the drive control device 200 controls the raising angle of the treatment tool 400 by rotating the raising base 134 about the rotation axis orthogonal to the axial direction of the distal end section 130. The lower figure of FIG. 11 shows a cross-sectional view of the upper figure in the z-direction, taken along line A-A′. As shown in the upper and lower figures, the drive control device 200 automatically controls the raising angle of the treatment tool 400 according to the travelling direction of the biliary duct. For example, the drive control device 200 calculates the travelling direction of the biliary duct based on the endoscope image or a CT image, and lifts up the raising base 134 so that the relationship between the travelling direction of the biliary duct thus calculated and the raising angle of the treatment tool 400 becomes appropriate.

This method is used, for example, in the treatment step in the full auto mode. In this case, the method of FIG. 10 and the method of FIG. 11 may be combined.

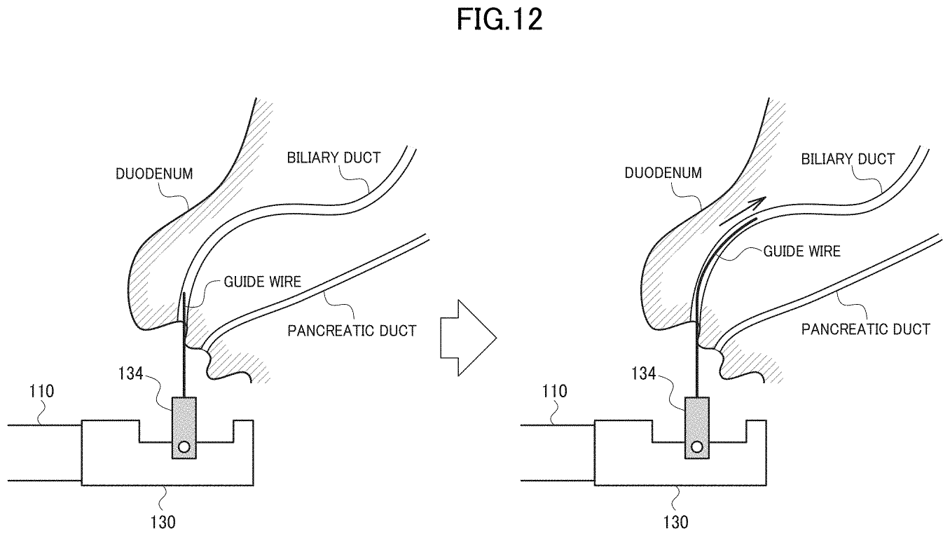

FIG. 12 shows an example of automatic insertion of guide wire. This method inserts the guide wire directly into the biliary duct without using a cannula. The drive control device 200 automatically inserts the guide wire based on the shape of the biliary duct. For example, the drive control device 200 presumes the shape of the biliary duct based on the result of MRCP (Magnetic Resonance Cholangio Pancreatography) upon the insertion of the guide wire, and automatically bends or moves the guide wire forward/backward along the shape of the biliary duct. For example, the guide wire may be electrically driven using a method similar to the electrical driving of the endoscope or the treatment tool.

This method is used, for example, in the treatment step in the semi auto mode. For example, when the guide wire is forced to be bent by an excessive power, the drive control device 200 causes intervention of the automatic control during the manual operation of the guide wire, thereby avoiding exertion of a large force to the biliary duct.

### First Detailed Configuration Example of Medical System

FIG. 13 shows a first detailed configuration example of the medical system 10. In this configuration example, among the endoscope and the treatment tool, the endoscope is electrically driven. The medical system 10 is a system for observing or treating the inside of the body of a patient lying on an operating table T. The medical system 10 includes an endoscope 100, a control device 600, an operation device 300, a treatment tool 400, a forward/backward drive device 800, and a display device 900. The control device 600 includes a drive control device 200 and a video control device 500.

The endoscope 100 is a device to be inserted into a lumen of a patient for the observation of an affected part. In this embodiment, the side to be inserted into a lumen of a patient is referred to as “distal end side” and the side to be attached to the control device 600 is referred to as “base end side”. The endoscope 100 includes an insertion section 110, a connecting section 125, an extracorporeal soft section 145, and connectors 201 and 202. The insertion section 110, the connecting section 125, the extracorporeal soft section 145, and the connectors 201 and 202 are connected one another in this order from the distal end side.

The insertion section 110 is a portion to be inserted into a lumen of a patient, and is configured in a soft elongated shape. The insertion section 110 includes a bending section 102, an extracorporeal soft section for connecting the base end of the bending section 102 and the connecting section 125, and a distal end section 130 provided at the distal end of the bending section 102. An internal route 101 is provided inside the insertion section 110, the connecting section 125, and the extracorporeal soft section 145, and a bending wire passing through the internal route 101 is connected to the bending section 102. When the drive control device 200 drives the wire via the connector 201, the bending section 102 curves. Further, a raising base wire connected to the raising base provided at the distal end section 130 is connected to the connector 201 through the internal route 101. As the drive control device 200 drives the raising base wire, the raising angle of the treatment tool 400 protruding from the side surface of the distal end section 130 is changed. The side surface of the distal end section 130 is provided with a camera, an illumination lens, and an opening of a treatment tool channel. An image signal line for connecting the camera and the connector 202 is provided in the internal route 101, and an image signal is transmitted from the camera to the video control device 500 via the image signal line. The video control device 500 displays an endoscope image generated from the image signal on the display device 900. As described in FIG. 6, the force sensor 180 is provided in the housing of the distal end section 130. A signal line for connecting the force sensor 180 and the connector 201 is provided in the internal route 101, and a force detection signal is transmitted from the force sensor 180 to the drive control device 200 via the signal line.

The connecting section 125 is provided with an insertion opening 190 of the treatment tool and a rolling operation section 121. The treatment tool channel is provided in the internal route 101, one end of which is open to the distal end section 130 and the other end of which is open to the insertion opening 190 of the treatment tool. An extension tube 192 extending from the insertion opening 190 to the operation device 300 is connected to the insertion opening 190. The treatment tool 400 is inserted from an opening on the operation device 300 side of the extension tube 192, and protrudes to the opening of the distal end section 130 via the insertion opening 190 and the treatment tool channel. The extension tube 192 may be omitted, and the treatment tool 400 may be inserted through the insertion opening 190. The rolling operation section 121 is attached to the connecting section 125 so as to be rotatable about the axial direction of the insertion section 110. By rotating the rolling operation section 121, the insertion section 110 undergoes rolling rotation. As described later, the rolling operation section 121 can be electrically driven.

The forward/backward drive device 800 is a drive device for moving the insertion section 110 forward and backward by electrical driving. An extracorporeal soft section 140 is detachable from the forward/backward drive device 800, and an insertion section 110 moves forward and backward when the forward/backward drive device 800 causes the extracorporeal soft section 140 to slide in the axial direction in a state in which the extracorporeal soft section 140 is mounted on the forward/backward drive device 800. Although FIG. 13 shows an example in which the extracorporeal soft section 140 and the forward/backward drive device 800 are detachable, there is no such limitation, and it may be arranged such that the connecting section 125 and the forward/backward drive device 800 are detachable.

The operation device 300 is detachably connected to the drive control device 200 via an operation cable 301. The operation device 300 may communicate with the drive control device 200 through wireless communication instead of wired communication. When an operator operates the operation device 300, a signal of the operation input is transmitted to the drive control device 200 via the operation cable 301, and the drive control device 200 electrically drives the endoscope 100 to enable an endoscopic operation corresponding to the operation input based on the signal of the operation input. The operation device 300 has an operation input section having five or more channels corresponding to the forward and backward movement of the endoscope 100, the bending movements in two directions and the rolling rotation, and the operation of the raising base. If one or more of these operations are not electrically driven, the operation input section may be omitted. Each operation input section includes, for example, a dial, a joystick, a D-pad, a button, a switch, a touch panel, and the like.

The drive control device 200 electrically drives the endoscope 100 by driving a built-in motor based on an operation input to the operation device 300. Alternatively, when the motor is present outside the drive control device 200, the drive control device 200 transmits a control signal to the external motor based on an operation input to the operation device 300, thereby controlling the electrical driving. In addition, the drive control device 200 may drive a built-in pump or the like based on an operation input to the operation device 300, thereby causing the endoscope 100 to perform air supply suction. The air supply suction are performed through an air supply/suction tube provided in the internal route 101. One end of the air supply/suction tube opens to the distal end section 130 of the endoscope 100, while the other end is connected to the drive control device 200 via the connector 201. In addition, the treatment tool channel may be extended to the connector 201, and the treatment tool channel may also be used as an air supply/suction tube.

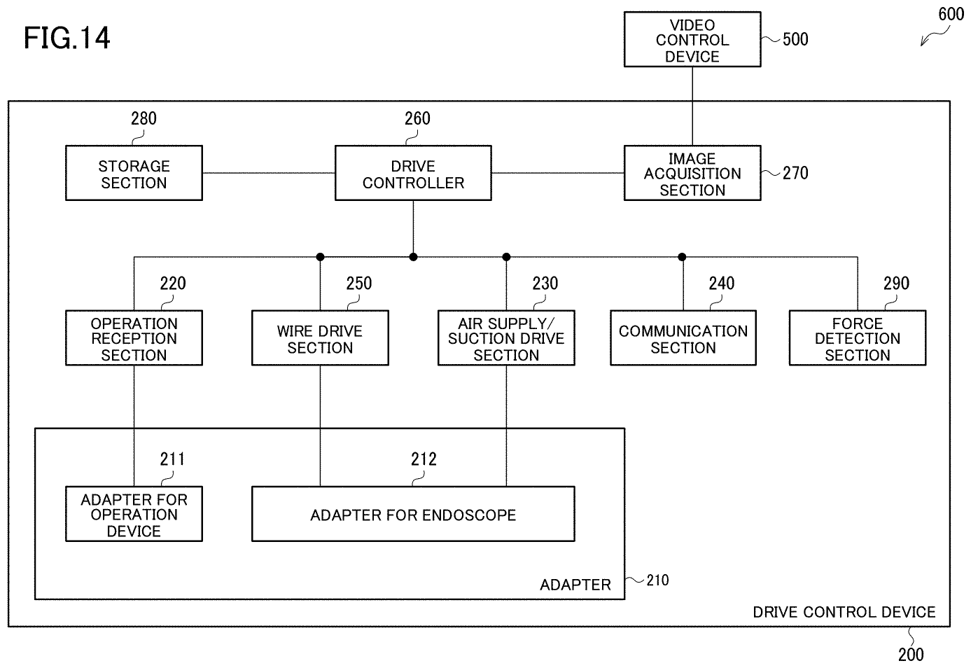

FIG. 14 shows a detailed configuration example of a drive control device 200. The drive control device 200 includes a storage section 280, a drive controller 260, an operation reception section 220, a wire drive section 250, an air supply/suction drive section 230, a communication section 240, and an adapter 210. Further, the drive control device 200 may include an image acquisition section 270 and a force detection section 290.

The adapter 210 includes an operation device adapter 211 to which the operation cable 301 is detachably connected, and an endoscope adapter 212 to which the connector 201 of the endoscope 100 is detachably connected.

The wire drive section 250 drives the bending movement of the bending section 102 of the endoscope 100 or the operation of the raising base of the treatment tool 400 based on the control signal from the drive controller 260. The wire drive section 250 includes a bending movement motor unit for driving the bending section 102 of the endoscope 100 and a raising base motor unit for driving the raising base. The endoscope adapter 212 has a bending movement coupling mechanism for enabling coupling to the bending wire on the endoscope 100 side. When the bending movement motor unit drives the coupling mechanism, the driving force is transmitted to the bending wire on the endoscope 100 side. Further, the endoscope adapter 212 has a raising base coupling mechanism for enabling coupling to the raising base wire on the endoscope 100 side. When the raising base motor unit drives the coupling mechanism, the driving force is transmitted to the raising base wire on the endoscope 100 side.

The air supply/suction drive section 230 drives air supply suction of the endoscope 100 based on a control signal from the drive controller 260. The air supply/suction drive section 230 is connected to an air supply/suction tube of the endoscope 100 via the endoscope adapter 212. The air supply/suction drive section 230 includes a pump or the like, and supplies air to the air supply/suction tube or sucks air from the air supply/suction tube 172.

The communication section 240 communicates with a drive device provided outside the drive control device 200. The communication may be wireless communication or wired communication. The drive device provided outside is a forward/backward drive device 800 for performing forward and backward movement, a rolling drive device for performing the rolling rotation or the like.

The drive controller 260 controls the forward and backward movement, the bending movement and the rolling rotation of the endoscope 100, the raising angle of the treatment tool 400 made by the raising base, and the air supply suction by the endoscope 100. The drive controller 260 is, for example, a processor such as a CPU (Central Processing Unit), a GPU (Graphics Processing Unit), a DSP (Digital Signal Processor), or the like. For example, the storage section 280 stores a computer-readable program, and the functions of the drive controller 260 are implemented as processes as the processor executes the program. The storage section 280 is a storage device such as a semiconductor memory or a magnetic storage device. The semiconductor memory may be a volatile memory such as a SRAM or a DRAM, or a nonvolatile memory such as an EEPROM. However, the hardware of the drive controller 260 is not limited to that described above, and may be structured using circuits with various configurations.

The electrical control performed by the drive controller 260 includes the manual mode, the semi auto mode, and the full auto mode, as described above. As described in FIG. 7, FIG. 8 or FIG. 9, the drive controller 260 switches the motion mode according to the procedure step.

The following describes the control upon the motion mode switching. The image acquisition section 270 is a communication interface for receiving image data of an endoscope image from the video control device 500 by wired communication or wireless communication. The drive controller 260 determines the procedure step based on the endoscope image from the image acquisition section 270 and the operation input signal from the operation reception section 220, and sets a motion mode according to the procedure step. If the machine learning is used, the trained model is stored in the storage section 280. Alternatively, it may be arranged such that an UPD is connected to the drive control device 200, and the drive controller 260 obtains information of endoscope shape from the UPD, determines the procedure step based on the endoscope shape information, and set a motion mode according to the procedure step.

Next, the control in the manual mode is described below. The operation reception section 220 receives an operation input signal from the operation device 300 via the operation cable 301 attached to the operation device adapter 221. When the operation device 300 communicates with the drive control device 200 by wireless communication, the operation reception section 220 may be a wireless communication circuit.

The drive controller 260 controls the electrical driving based on an operation input signal from the operation reception section 220. Specifically, when the bending operation is performed, the drive controller 260 outputs a control signal indicating the bending direction or the bending angle to the wire drive section 250, and the wire drive section 250 drives the bending wire so that the bending section 102 curves in the bending direction or the bending angle. Also, when the forward and backward movement operation is performed, the drive controller 260 transmits a control signal indicating the forward/backward direction or the forward/backward movement amount to the forward/backward drive device via the communication section 240. The forward/backward drive device then moves the extracorporeal soft section 140 forward or backward so that the endoscope 100 moves forward or backward in the forward/backward direction or the forward/backward movement amount. Further, when the rolling rotation operation is performed, the drive controller 260 transmits a control signal indicating the rolling rotation direction or the rolling rotation angle to the rolling drive device via the communication section 240. The rolling drive device then performs rolling rotation of the insertion section 110 so that the endoscope 100 undergoes rolling rotation in the rolling rotation direction or at the rolling rotation angle. Similar controls are performed for other electrical driving.

Next, the control in the semi auto mode is described below. The manual operation in the absence of intervention of automatic control is similar to the manual mode. Next, intervention of automatic control is described below.

The force detection section 290 detects the stress applied to the distal end of the treatment tool or the endoscope from the output signals of the force sensors 180 and 480. The force detection section 290 includes, for example, an amplifier circuit that amplifies the output signal of the force sensor, and an A/D converter that performs A/D conversion of the output signal of the amplifier circuit and outputs the stress detection data to the drive controller 260. The drive controller 260 performs contact determination based on the stress detection data. Alternatively, the drive controller 260 determines possibility of contact or the insertion route based on the endoscope image from the image acquisition section 270 and the operation input signal from the operation reception section 220. If the machine learning is used, the trained model is stored in the storage section 280. When the drive controller 260 determines that a predetermined condition, such as contact, is satisfied, the drive controller 260 causes intervention of automatic control during the manual operation, so as to avoid the contact.

Next, the control in the full auto mode is described. The drive controller 260 performs automatic control of the endoscope or the treatment tool based on the endoscope image from the image acquisition section 270. Alternatively, it may be arranged such that the drive controller 260 obtains an endoscope image and a CT image or MRCP image, and performs the automatic control of the endoscope or the treatment tool based on the images. If the machine learning is used, the trained model is stored in the storage section 280.

In embodiments where the endoscope image is not used for the contact detection or the procedure step determination, the image acquisition section 270 may be omitted. Similarly, in embodiments where the force sensor is not used for the contact detection, the force detection section 290 may be omitted.

### Detailed Configuration Example of Each Part of Medical System

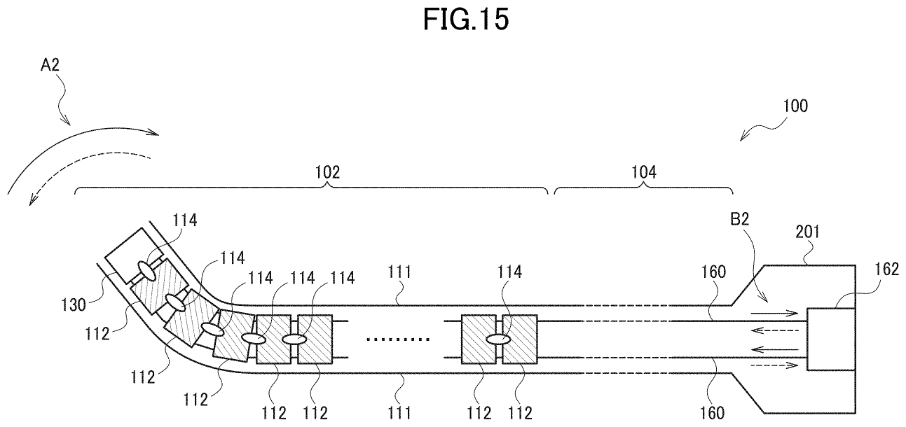

FIG. 15 is a schematic view of an endoscope 100 including a bending section 102 and a driving mechanism thereof. An endoscope 100 includes a bending section 102, a soft section 104, and a connector 201. The soft section 104 corresponds to the intracorporeal soft section and the extracorporeal soft section 145 described above with reference to FIG. 13. In FIG. 15, the connecting section 125 is omitted.

The bending section 102 and the soft section 104 are covered with an outer sheath 111. The inside of the tube of the outer sheath 111 corresponds to the internal route 101 in FIG. 13. The bending section 102 includes a plurality of bending pieces 112 and a distal end section 130 connected to the distal end of the bending pieces 112. Each of the plurality of bending pieces 112 and the distal end section 130 is connected in series from the base end side to the distal end side by a rotatable connecting section 114, thereby forming a multi joint structure. The connector 201 is provided with a coupling mechanism 162 on the endoscope side connected to a coupling mechanism on the drive control device 200 side. By attaching the connector 201 to the drive control device 200, it is possible to electrically drive the bending movement. A bending wire 160 is provided in the outer sheath 111. One end of the bending wire 160 is connected to the distal end section 130. The bending wire 160 passes through the soft section 104 by penetrating through a plurality of bending pieces 112, turns back in a coupling mechanism 162, passes through the soft section 104 again, penetrates through the plurality of bending pieces 112. The other end of the bending wire 160 is connected to the distal end section 130. The driving force from the wire drive section 250 is transmitted to the bending wire 160 via the coupling mechanism 162 as the pulling force of the bending wire 160.

As shown by the solid line arrow B2, when the upper wire in the figure is pulled, the lower wire is pushed, whereby the multiple joints of the bending pieces 112 are bent upward in the figure. As a result, as indicated by the solid line arrow A2, the bending section 102 is curved upward in the figure. When the lower wire in the figure is pulled as indicated by the dotted arrow B2, similarly, the bending section 102 is curved downward in the figure as indicated by the dotted arrow A2. As described with reference to FIG. 5, the bending section 102 can be curved independently in two orthogonal directions. Although FIG. 15 shows a bending mechanism for one direction, two sets of bending wires are actually provided, and each bending wire can be curved independently in two directions by being pulled independently by the coupling mechanism 162.

Note that the mechanism for the electrically-driven bending is not limited to that described above. For example, a motor unit may be provided instead of the coupling mechanism 162. Specifically, it may be arranged such that the drive control device 200 transmits a control signal to the motor unit via the connector 201, and the motor unit drives the bending movement by pulling or relaxing the bending wire 160 based on the control signal.

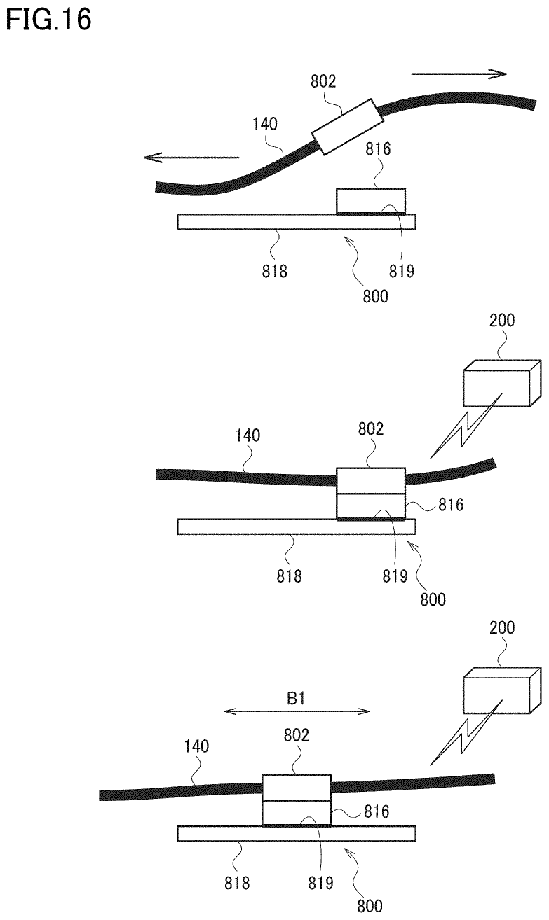

FIG. 16 shows a detailed configuration example of a forward/backward drive device 800. The forward/backward drive device 800 includes a motor unit 816, a base 818, and a slider 819.

As shown in the upper and middle figures, the extracorporeal soft section 140 of the endoscope 100 is provided with an attachment 802 detachable from the motor unit 816. As shown in the middle figure, the attachment of the attachment 802 to the motor unit 816 enables electrical driving of forward/backward movement. As shown in the lower figure, the slider 819 supports the motor unit 816 while enabling the motor unit 816 to move linearly with respect to the base 818. The slider 819 is fixed to the operating table T shown in FIG. 13. As shown in B1, the drive control device 200 transmits a forward or backward control signal to the motor unit 816 by wireless communication, and the motor unit 816 and the attachment 802 move linearly on the slider 819 based on the control signal. As a result, the forward and backward movement of the endoscope 100 shown in A1 in FIG. 5 is achieved. Note that the drive control device 200 and the motor unit 816 may be connected by wired connection.

FIG. 17 is a perspective view of the connecting section 125 including a rolling drive device 850. The connecting section 125 includes a connecting section main body 124 and a rolling drive device 850.

The insertion opening 190 of the treatment tool is provided in the connecting section main body 124 and is connected to the treatment tool channel inside the connecting section main body 124. The connecting section main body 124 has a cylindrical shape, and a cylindrical member coaxial with the cylinder is rotatably provided inside the connecting section main body 124. The base end section of the intracorporeal soft section 119 is fixed to the outside of the cylindrical member, and the base end section serves as a rolling operation section 121. As a result, the intracorporeal soft section 119 and the cylindrical member can rotate with respect to the connecting section main body 124 about the axial direction of the intracorporeal soft section 119. The rolling drive device 850 is a motor unit provided inside the connecting section main body 124. As shown in B3, the drive control device 200 transmits a rolling rotation control signal to the rolling drive device 850 by wireless communication, and the rolling drive device 850 rotates the base end section of the intracorporeal soft section 119 with respect to the connecting section main body 124 based on the control signal, thereby causing rolling rotation of the intracorporeal soft section 119. As a result, the rolling rotation of the endoscope 100 shown in A3 in FIG. 5 is achieved. The rolling drive device 850 may include a clutch mechanism, and the rolling rotation may be switched between non-electrical driving and electrical driving by the clutch mechanism. The drive control device 200 and the rolling drive device 850 may be connected by wired connection via a signal line passing through the internal route 101.

FIG. 18 shows a detailed configuration example of a distal end section 130 of an endoscope including a raising base of a treatment tool. The upper figure shows an external view of the distal end section 130. An opening 131 of a treatment tool channel, a camera 132, and an illumination lens 133 are provided on the side surface of the distal end section 130. As shown in the lower figure, the direction parallel to the axial direction of the distal end section 130 is defined as z direction, the direction parallel to the line-of-sight direction of the camera 132 is defined as y direction, and the direction orthogonal to the z direction and the y direction is defined as x direction. The lower figure shows a cross-sectional view of the distal end section 130 in a plane that is parallel to the yz plane of the treatment tool channel and that passes through the opening 131 of the treatment tool channel.

The distal end section 130 includes a raising base 134 and a raising base wire 135. The raising base 134 is swingable about an axis parallel to the x direction. One end of the raising base wire 135 is connected to the raising base 134, while the other end is connected to the drive control device 200 via the connector 201. As shown in B4, the wire drive section 250 of the drive control device 200 pushes and pulls the raising base wire 135 to swing the raising base 134, thereby, as shown in A4, changing the raising angle of the treatment tool 400. The raising angle is an angle of the treatment tool 400 protruding from the opening 131. The raising angle can be defined, for example, by an angle formed by the treatment tool 400 protruding from the opening 131 and the z direction.

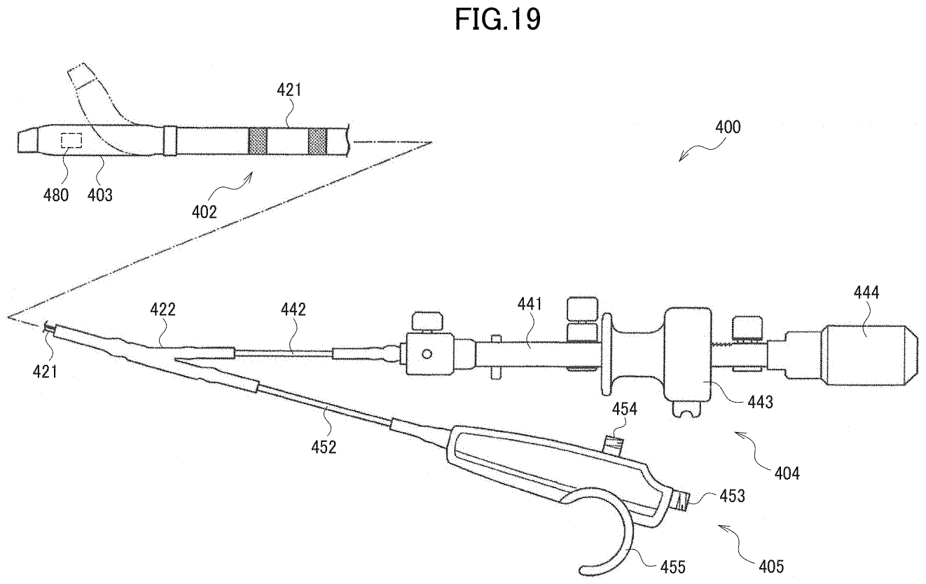

FIG. 19 shows a detailed configuration example of the non-electrically-driven treatment tool 400. Herein, as an example of the treatment tool 400, a cannula capable of operating bending of the distal end is shown. The treatment tool 400 includes a long-length insertion section 402 extending in the axial direction, a bending movement section 403 capable of bending movement, a first operation section 404 for operating the bending movement section 403, and a second operation section 405 for inserting a contrast agent or a guide wire.

The insertion section 402 has a tube 421, and the bending movement section 403 is connected to the distal end of the tube 421. The force sensor 480 described in FIG. 6 is provided near the distal end of the bending movement section 403. The force sensor 480 is provided in areas not involved in the shape change by the bending movement. In FIG. 19, the distal end side of the tube 421 is enlarged. The tube 421 is also referred to as a sheath. The operator holds the tube 421 of the treatment tool 400 inserted into the treatment tool channel of the endoscope 100, and pushes and pulls the tube 421 to move the treatment tool 400 forward and backward.

A connector 422 is connected to the base end of the tube 421. The first operation section 404 and the second operation section 405 are connected to the connector 422. The first operation section 404 includes a connecting tube 442, one end of which is connected to the connector 422, a first operation main body 441 connected to the other end of the connecting tube 442, a grip 444 fixed to the base end of the first operation main body 441, and a slider 443 provided movable forward and backward in the axial direction of the first operation main body 441. Inside the tube 421, the connector 422, the connecting tube 442, and the first operation main body 441, a wire for connecting the bending movement section 423 and the slider 443 is provided. When the operator pulls the slider 443 while holding the grip 444, the wire is pulled and the bending movement section 423 is curved.

The second operation section 405 includes a connecting tube 452, one end of which is connected to the connector 422, a second operation main body 451 connected to the other end of the connecting tube 452, a first opening 453 opened in the axial direction of the connecting tube 452 on the base end side of the second operation main body, a second opening 454 opened to the outer surface of the second operation main body 451, and a hook 455 provided on the second operation main body 451. The hook 455 has elasticity and is formed in a substantially C-shape, and is used for locking the treatment tool 400 to the endoscope 100 or the like. The first opening 453 and the second opening 454 are connected to the tube 421 via the second operation main body 451, the connecting tube 452, and the connector 422. By inserting a contrast agent or a guide wire from the first opening 453 or the second opening 454, the contrast agent can be injected into the body or the guide wire can be inserted into the body from the distal end of the treatment tool 400.

### Second Detailed Configuration Example of Medical System

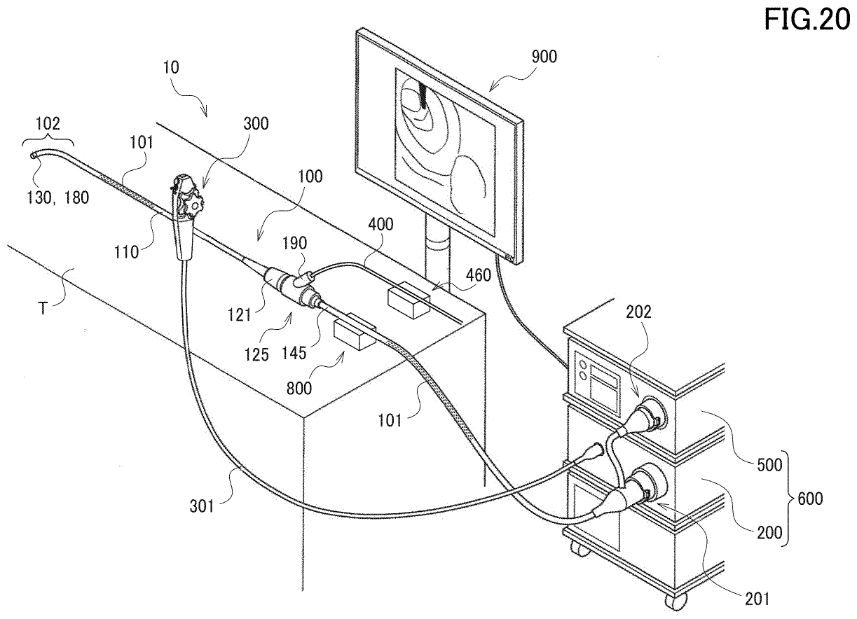

FIG. 20 shows a second detailed configuration example of the medical system 10. In this configuration example, the endoscope and the treatment tool are electrically driven. The following mainly describes structures different from the first detailed configuration example. The medical system 10 includes a forward/backward drive device 460 for a treatment tool.

The treatment tool 400 is inserted from an insertion opening 190 of the connecting section 125, and protrudes to the opening of the distal end section 130 via the treatment tool channel. In this the configuration example, the extension tube 192 of the first detailed configuration example is omitted. The forward/backward drive device 460 is a drive device for moving the treatment tool 400 forward and backward by electrical driving. The tube 421 of the insertion section 402 is detachable from the forward/backward drive device 800, and the insertion section 402 moves forward and backward when the forward/backward drive device 460 slides the tube 421 in the axial direction in a state in which the tube 421 is mounted on the forward/backward drive device 460. The operation device 300 includes an operation input section having eight or more channels corresponding to the forward and backward movement of the endoscope, the bending movements in two directions and the rolling rotation of the endoscope, the movement of the raising base, the forward and backward movement of the treatment tool, and the bending movements in one direction and the rolling rotation of the treatment tool. If one or more of these operations are not electrically driven, the operation input section may be omitted.

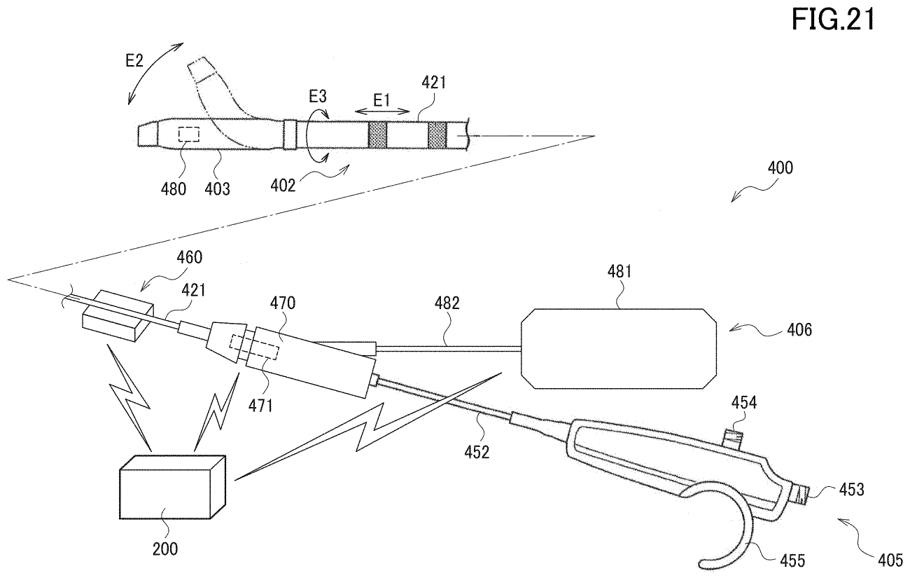

FIG. 21 shows a detailed configuration example of an electric treatment tool 400. The treatment tool 400 includes an insertion section 402, a bending movement section 403, a bending driving section 406 for electrically driving the bending movement section 403, and an operation section 405. Although the reference number 405 is referred to as the second operation section in FIG. 19, 405 is herein referred to as an operation section. The following mainly describes structures different from FIG. 19.

The tube 421 of the insertion section 402 is detachable from the forward/backward drive device 460, and has a structure similar to, for example, the forward/backward drive device 800 for use in endoscopes, which has been described with reference to FIG. 16. That is, the tube 421 has an attachment detachable from the forward/backward drive device 460, and the attachment is attached to the motor unit of the forward/backward drive device 460, thereby enabling the forward/backward movement to be electrically driven. The drive control device 200 transmits a forward or backward control signal to the motor unit by wireless communication, and the motor unit and the attachment move linearly in the axial direction of the tube 421 based on the control signal, thereby moving the insertion section 402 forward or backward. The drive control device 200 and the forward/backward drive device 460 may be connected by wired connection.

A connector 470 is connected to the base end of the tube 421. The bending driving section 406 and the operation section 405 are connected to the connector 470. The connector 470 includes a connecting tube 482, one end of which is connected to the connector 470, and a motor unit 481 connected to the other end of the connecting tube 482. Inside the tube 421, the connector 470, and the connecting tube 482, a wire for connecting the bending movement section 403 and the motor unit 481 is provided. The drive control device 200 transmits a bending movement control signal to the motor unit 481 by wireless communication, and the motor unit 481 drives the wire based on the control signal, thereby bending the bending movement section 403. For example, the electrical driving of the bending movement section 403 can be realized by a structure similar to the structure having a plurality of bending pieces described in FIG. 15. However, although the bending movement of the endoscope is capable of bending in four directions (up, down, left, right), the treatment tool in FIG. 21 is capable of, for example, bending in one direction. Note that the drive control device 200 and the motor unit 481 may be connected by wired connection.

Inside the connector 470, a motor unit 471 is provided to electrically drive the rolling rotation of the insertion section 402. The structures of the connector 470 and the motor unit 471 are similar to, for example, those of the connecting section 125 and the motor unit of the rolling drive device 850 in FIG. 17. Specifically, the connector main body of the connector 470 has a cylindrical shape, and a cylindrical member coaxial with the cylinder is rotatably provided inside the connector main body. The base end section of the tube 421 of the insertion section 402 is fixed to the outside of the cylindrical member. As a result, the tube 421 and the cylindrical member are rotatable with respect to the connector main body about the axial direction of the tube 421. The drive control device 200 transmits a rolling rotation control signal to the motor unit 471 by wireless communication, and the motor unit 471 rotates the base end section of the tube 421 with respect to the connector main body based on the control signal, thereby allowing the insertion section 402 to undergo rolling rotation. Note that the drive control device 200 and the motor unit 471 may be connected by wired connection.

As described above, there are difficulties in the procedure of ERCP since, for example, the operator performs the procedure while referring to the endoscope image, or the operator operates the distal end section from the base end side of the long insertion section, etc. To overcome the difficulties, it is possible to electrically drive the medical system to enable the medical system to perform the ERCP procedure in a full auto mode, thereby assisting the operator. However, during the procedure steps, there are times when the operator desires to manually operate an endoscope or a treatment tool, rather than have the medical system operate the endoscope or the treatment tool in a full auto mode. For this reason, it is desirable to use various motion modes by switching them depending on the procedure step. The U.S. Patent Application Publication No. 2017/0086929 described above discloses an example in which a robotic catheter system is applied to ERCP, but does not disclose or suggest any of the above-mentioned problems or subject matter for solving them.

Therefore, the medical system 10 of the present embodiment includes the medical instrument whose instrument motion is electrically controlled, the operation device 300 that performs an operation input of the instrument motion, and the control device 600 that controls the electrically-driven instrument motion based on the operation input. The instrument motion is at least one of forward and backward movement of an insertion section, a bending angle of a bending section of the insertion section, and rolling rotation of the insertion section. The control device 600 has, as a motion mode, a full auto mode to perform automatic control of the electrically-driven instrument motion, and a semi auto mode to perform intervention by automatic control during manual control in which the electrically-driven instrument motion is controlled based on the operation input. The control device 600 switches between the full auto mode and the semi auto mode according to the step of a procedure using the medical instrument.

According to the present embodiment, switching between the full auto mode and the semi auto mode is performed according to the step of a procedure using the medical instrument. This allows the full auto mode and the semi auto mode, which is essentially a manual operation, to be used separately depending on the procedure step. In addition, performing the intervention by automatic control during the manual control in the semi auto mode enables the health system to temporarily perform automatic control of the medical instrument whenever necessary. For example, even though the ERCP procedure has the above-mentioned difficulties, by temporarily causing intervention by automatic control during the manual operations, it is possible to assist, for example, an inexperienced operator, etc.

The medical instrument and the instrument motion are described in “Procedure Flow and Medical System According to The Present Embodiment”, etc. The full auto mode, the semi auto mode, the switching of the motion mode according to the procedure step are described in “Switching of Motion Mode”, etc.

Further, in the present embodiment, the control device 600 may set the full auto mode as the motion mode in the positioning step of positioning the medical instrument. The control device 600 may set the semi auto mode as the motion mode in the treatment step using the medical instrument after the positioning step. The treatment step may be a cannulation step using the medical instrument.

In the treatment step, the manual operation may be used in some cases. For example, there are cases where the operator wishes to perform the treatment as desired, or the operator wishes to perform the operation according to the situation, such as a situation where the treatment tool must pass through a narrow portion, etc. On the other hand, in the positioning step, the medical instrument is positioned to a predetermined position; therefore, the full auto mode may be used if considered to be more convenient. According to the present embodiment, it is possible to set an appropriate motion mode for these procedure steps.

The setting to the full auto mode in the positioning step and the setting to the semi auto mode in the treatment step are described in “Switching of Motion Mode”, etc.

Further, in the present embodiment, the medical instrument may include the endoscope 100. The endoscope 100 may be such that the endoscopic operation, which is an instrument motion, is electrically driven to capture an endoscope image. The control device 600 may perform the automatic control of positioning the distal end section 130 of the endoscope 100 based on the endoscope image in the full auto mode.

The present embodiment enables full auto control based on the endoscope image. Specifically, by performing the automatic control of the endoscopic operation based on the endoscope image, the endoscopic position is automatically set to a position so that a predetermined organ or tissue etc. is shown at a predetermined position on the endoscope image.

The positioning by the full auto mode is described in the description regarding the positioning step in “Switching of Motion Mode”, step S6 in FIG. 6 in “Procedure Flow and Medical System According to The Present Embodiment”, etc.

Further, in the present embodiment, in the semi auto mode, when the medical instrument contacts an organ or tissue or when contact between the medical instrument and the organ or the tissue is expected, the control device 600 may perform intervention by automatic control to avoid the contact.

According to the present embodiment, when the medical instrument contacts with an organ or tissue, it is possible to avoid the contact. Also, when contact between the medical instrument and the organ or the tissue is expected, it is possible to avoid the contact before it actually occurs, or avoid the strong force exerted to the organ or the tissue due to the contact.

The detection of contact, the prediction of contact, and the avoidance of contact in the semi auto mode are described in “1.3. Switching of Motion Mode”, etc.

Further, in the present embodiment, in the semi auto mode, when the medical instrument is moved to a route different from the insertion route in the step of the procedure in which the semi auto mode is set, the control device 600 performs the intervention by automatic control so as to restrict movement of the medical instrument to the route.

According to the present embodiment, when the medical instrument is moved to a route different from the insertion route in the step of the procedure, it is possible to restrict the movement of the medical instrument to the route. For example, when the medical instrument is inserted into a wrong route, further insertion is restricted. Also, if the medical instrument is about to be inserted into a wrong route, the insertion is restricted and therefore avoided.

The route detection and the restriction of the movement of the medical instrument in the semi auto mode are described in “Switching of Motion Mode”, etc.

Further, in the present embodiment, the control device 600 may include, as a motion mode, a manual mode in which the intervention by automatic control is not performed with respect to the manual control. The control device 600 may switch between the full auto mode, the semi auto mode, and the manual mode according to the step of the procedure. In the step of inserting the medical instrument, the control device 600 may set the manual mode as the motion mode.

Depending on the contents of the procedure, the operator may desire to perform all operations by himself/herself. According to the present embodiment, in the procedure step in which the manual mode is set, the operator can perform all operations by himself/herself without the intervention by the automatic control.

The manual mode and the switching between the full auto mode, the semi auto mode, and the manual mode according to the procedure step are described in “Switching of Motion Mode”, etc.

Further, the present embodiment may enable, in each step of the procedure, setting of correspondence as to which of the full auto mode, the semi auto mode, and the manual mode is set based on input information. The control device 600 may switch between the full auto mode, the semi auto mode, and the manual mode according to the step of the procedure based on the correspondence.

The present embodiment allows the user to set the correspondence between each step and the motion mode by himself/herself. Since the motion mode is selected according to the procedure step based on such correspondence, the procedure can be performed in the user-designed motion mode.

Such external setting of correspondence between each step and the motion mode is described in the description of FIG. 9 in “Switching of Motion Mode”, etc.

Further, in the present embodiment, the procedure may be the endoscopic retrograde cholangiopancreatography.

According to the present embodiment, the motion mode can be switched between the full auto mode and the semi auto mode, or between the full auto mode, the semi auto mode, and the manual mode, according to the procedure step of ERCP.

The ERCP procedure steps are described in “Explanation of ERCP” or FIG. 5 in “Procedure Flow and Medical System According to The Present Embodiment”, etc.

Further, in the present embodiment, the medical instrument may include the endoscope 100 in which the endoscopic operation, which is the instrument motion, is electrically driven. The control device 600 may set the motion mode to the full auto mode in the positioning step of positioning the endoscope 100 to a papillary portion of duodenum. The control device 600 may set the motion mode to the semi auto mode in the cannulation step of inserting the treatment tool 400 into a biliary duct.

In the cannulation step, the manual operation can be used in some cases. For example, there are cases where the operator wishes to perform the treatment as desired, or the operator wishes to perform the operation according to the situation, such as a situation where the treatment tool must pass through a narrow portion, etc. However, automatic control is still desirable in order to avoid exertion of strong force to the intestinal wall, the biliary duct, etc., or to avoid insertion of the treatment tool into the pancreatic duct. On the other hand, in the positioning step, the endoscope is positioned at a predetermined position with respect to the papillary portion of the duodenum; therefore, the full auto mode is desirable in view of convenience. According to the present embodiment, it is possible to set an appropriate motion mode for these procedure steps.

The setting to the full auto mode in the positioning step in ERCP and the setting to the semi auto mode in the treatment step including the cannulation step are described in “. Switching of Motion Mode”, etc.

Further, in the present embodiment, the control device 600 may include, as a motion mode, a manual mode in which the intervention by automatic control is not performed with respect to the manual control. The control device 600 may switch the motion mode between the full auto mode, the semi auto mode, and the manual mode, according to the step of endoscopic retrograde cholangiopancreatography. The medical instrument may include the endoscope 100 in which the endoscopic operation, which is the instrument motion, is electrically driven. In the step of inserting the endoscope 100 into the papillary portion of the duodenum, the control device 600 may set the manual mode as the motion mode.

In ERCP, depending on the contents of the procedure, the operator may desire to perform all operations by himself/herself. For example, when the endoscope 100 is inserted into the papillary portion of the duodenum, the operator presumably more quickly inserts the endoscope by manual operation, and assistance is not really necessary. According to the present embodiment, by setting the manual mode in the endoscope insertion step, the operator can perform all operations by himself/herself in the endoscope insertion step without the intervention by the automatic control.

The endoscope insertion step is described in “Explanation of ERCP”, FIG. 5 in “Procedure Flow and Medical System According to The Present Embodiment”, etc. The setting to the manual mode in the endoscope insertion step is described in “. Switching of Motion Mode”, etc.

Further, in the medical system 10, the electrical driving of the bending movement of the endoscope 100 is not limited to the structure of the present embodiment. For example, it may be structured such that an attachment equipped with an electric motor is detachably attached to a bending operation knob of a non-electrically-driven endoscope. The drive control device 200 and the attachment are structured to communicate with each other, and, upon reception of a bending control signal from the drive control device 200, the attachment is driven to perform the bending. In this case, the manual control and the automatic control can be switched by attaching and detaching the attachment. It may also be arranged such that a handle capable of controlling the driving of the drive control device 200 is detachably attached to a motor unit for bending control corresponding to the drive control device 200. In this case, the manual control and the automatic control can be switched by attaching and detaching the handle.

The present embodiment may be implemented as a cannulation method as follows. More specifically, the cannulation method uses the endoscope 100. In the endoscope 100, the endoscopic operation is electrically driven, thereby capturing an endoscope image. The endoscopic operation is at least one of forward and backward movement of the insertion section 110, the bending angle of the bending section 102 of the insertion section 110, and the rolling rotation of the insertion section 110. The cannulation method includes a step of setting the full auto mode that performs automatic control of the electrically-driven endoscopic operation in the positioning step of positioning the endoscope 100 at the papillary portion of the duodenum. The cannulation method includes a step of setting the semi auto mode that performs intervention by automatic control with respect to the manual control of controlling the electrically-driven endoscopic operation based on an operation input, in the cannulation step of inserting the treatment tool 400 into a biliary duct.

In accordance with one of some aspect, there is provided a medical system comprising:

an endoscope with a tip portion from which a treatment tool is projectable; and

a control device,

wherein, after the tip portion of the endoscope is positioned with respect to an opening of a lumen and the treatment tool is inserted into the lumen, the control device operates in a treatment mode to maintain a position of the treatment tool in the lumen.

In accordance with one of some aspect, there is provided an operation method for a medical system, the method comprising performing an operation in a treatment mode, in which after a tip portion of an endoscope is positioned with respect to an opening of a lumen and a treatment tool is inserted into the lumen, the endoscope with the tip portion from which the treatment tool is projectable, a position of the treatment tool in the lumen is maintained.

In accordance with one of some aspect, there is provided a non-transitory information storing medium that stores a program that causes a computer to execute a method, the method comprising performing an operation in a treatment mode, in which after a tip portion of an endoscope is positioned with respect to an opening of a lumen and a treatment tool is inserted into the lumen, the endoscope with the tip portion from which the treatment tool is projectable, a position of the treatment tool in the lumen is maintained.

The present embodiment relates to automatic control performed when treatment in accordance with endoscopic retrograde cholangiopancreatography (ERCP) and a content of medical treatment is performed with use of an electrically driven medical system. The ERCP is an abbreviation for endoscopic retrograde cholangiopancreatography.

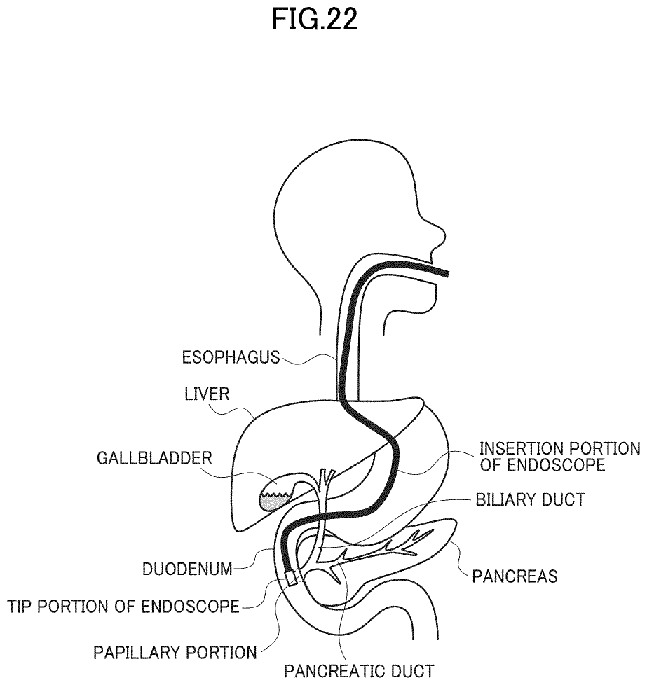

First, a content of manipulation of the ERCP is described. FIG. 22 illustrates organs and tissues that are related to the manipulation of the ERCP. Note that an organ has a unique structure in which a plurality of types of tissues gathers together, and has a specific function. In FIG. 22, the liver, the gallbladder, the pancreas, the esophagus, the stomach, and the duodenum correspond to the organs. The tissues are formed by related cells being coupled to each other, such as blood vessels, muscles, and skin. In FIG. 22, the biliary duct and the pancreatic duct correspond to the tissues.

The object of the treatment by the ERCP is the biliary duct. The biliary duct is a duct line for flowing biliary created by the liver to the duodenum. To approach the biliary duct with an endoscope, a treatment tool that is inserted through a channel of the endoscope is inserted from the papillary portion of the duodenum to the biliary duct while the endoscope remains to be maintained at a position of the duodenum. The papillary portion of the duodenum is hereinafter simply referred to as the papillary portion. The papillary portion is a region including an opening in which luminal tissues open to the duodenum, and not only the opening but also a structure in the periphery of the opening is referred to as the papillary portion. The opening of the luminal tissues is a portion, in which a common duct in which the biliary duct and the pancreatic duct join together, opens to the duodenum.

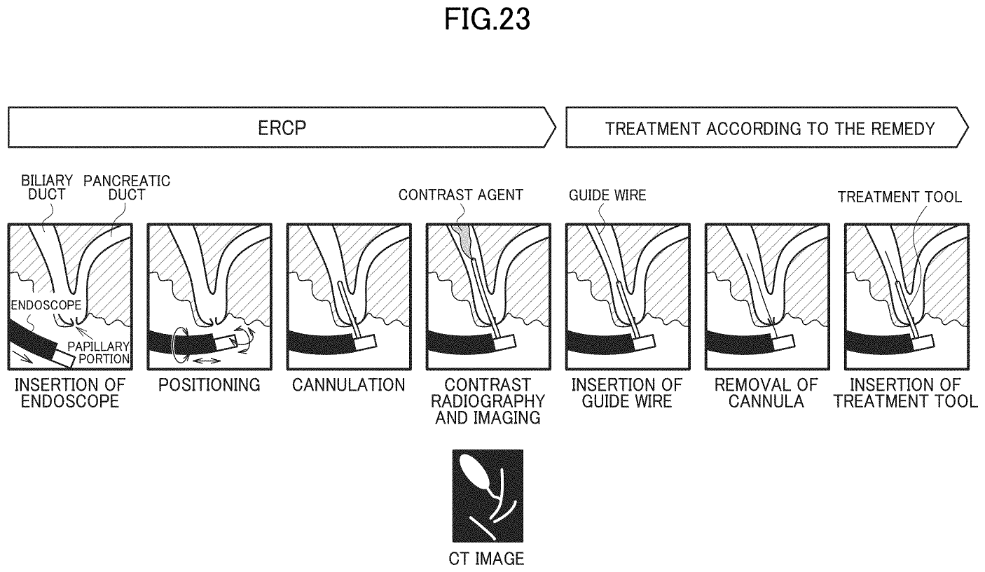

FIG. 23 shows a flow of the ERCP procedure. A side-view-type endoscope provided with a camera, an illumination lens, and an opening of a treatment tool channel on a side surface of the tip portion of the endoscope is used for the ERCP. Note that the camera is also referred to as an imaging device.

In an endoscope insertion step, an insertion portion of the endoscope is inserted from the mouth, by way of the esophagus and the stomach, into the duodenum. At this time, the insertion portion is inserted to a position where the papillary portion is roughly seen in a visual field of the endoscope. Subsequently, in a positioning step, the endoscope is aligned with the papillary portion. Specifically, the position of the tip portion of the endoscope is adjusted so that the papillary portion is within an imaging range of the camera of the endoscope. Alternatively, the position of the tip portion of the endoscope is adjusted so that the camera of the endoscope is at a correct position with respect to the papillary portion and is seen at the center of the field view.

Subsequently, in a cannulation step, a cannula is inserted from the papillary portion to the biliary duct. Specifically, the cannula is inserted into the treatment tool channel of the endoscope to project the cannula from a channel opening in the tip portion of the endoscope, a tip of the cannula is put in an opening of the common duct and inserted into the common duct, and furthermore, the cannula is inserted from a joint portion of the biliary duct and the pancreatic duct toward a direction of the biliary duct. Note that the cannulation is insertion of the cannula into the body. The cannula is a medical tube that is inserted into the body and used for a medical purpose.

Subsequently, in a contrast radiography and imaging step, a contrast agent is injected into the cannula and is poured from the tip of the cannula into the biliary duct. X-ray imaging or computed tomography (CT) imaging is performed in this state, whereby an X-ray image or a CT image, in which the biliary duct, the gallbladder, and the pancreatic duct are seen, is acquired. This is the manipulation of the ERCP, and thereafter various kinds of treatment are performed in accordance with results of diagnosis based on the X-ray image or the CT image. One example of the treatment will be described below.

In a guide wire insertion step, a guide wire is inserted into the cannula to project the guide wire from the tip of the cannula, and the guide wire is inserted into the biliary duct. In a cannula removal step, the cannula is removed while the guide wire is left inside the biliary duct. This leads to a state where only the guide wire projects from the tip of the endoscope and is left inside the biliary duct. Subsequently, in a treatment tool insertion step, the treatment tool is inserted into the biliary duct along the guide wire. The treatment tool is, for example, the basket treatment tool exemplified in the present embodiment, but may be a stent or the like. Note that the stent is a treatment tool that is inserted into a narrowed part of the biliary duct to expand the narrowed part, and is left after insertion to maintain an expanded state of the narrowed part.

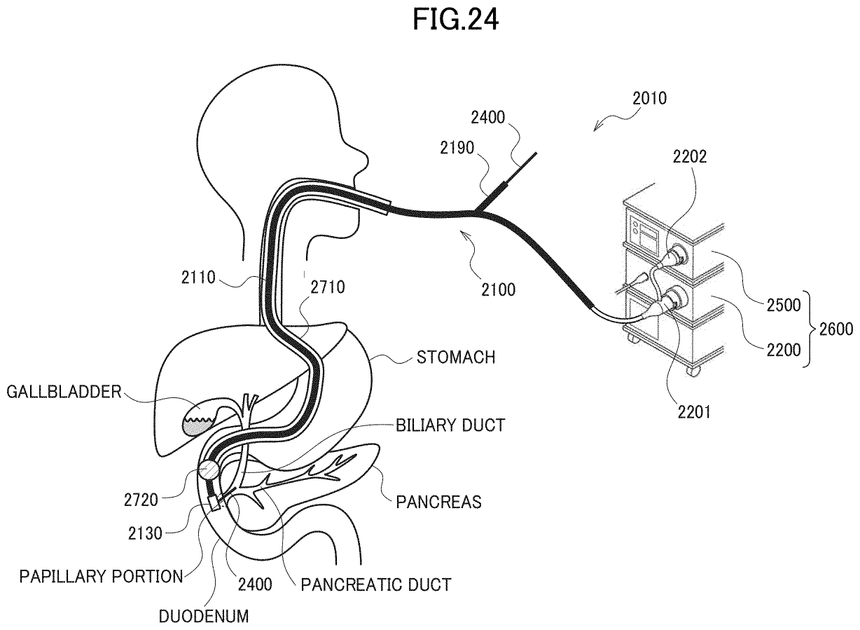

FIG. 24 illustrates a configuration example of a medical system 2010 in accordance with the present embodiment. The medical system 2010 includes an endoscope 2100, a treatment tool 2400, and a control device 2600. The control device 2600 includes a drive control device 2200 to which a connector 2201 is connected, and a video control device 2500 to which a connector 2202 is connected. The endoscope 2100 is detachably connected to the control device 2600 by the connectors 2201 and 2202.

Note that the medical system 2010 is also referred to as an endoscope system. Additionally, in a case where the endoscope 2100 is of an electrically driven type, the medical system 2010 can be also referred to as an electrically driven endoscope system. While FIG. 24 exemplifies the medical system 2010 using the endoscope 2100 of the electrically driven type, the endoscope 2100 may be of a manual operation type.

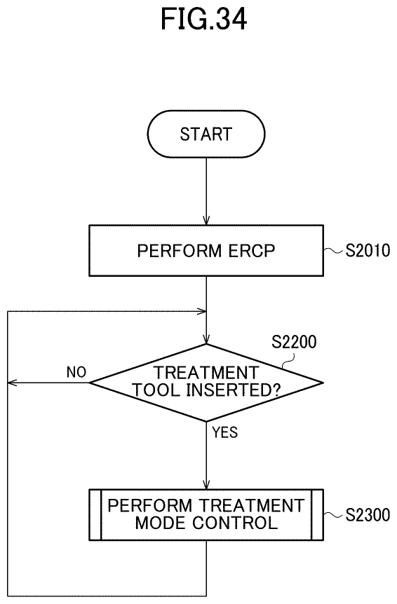

The control device 2600 controls each section of the drive control device 2200, the video control device 2500, and the like, and plays a main role in performing the processing flow that will be described later with reference to FIG. 34 or subsequent drawings. The control device 2600 includes the following hardware. The hardware can include at least one of a circuit that processes a digital signal or a circuit that processes an analog signal. For example, the hardware can include one or more circuit devices mounted on a circuit board, or one or more circuit elements. The one or more circuit devices are, for example, integrated circuits (ICs), field-programmable gate array (FPGA) circuits, or the like. The one or more circuit elements are, for example, resistors, capacitors, or the like.

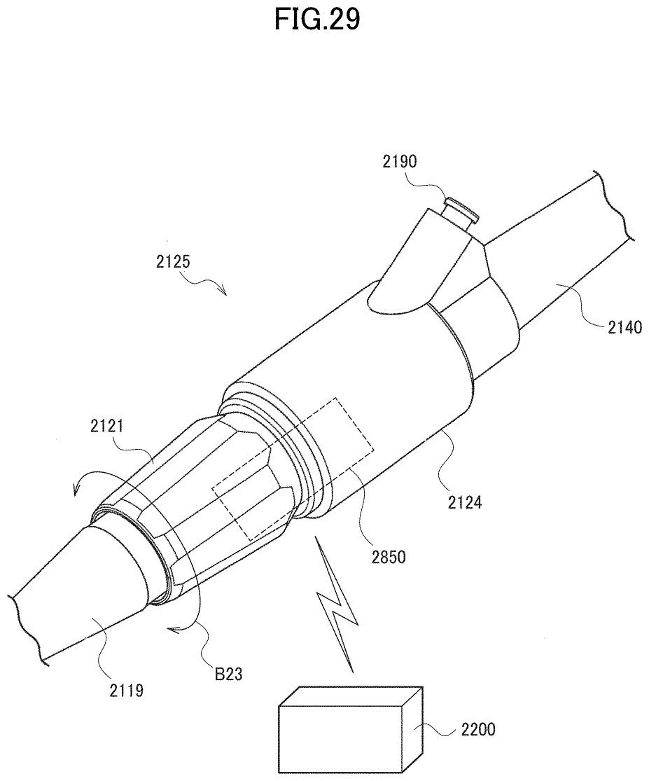

In addition, the control device 2600 is implemented by inclusion of at least one of the following processors. The control device 2600 includes a memory that stores information, and a processor that operates based on the information stored in the memory. The information is, for example, a program, various kinds of data, and the like. The processor includes hardware. Note that various kinds of processors such as a central processing unit (CPU), a graphics processing unit (GPU), and a digital signal processor (DSP) can be used. The memory may be a semiconductor memory such as a static random-access memory (SRAM) and a dynamic random-access memory (DRAM). The memory may be a register. The memory may be a magnetic storage device such as a hard disk drive (HDD). The memory may be an optical storage device such as an optical disk device. For example, the memory stores a computer-readable instruction. The instruction is executed by the processor, whereby part or all of functions of each section of the control device 2600 is implemented as processing. The instruction mentioned herein may be an instruction of an instruction set that is included in a program, or may be an instruction that instructs a hardware circuit included in the processor to operate. Furthermore, all or part of each section of the control device 2600 can be implemented by cloud computing, and each processing described later with reference to FIG. 29 or the like can be executed on the cloud computing.

The drive control device 2200 controls electric driving of the endoscope 2100 through the connector 2201. The drive control device 2200 can be implemented by including a processor similar to the above-mentioned processor. Although not illustrated in FIG. 24, an operation device for manually operating electric driving may be connected to the drive control device 2200.

The video control device 2500 receives an image signal from the camera arranged in a tip portion 2130 of the endoscope 2100 through the connector 2202, generates a display image from the image signal, and performs processing of displaying the display image on a display device, which is not illustrated. The video control device 2500 can be implemented by including a processor similar to the above-mentioned processor.

Note that the drive control device 2200 and the video control device 2500 are illustrated as individual devices in FIG. 24, but may be configured as an integrated device. In this case, the connectors 2201 and 2202 may be integrated as one connector. The following description will be given assuming that the control device 2600, the drive control device 2200, the video control device 2500 each include an individual processor, but the configuration is not limited thereto. For example, a processor or the like of the control device 2600 may fulfill a function as the drive control device 2200, and a processor or the like of the control device 2600 may fulfill a function as the drive control device 2200 and the video control device 2500.

The endoscope 2100 includes an insertion portion 2110. The insertion portion 2110 is a portion inserted into a lumen of a patient, and is configured to be flexible in a long and thin shape. Note that details of the endoscope 2100 including a configuration other than the insertion portion 2110 will be described later. An insertion opening 2190 of the treatment tool is arranged on the base end side of the insertion portion 2110, and the treatment tool channel for passing the treatment tool 2400 from the insertion opening 2190 to the opening of the tip portion 2130 is arranged inside the insertion portion 2110. The insertion opening 2190 of the treatment tool is also referred to as a forceps opening, but the treatment tool to be used is not limited to a forceps.

Note that a configuration example of the medical system 2010 in accordance with the present embodiment is limited to the above-mentioned examples, and the medical system 2010 may further include, for example, an overtube 2710 and a balloon 2720 as illustrated in FIG. 24.

The overtube 2710 is a tube that covers the insertion portion 2110 of the endoscope 2100 and that is variable in hardness. In a state where the endoscope 2100 and the overtube 2710 are inserted into the body, at least a curved portion of the insertion portion 2110 is in an exposed state from a tip of the overtube 2710. The curved portion is a portion configured to be bent at an angle in accordance with a bending operation in the vicinity of the tip of the insertion portion 2110. In addition, a base end of the overtube 2710 is outside the body, and the base end side of the insertion portion 2110 is exposed from the base end of the overtube 2710.

The balloon 2720 is arranged in the vicinity of the outside tip of the overtube 2710. For example, an operator performs an operation of inflating the balloon 2720 arranged in the vicinity of the tip of the overtube 2710 to fix the tip of the overtube 2710 to the duodenum with the balloon 2720. This can fix the position of the tip of the overtube 2710.

The operator then performs, for example, an operation of hardening the overtube 2710. With this operation, an insertion path of the insertion portion 2110 is fixed. As a result, the insertion path of the insertion portion 2110 can be maintained. Note that a method of hardening the overtube 2710 is publicly known and thus a description thereof is omitted.

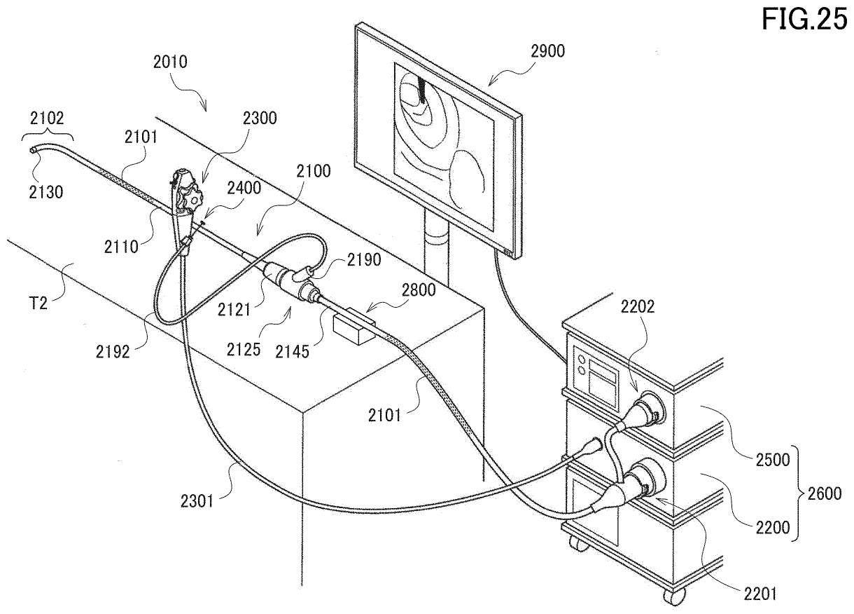

The medical system 2010 in accordance with the present embodiment may be implemented as a configuration example of observing or performing treatment on the inside of the body of the patient lying on an operating table T2, as illustrated in FIG. 25. The medical system 2010 in FIG. 25 includes the endoscope 2100, the control device 2600, an operation device 2300, the treatment tool 2400, an advancing/retreating driving device 2800, and a display device 2900.

The endoscope 2100 is a device that is inserted into the lumen of the patient for observation of a diseased part. In the present embodiment, an insertion side of the endoscope 2100 into the lumen of the patient is referred to as a “tip side”, and a mounting side of the endoscope 2100 to the control device 2600 is referred to as a “base end side”. In addition, movement of the endoscope 2100, the treatment tool 2400, or the like toward the tip side may be referred to as “advancing”, movement of the treatment tool 2400 or the like toward the base end side may be referred to as “retreating”, and advancing and retreating may be simply referred to as “advancing/retreating”.

The endoscope 2100 includes the insertion portion 2110 described above with reference to FIG. 24, a coupling element 2125, an extracorporeal flexible portion 2145, and the connectors 2201 and 2202. The insertion portion 2110, the coupling element 2125, the extracorporeal flexible portion 2145, and the connectors 2201 and 2202 are connected to one another in this order from the tip side.

The insertion portion 2110 includes a curved portion 2102, an extracorporeal flexible portion that connects a base end of the curved portion 2102 and the coupling element 2125 to each other, and the tip portion 2130 arranged at the tip of the curved portion 2102. An internal path 2101 is arranged inside the insertion portion 2110, the coupling element 2125, and the extracorporeal flexible portion 2145, and a curved wire that passes through the internal path 2101 is connected to the curved portion 2102. The drive control device 2200 drives the wire through the connector 2201 to perform a bending operation of the curved portion 2102. A wire for the raising base to be connected to the raising base arranged in the tip portion 2130 passes through the internal path 2101 and is connected to the connector 2201. The drive control device 2200 drives the wire for the raising base to change a rising angle of the treatment tool 2400 that projects from the side surface of the tip portion 2130. The camera, the illumination lens, and the opening of the treatment tool channel are arranged on the side surface of the tip portion 2130. An image signal line that connects the camera and the connector 2202 to each other is arranged on the internal path 2101, and an image signal is transmitted from the camera to the video control device 2500 through the image signal line. The video control device 2500 displays an endoscope image generated from the image signal on the display device 2900.

The insertion opening 2190 of the treatment tool and a roll operating portion 2121 are arranged in the coupling element 2125. The treatment tool channel is arranged on the internal path 2101, one end of the treatment tool channel opens in the tip portion 2130, and the other end thereof opens in the insertion opening 2190 of the treatment tool. An extension tube 2192 that extends from the insertion opening 2190 to the operation device 2300 is connected to the insertion opening 2190. The treatment tool 2400 is inserted from an opening of the extension tube 2192 on the operation device 2300 side, passes through the insertion opening 2190 and the treatment tool channel, and projects from the opening of the tip portion 2130. Note that the extension tube 2192 may be omitted and the treatment tool 2400 may be inserted from the insertion opening 2190. The roll operating portion 2121 is attached to the coupling element 2125 to be rotatable about an axis line direction of the insertion portion 2110. A rotating operation of the roll operating portion 2121 rolls the insertion portion 2110. Note that the roll operating portion 2121 may be driven by a manual operation, or may be capable of being electrically driven. While a description of a detailed mechanism is omitted because it is publicly known, a mechanism for rolling the tip portion 2130 independently of the insertion portion 2110 may be arranged separately.

The advancing/retreating driving device 2800 is a driving device that electrically drives the insertion portion 2110 to advance/retreat the insertion portion 2110. For example, the extracorporeal flexible portion 2145 is detachable from the advancing/retreating driving device 2800, and the advancing/retreating driving device 2800 slides the extracorporeal flexible portion 2145 in the axis line direction in a state where the extracorporeal flexible portion 2145 is mounted on the advancing/retreating driving device 2800, whereby the insertion portion 2110 advances/retreats. While FIG. 25 illustrates an example in which the extracorporeal flexible portion 2145 and the advancing/retreating driving device 2800 are detachable, the configuration is not limited thereto, and the coupling element 2125 and the advancing/retreating driving device 2800 may be configured to be detachable.

The operation device 2300 is detachably connected to the drive control device 2200 through an operation cable 2301. The operation device 2300 may perform wireless communication with the drive control device 2200 instead of wired communication. When the operator operates the operation device 2300, a signal of the operation input is transmitted to the drive control device 2200 through the operation cable 2301, and the drive control device 2200 electrically drives the endoscope 2100 so as to perform an endoscope operation in accordance with the operation input based on the signal of the operation input. The operation device 2300 includes five or more channels of operation input sections corresponding to advancing/retreating of the endoscope 2100, a bending operation in two directions, rolling, and an operation of a raising base 2134. Note that in a case where there is a non-electrically driven operation among these operations, an operation input section for the operation may be omitted. Each operation input section includes, for example, a dial, a joystick, an arrow key, a button, and a switch, a touch panel, and/or the like.

The drive control device 2200 drives a built-in motor based on an operation input to the operation device 2300 to electrically drive the endoscope 2100. Alternatively, in a case where the motor is an external motor outside the drive control device 2200, the drive control device 2200 transmits a control signal to the external motor based on the operation input to the operation device 2300 and controls electric driving. In addition, the drive control device 2200 may drive a built-in pump or the like based on the operation input to the operation device 2300 and cause the endoscope 2100 to perform air supply/suction. The air supply/suction is performed through an air supply/suction tube arranged on the internal path 2101. One end of the air supply/suction tube opens in the tip portion 2130 of the endoscope 2100, and the other end thereof is connected to the drive control device 2200 through the connector 2201. Note that the treatment tool channel may be extended to the connector 2201, and the treatment tool channel may also serve as the air supply/suction tube.

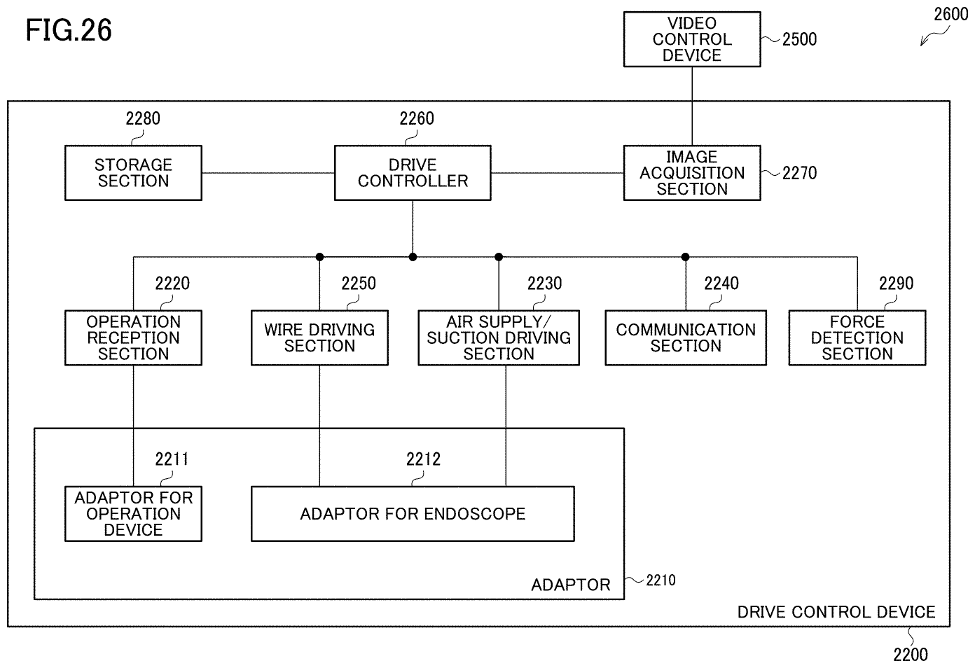

A block diagram in FIG. 26 illustrates a detailed configuration example of the drive control device 2200. The drive control device 2200 includes an image acquisition section 2270, a storage section 2280, a drive controller 2260, an operation reception section 2220, a wire driving section 2250, an air supply/suction driving section 2230, a communication section 2240, and an adaptor 2210.

The adaptor 2210 includes an adaptor for the operation device 2211 to which the operation cable 2301 is detachably connected and an adaptor for the endoscope 2212 to which the connector 2201 of the endoscope 2100 is detachably connected.

The wire driving section 2250 performs driving for the bending operation of the curved portion 2102 of the endoscope 2100 or the operation of the raising base of the treatment tool 2400, based on a control signal from the drive controller 2260. The wire driving section 2250 includes a motor unit for the bending operation that drives the curved portion 2102 of the endoscope 2100 and a motor unit for the raising base that drives the raising base. The adaptor for the endoscope 2212 has a coupling mechanism for the bending operation for coupling to the curved wire on the endoscope 2100 side. The motor unit for the bending operation drives the coupling mechanism, whereby driving force of the driving is transmitted to the curved wire on the endoscope 2100 side. The adaptor for the endoscope 2212 has a coupling mechanism for the raising base for coupling to the wire for the raising base on the endoscope 2100 side. The motor unit for the raising base drives the coupling mechanism, whereby driving force of the driving is transmitted to the wire for the raising base on the endoscope 2100 side.

The air supply/suction driving section 2230 performs driving for air supply/suction of the endoscope 2100 based on the control signal from the drive controller 2260. The air supply/suction driving section 2230 is connected to the air supply/suction tube of the endoscope 2100 through the adaptor for the endoscope 2212. The air supply/suction driving section 2230 is provided with a pump or the like, supplies the air to the air supply/suction tube, and sucks the air from an air supply/suction tube 2172.

The communication section 2240 performs communication with a driving device arranged outside the drive control device 2200. Communication may be either wireless communication or wired communication. The driving device arranged outside is the advancing/retreating driving device 2800 that performs advancing/retreating, a roll driving device that performs rolling, or the like, and details thereof will be described later. The driving devices arranged outside may be an overtube driving device that changes hardness of the overtube 2710, a balloon driving device that changes a diameter of the balloon 2720, or the like.

The drive controller 2260 controls the advancing/retreating of the endoscope 2100, the bending operation and the rolling, the rising angle of the treatment tool 2400 formed by the raising base, and the air supply/suction by the endoscope 2100. In a case where control of the hardness of the overtube 2710 or the diameter of the balloon 2720 is performed by means of electric driving, the drive controller 2260 may perform the control. The drive controller 2260 can be implemented by, for example, the above-mentioned processor. For example, the storage section 2280, which will be described later, stores a computer-readable program. The program is executed by the processor, whereby functions of the drive controller 2260 are implemented as processing.

The image acquisition section 2270 is a communication interface that receives image data of the endoscope image from the video control device 2500 through wired communication or wireless communication. The image acquisition section 2270 outputs the received image data of the endoscope image to the drive controller 2260. The endoscopic image may be captured in real time after the contrast radiography in FIG. 23.

In addition, the image acquisition section 2270 acquires image data of a transmissive image of the abdomen of the patient. The image data is used in treatment mode control (step S2300), which will be described later with reference to FIG. 34. The transmissive image is, for example, an ERCP image captured by an X-ray imaging device for surgery or a CT device, or a magnetic resonance cholangiopancreatography (MRCP) image captured by a magnetic resonance imaging (MRI) device. MRCP is an abbreviation for magnetic resonance cholangiopancreatography. The transmissive image is captured at least when the treatment mode control (step S2300) is performed, but may be captured in real time after the contrast radiography illustrated in FIG. 23.

The storage section 2280 stores information of a program and the like regarding drive control for the endoscope 2100. The storage section 2280 can be implemented by a storage device such as the semiconductor memory and the magnetic storage device that have been described above. Note that the storage section 2280 may store part or the whole of the program regarding the flow described in FIG. 29 or the subsequent drawings.

The drive control device 2200 in accordance with the present embodiment may further include a force detection section 2290 as illustrated in FIG. 26. The force detection section 2290 detects stress applied to the tip of the endoscope 2100 from an output signal from a force sensor 2180 included in the endoscope 2100. The force sensor 2180 will be described later. Similarly, the force detection section 2290 detects stress applied to the tip of the treatment tool 2400 from an output signal from a force sensor 2480 included in the treatment tool 2400. The force sensor 2480 will be described later. Note that the force detection section 2290 may include, for example, an amplification circuit that amplifies an output signal from the force sensor 180 or the force sensor 2480, and an analog/digital (A/D) converter that performs A/D conversion on the output signal from the amplification circuit and outputs stress detection data to the drive controller 2260. The drive controller 2260 is capable of making determination about contact based on the stress detection data, which will be described in detail later.

Although not illustrated, the drive control device 2200 in accordance with the present embodiment may be capable of acquiring distance information output from a distance-measurement sensor such as a Time-of-Flight sensor.

Additionally, the drive controller 2260 may be capable of performing control in a plurality of types of operation modes. Examples of the plurality of types of operation modes include a manual mode in which the operator manually operates electric driving of the endoscope 2100 or the like, and an automatic mode in which electric driving of the endoscope 2100 or the like is automatically controlled based on the endoscope image. For example, the positioning step described above with reference to FIG. 23 may be performed in the automatic mode. This enables automation of the positioning step. Note that in the automatic mode, at least one of advancing/retreating of the endoscope 2100, the bending operation, or the rolling is only required to be automated. That is, part of the rising angle of the treatment tool 2400 formed by the raising base 2134, the control of hardness of the overtube 2710, the control of the diameter of the balloon 2720, the air supply/suction of the endoscope 2100, the advancing/retreating of the endoscope 2100, the bending operation, or the rolling may be manually operated.

In the present embodiment, each of the operations described above and a treatment mode, which will be described later with reference to FIG. 34, can also be combined with each other as appropriate. For example, the operator may switch between the manual mode and the automatic mode at a freely-selected timing during a period of time under control in the treatment mode, which will be described later.

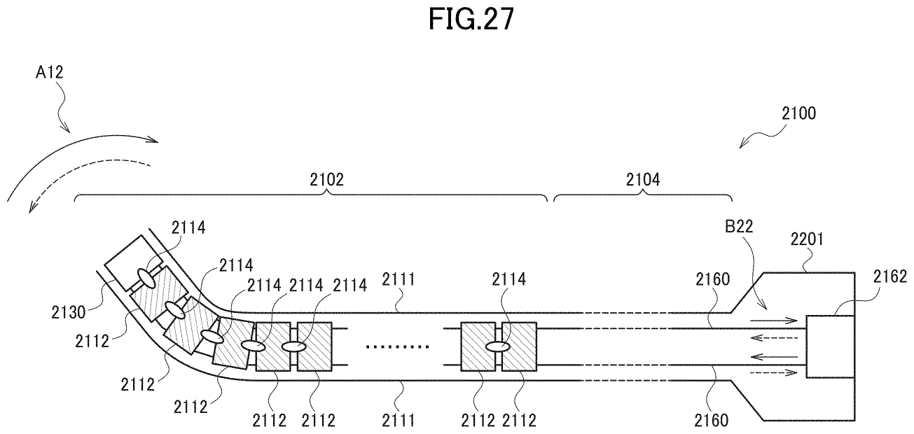

Subsequently, each constituent element driven by the drive control device 2200 is described in detail. FIG. 27 is a diagram schematically illustrating the endoscope 2100 including the curved portion 2102 and a driving mechanism for the curved portion 2102. The endoscope 2100 includes the curved portion 2102, a flexible portion 2104, and the connector 2201.

The curved portion 2102 and the flexible portion 2104 are covered with an outer sheath 2111. The curved portion 2102 includes a plurality of curve pieces 2112, and the tip portion 2130 that is coupled to a tip of the curve pieces 2112. The plurality of curve pieces 2112 and the tip portion 2130 are connected by corresponding coupling elements 2114 in series from the base end side to the tip portion, and have a multiple joint structure. The connector 2201 is provided with a coupling mechanism 2162 on the endoscope side. The coupling mechanism 2162 is connected to a coupling mechanism on the drive control device 2200 side. The connector 2201 being mounted on the drive control device 2200 enables electric driving of the bending operation. A curve wire 2160 is arranged inside the outer sheath 2111. One end of the curve wire 2160 is connected to the tip portion 2130. The curve wire 2160 penetrates through the plurality of curve pieces and passes through the flexible portion 2104, is folded back inside the coupling mechanism 2162, passes through the flexible portion 2104 again, and penetrates through the plurality of curve pieces 2112. The other end of the curve wire 2160 is connected to the tip portion 2130. Driving force from the wire driving section of the drive control device 2200 is transmitted as tractive force to the curve wire 2160 through the coupling mechanism 2162.

As indicated by an arrow of solid line in B22 in FIG. 27, when a wire on an upper side of the drawing is pulled, a wire on a lower side of the drawing is pushed, whereby a multiple joint of the curve pieces 2112 is bent in an upper direction in the drawing. With this operation, as indicated by an arrow of solid line in A12, the curved portion 2102 is curved in the upper direction in the drawing. In a case where the wire on the lower side of the drawing is pulled as indicated by an arrow by dotted lines in B22, the curved portion 2102 is similarly curved in a lower direction in the drawing as indicated by dotted line in A12. Note that the curved portion 2102 is capable of being curved independently in two directions that are mutually orthogonal to each other. FIG. 27 illustrates a curve mechanism only for one direction, but two pairs of curve wires are actually arranged. Each curve wire is pulled independently by the coupling mechanism 2162, and can thereby be curved independently in two directions.

Note that a mechanism for electrically driving bending is not limited thereto. For example, a motor unit may be arranged in substitution for the coupling mechanism 2162. Specifically, the driving for the bending operation may be performed by the drive control device 2200 transmitting a control signal to the motor unit through the connector 2201 and the motor unit pulling or loosening the curve wire 2160 based on the control signal.

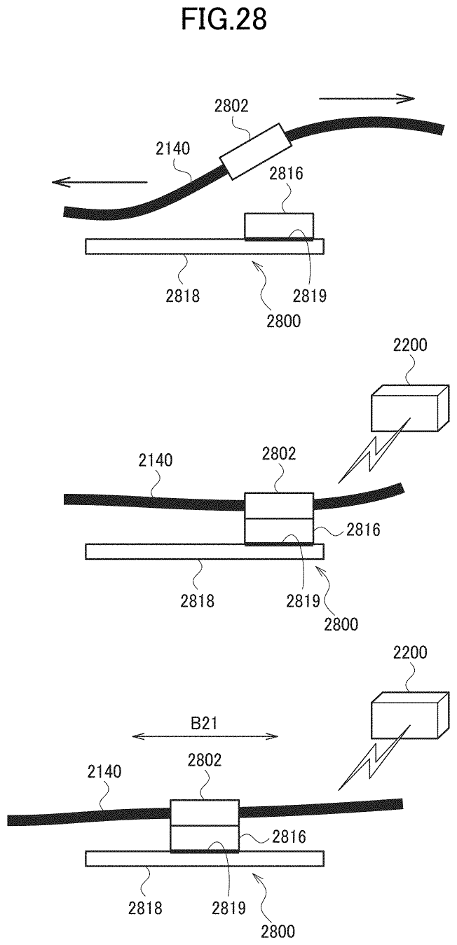

FIG. 28 illustrates a detailed configuration example of the advancing/retreating driving device 2800. The advancing/retreating driving device 2800 includes a motor unit 2816, a base 2818, and a slider 2819.

As illustrated in an upper drawing and a middle drawing, an extracorporeal flexible portion 2140 of the endoscope 2100 is provided with an attachment 2802 that is detachably mounted on the motor unit 2816. As illustrated in the middle drawing, mounting the attachment 2802 onto the motor unit 2816 enables electric driving for advancing/retreating. As illustrated in a lower drawing, the slider 2819 supports the motor unit 2816 so as to be capable of linearly moving with respect to the base 2818. The slider 2819 is fixed to an operating table. As indicated in B21, the drive control device 2200 transmits a control signal for advancing or retreating to the motor unit 2816 through wireless communication, and the motor unit 2816 and the attachment 2802 linearly move over the slider 2819 based on the control signal. Note that the drive control device 2200 and the motor unit 2816 may have a wired connection.

FIG. 29 is a perspective view illustrating the coupling element 2125 including a roll driving device 2850. The coupling element 2125 includes a coupling element main body 2124 and the roll driving device 2850.

An insertion opening 2190 of the treatment tool is arranged in the coupling element main body 2124, and is connected to the treatment tool channel inside the coupling element main body 2124. The coupling element main body 2124 has a cylindrical shape, and a cylindrical member that is coaxial with a cylinder of the coupling element main body 2124 is rotatably arranged inside the coupling element main body 2124. A base end portion of an intracorporeal flexible portion 2119 is fixed to the outside of the cylindrical member, and the base end portion serves as the roll operating portion 2121. This allows the intracorporeal flexible portion 2119 and the cylindrical member to be rotatable with respect to the coupling element main body 2124 about the axis line direction of the intracorporeal flexible portion 2119. The roll driving device 2850 is the motor unit arranged inside the coupling element main body 2124. As indicated in B23, the drive control device 2200 transmits a control signal for rolling to the roll driving device 2850 through wireless communication, and the roll driving device 2850 rotates the base end portion of the intracorporeal flexible portion 2119 with respect to the coupling element main body 2124 based on the control signal, whereby the intracorporeal flexible portion 2119 rolls. Note that the roll driving device 2850 may include a clutch mechanism, which switches between non-electric driving and electric driving of the rolling. Note that the drive control device 2200 and the roll driving device 2850 may have a wired connection using a signal line that passes the internal path 2101.

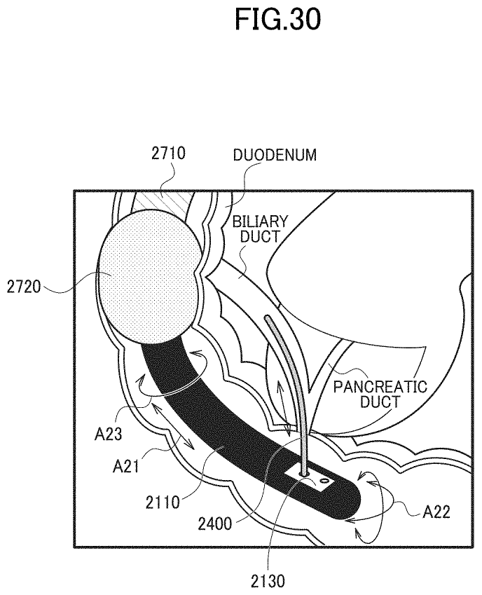

In this manner, with inclusion of various kinds of mechanisms exemplified in FIGS. 27 to 29, the medical system 2010 can implement advancing/retreating, bending, and rolling of the tip portion 2130 of the endoscope 2100. For example, as illustrated in FIG. 30, in the vicinity of the tip of the endoscope at the time of execution of the positioning step in FIG. 23, the medical system 2010 is capable of performing the advancing/retreating indicated in A21, the bending operation indicated in A22, or the rolling indicated in A23 on the tip portion 2130. Note that the advancing mentioned herein is movement toward the tip side along the axis line direction of the insertion portion 2110, and the retreating is movement toward the base end side along the axis line direction of the insertion portion 2110, as described above. The bending operation is an operation for bending the curved portion 2102 to change an angle of the tip portion 2130. The bending operation includes bending operations in two directions that are orthogonal to each other, and the bending operations can be independently controlled. One of the two directions that are orthogonal to each other is referred to as an upper/lower direction, and the other of the two directions is referred to as a left/right direction.

Note that it has been described above that the medical system 2010 in accordance with the present embodiment may further include the overtube 2710 and the balloon 2720, and the same applies to FIG. 30. In the medical system 2010, for example, fixing the balloon 2720 at a short distance away from the papillary portion toward the pylorus side of the stomach and combining the balloon 2720 and the overtube 2710 allows the balloon 2720 to freely move the curved portion 2102 and the tip portion 2130 that are exposed on the papillary portion side. This allows the drive control device 2200 to efficiently transmit electric drive from the base end side to the tip portion 2130 of the endoscope 2100.

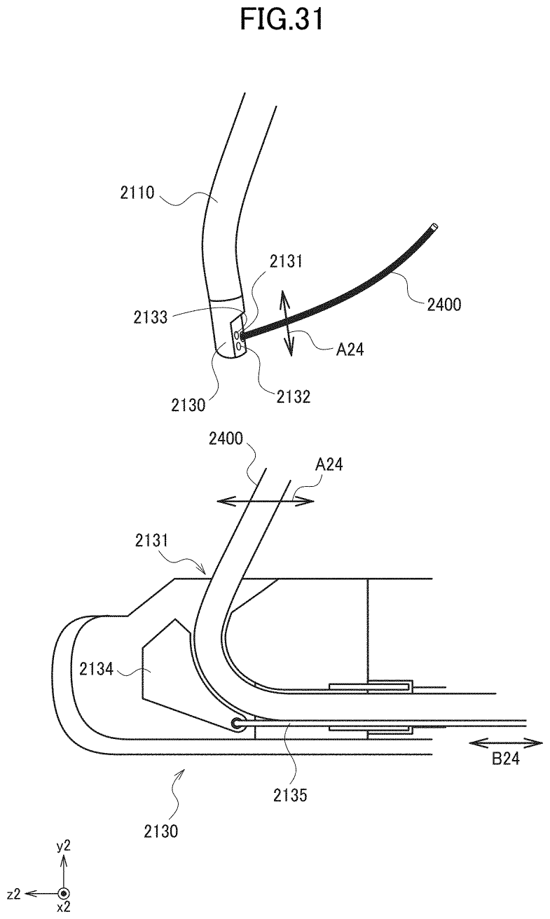

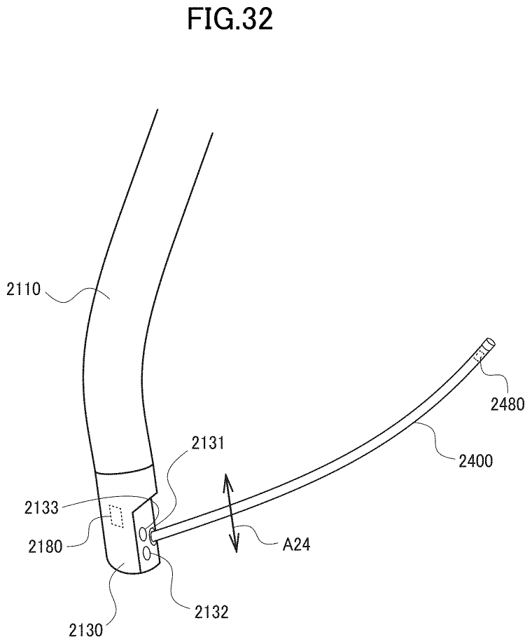

Subsequently, the tip portion 2130, the raising base 2134, and the treatment tool 2400 are described. FIG. 31 illustrates a detailed configuration example of the tip portion 2130 of the endoscope, the tip portion including the raising base 2134 of the treatment tool 2400. Note that specific examples of the treatment tool 2400 in accordance with the present embodiment include the above-mentioned cannula, a cholangioscope, a basket treatment tool 1400, which will be described later. However, the treatment tool 2400 is not specifically limited, and the treatment tool 2400 illustrated, for example, in FIGS. 30 and 31 does not limit a specific shape or the like of the actual treatment tool 2400. The same applies to FIG. 32 or subsequent drawings. The upper drawing is an external view of the tip portion 2130. An opening 2131 of the treatment tool channel, a camera 2132, and an illumination lens 2133 are arranged on the side surface of the tip portion 2130. As illustrated in the lower drawing, assume that a direction that is parallel to the axis line direction of the tip portion 2130 is a z2-direction, a direction that is parallel to a line-of-sight direction of the camera 2132 is a y2-direction, and a direction orthogonal to the z- and y2-directions is an x2-direction. The lower drawing is a cross-sectional view of the tip portion 2130 in a plane that is parallel to a y2-z2 plane of the treatment tool channel and that passes through the opening 2131 of the treatment tool channel.

The tip portion 2130 includes the raising base 2134 and a wire for the raising base 2135. The raising base 2134 can pivotally move about an axis that is parallel to the x2-direction. One end of the wire for the raising base 2135 is connected to the raising base 2134, and the other end thereof is connected to the drive control device 2200 through the connector 2201. The wire driving section 2250 of the drive control device 2200 presses/pulls the wire for the raising base 2135 as indicated in B24, whereby the raising base 2134 pivotally moves, and the rising angle of the treatment tool 2400 changes as indicated in A24. The rising angle is an angle of the treatment tool 2400 that projects from the opening 2131, and can be defined by, for example, an angle formed between the treatment tool 2400 that projects from the opening 2131 and the z2-direction.

In addition, the drive controller 2260 may control the endoscope operation or the rising angle of the treatment tool 2400 so that the tip of the treatment tool 2400 faces a direction of the opening of the luminal tissues based on the endoscope image. Alternatively, the drive controller 2260 may control the endoscope operation or the rising angle of the treatment tool 2400 so that the tip of the treatment tool 2400 faces a traveling direction of the biliary duct based on the endoscope image. For example, assuming that information of the traveling direction of the biliary duct is added to a criterion image, the drive controller 2260 may perform control so that the tip of the treatment tool 2400 faces the traveling direction of the biliary duct based on the information. Note that the traveling direction mentioned herein is a two-dimensional direction on the endoscope image. That is, on the endoscope image, the drive controller 2260 controls the endoscope operation or the rising angle of the treatment tool 2400 so that the traveling direction of the biliary duct and the direction the treatment tool 2400 faces become substantially parallel. However, in a case where three-dimensional information of the traveling direction of the biliary duct can be obtained from a CT image or the like, the drive controller 2260 may perform control so that the traveling direction of the biliary duct and the direction the treatment tool 2400 faces to be substantially parallel.

The tip portion 2130 in accordance with the present embodiment may further include the force sensor 2180. For example, as illustrated in FIG. 32, the tip portion 2130 is covered with a hard housing made of metal, resin, or the like, and the force sensor 2180 is fixed to the housing. Examples of the force sensor 2180 include a strain gauge. The strain gauge is a mechanical sensor that measures strain of the housing. The tip portion 2130 comes in contact with organs or the like, and the housing strains. The strain gauge detects the strain, and thereby detects stress of contact. The strain gauge includes a thin insulating material and a metal foil resistive element arranged on the insulating material. When stress is applied to an object to which the strain gauge is attached, the strain gauge strains together with the object, and a resistance value of the metal foil resistive element is changed by the strain. The drive control device 2200 measures the resistance value to detect stress.

Similarly, the treatment tool 2400 in accordance with the present embodiment may further include the force sensor 2480. For example, as illustrated in FIG. 32, the force sensor 2480 is fixed to the tip portion of the treatment tool 2400. While FIG. 32 illustrates an example in which the medical system 2010 includes both the force sensor 2180 and the force sensor 2480, the configuration is not limited thereto, and the medical system 2010 may include either the force sensor 2180 or the force sensor 2480. While FIG. 32 illustrates one force sensor 2180 and one force sensor 2480, a plurality of force sensors 2180 may be arranged in the endoscope 2100, or a plurality of force sensors 2480 may be arranged in the treatment tool 2400.

Although not illustrated, for example, a signal line that connects the force sensor 2180 and the connector 2201 is arranged on the internal path 2101, and a force detection signal is transmitted from the force sensor 2180 to the drive control device 2200 through the signal line. Similarly, a signal line that connects the force sensor 2480 and the connector 2201 is arranged on the internal path 2101, and a force detection signal is transmitted from the force sensor 2480 to the drive control device 2200 through the signal line.

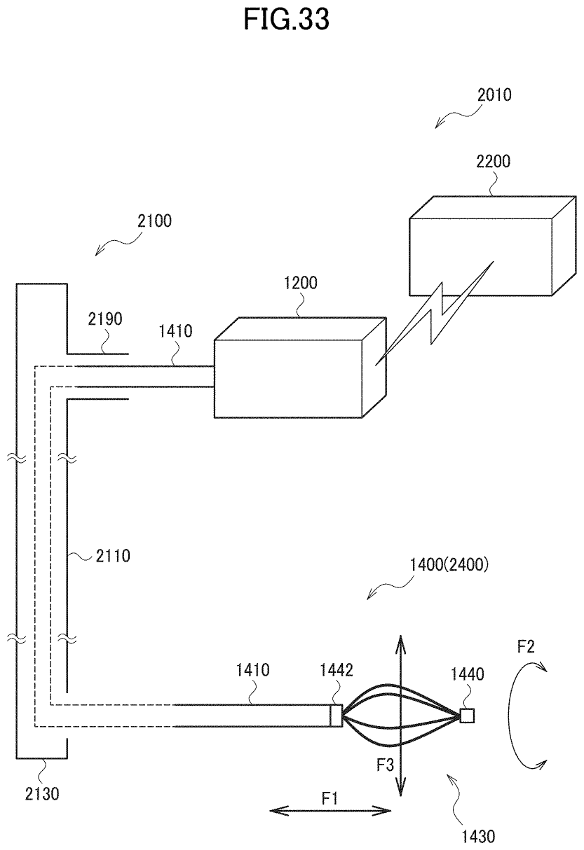

FIG. 33 exemplifies drive control of the basket treatment tool 1400 as an example of the treatment tool 2400 in accordance with the present embodiment. Note that in the endoscope 2100 in FIG. 33, illustration of a configuration other than the insertion opening 2190, the insertion portion 2110, and the tip portion 2130 is omitted. The endoscope 2100 in FIG. 33 is illustrated for conceptually indicating the insertion opening 2190, the insertion portion 2110, and the tip portion 2130, and does not limit a structure or the like of the endoscope 2100 in any way. For example, FIG. 33 illustrates that the rising angle of the treatment tool 2400 serving as the basket treatment tool 1400 is 90 degrees, but this does not limit the rising angle of the treatment tool 2400 in accordance with the present embodiment to 90 degrees.

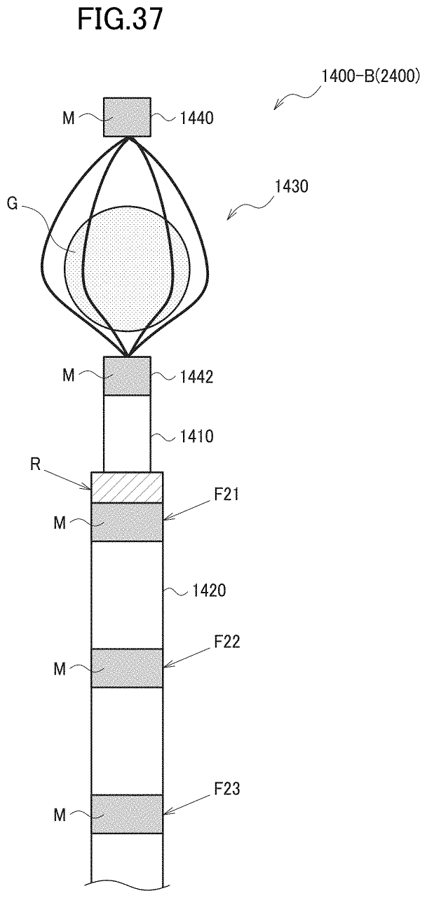

The basket treatment tool 1400 includes, for example, a first sheath 1410 and a basket 1430, and is electrically driven by a basket driving device 1200. For example, a basket treatment tool 1400-A for a purpose of extraction of a stone and a basket treatment tool 1400-B for a purpose of crushing of a stone are designed to be used differently. Additionally, the endoscope 2100 in accordance with the present embodiment includes at least one of the basket treatment tool 1400-A or the basket treatment tool 1400-B, but may include both the basket treatment tool 1400-A and the basket treatment tool 1400-B and project either of the basket treatment tools 1400 from the tip portion 2130 depending on the situation. Although not illustrated in FIG. 33, the basket treatment tool 1400-B for the purpose of crushing of the stone may further include a second sheath 1420, which will be described later in detail with reference to FIG. 37 and the like.

The basket treatment tool 1400, which will be described below, can be controlled in the above-mentioned automatic mode, but may be operable by the operator in the manual mode in a predetermined case. The predetermined case is a case where an unexpected malfunction occurs in the basket driving device 1200 or other cases, but may be, for example, a case where the operator wants to switch from the automatic mode to the manual mode or other cases, which will be described later in detail.

The basket driving device 1200 includes, for example, a mechanism such as a motor and a rack-and-pinion. This enables configuration of a slide mechanism for advancing/retreating an operation wire, which is not illustrated in FIG. 33, using rotative force of the motor. Accordingly, the basket driving device 1200 can control the advancing/retreating of the basket 1430. The basket driving device 1200 may use a similar mechanism to perform control for advancing/retreating the first sheath 1410 in a direction indicated in F1. Note that a detailed structure of a motor or the like included in the basket driving device 1200 is publicly known, and thus illustration thereof is omitted.

The basket driving device 1200 is connected to the drive control device 2200 through the communication section 2240 illustrated in FIG. 26. The connection mentioned herein is a wireless communication connection illustrated in FIG. 33, but may be a wired communication connection.

The drive control device 2200 controls an operation of the basket driving device 1200 through the communication connection. For example, the drive control device 2200 automatically controls crushing of a gallstone G using the basket treatment tool 1400, pulling of the basket treatment tool 1400, or the like. That is, the drive control device 2200 may control the basket treatment tool 1400 with the above-mentioned automatic mode. In the automatic mode, part of a drive mechanism of the basket treatment tool 1400 is only required to be automated.

As the above-mentioned manual mode, the operator may be able to manually operate the basket treatment tool 1400. That is, the drive control device 2200 may further include an operation input section for the basket treatment tool with which the operator can operate the basket treatment tool 1400. The operation input section for the basket treatment tool may be integrated with the operation device 2300 in FIG. 25, or may be configured as another controller aside from the operation device 2300.

The operation input section for the basket treatment tool may be arranged in the basket driving device 1200. The operation input section for the basket treatment tool includes, for example, a dial, a joystick, an arrow key, a button, and a switch, a touch panel, and/or the like. This allows, for example, the operator to advance/retreat the operation wire at a predetermined distance. Note that an outer appearance of the operation input section for the basket treatment tool is publicly known, so that specific illustration thereof is omitted.

In addition, part of functions of the basket driving device 1200 may be integrated with those of the drive control device 2200. For example, the wire driving section 2250 illustrated in FIG. 26 and the like may be capable of drive-controlling the operation wire of the basket treatment tool 1400.

The basket 1430 includes a plurality of elastic wires. The elastic wires on the tip side are tied together with a tip member 1440, and the elastic wires on the base end side are tied together with a coupling member 1442. Note that the number of elastic wires is not limited to four, and is determined as appropriate. For example, the number of elastic wires of the basket treatment tool 1400-A for the purpose of extraction of the stone may be made larger than the number of elastic wires of the basket treatment tool 1400-B for the purpose of crushing of the stone.

The elastic wires of the basket 1430 are self-urged so as to be curved outward in substantially identical shapes. In addition, the elastic wires are arranged at equal angular intervals when viewed from a direction from the tip member 1440 toward the first sheath 1410. With this configuration, the basket 1430 is formed in a contracted state while being pulled into the first sheath 1410, and is formed in a substantially basket shape in a state of projecting from the first sheath 1410.

The coupling member 1442 is arranged at the tip of the operation wire. For example, the operator inserts the first sheath 1410 into the biliary duct at a desired position through the tip portion 2130 in a state where the basket 1430 is pulled into the first sheath 1410. The operator then performs an operation of pushing the operation wire toward the tip side. With this operation, the basket 1430 is projected from the first sheath 1410, and the basket 1430 is formed in the substantially basket shape.

Note that the basket 1430 is illustrated in FIG. 33 in a substantially basket shape formed of curved lines, but the shape is not limited thereto. The basket 1430 may be, for example, in a substantially basket shape formed of folding points and straight lines. Various shapes have been proposed as publicly known shapes. Note that in the present embodiment, the shape of the basket 1430 is assumed to be optimized for extraction or the like of the gallstone G.

Note that the basket driving device 1200 may further include a mechanism of, after projection of the basket 1430 from the first sheath 1410, further contracting the substantially basket shape and expanding the basket 1430 again. For example, sliding the operation wire toward the base end side and partially fetching the coupling member 1442 and part of the basket 1430 in the first sheath 1410 enables contraction/expansion of the basket 1430 in a direction indicated in F3.

In addition, the basket driving device 1200 may be provided with, for example, a mechanism for rotating the coupling member 1442 and the operation wire about an axis of the guide wire. For example. the tip member 1440 is inserted through the guide wire, which is not illustrated in FIG. 33, and a handle or the like that rotates the operation wire about the guide wire is arranged, whereby the basket 1430 can be rotated in a direction indicated in F2. This configuration facilitates fetching of the gallstone G in the basket 1430. Note that a description or illustration of the guide wire is hereinafter omitted.

Additionally, the basket treatment tool 1400 in accordance with the present embodiment may couple part of the plurality of elastic wires to a rotary shaft of the coupling member 1442. The rotary shaft is not illustrated. With this configuration, for example, rotating the operation wire temporarily changes the shape of the basket 1430, and can facilitate fetching of the gallstone G.

In this manner, for example, the operator uses the basket driving device 1200 to perform advancing/retreating, opening/closing, rotation, or the like of the basket 1430 until the gallstone G is fetched in the basket 1430, while observing the transmissive image displayed on the display device 2900. After the gallstone G is fetched in the basket 1430, the operator uses the basket driving device 1200 to perform an operation of pulling out the basket treatment tool 1400 from the biliary duct, and can thereby remove the gallstone G from the papillary opening.

This enables, for example, drive control of the endoscope 2100 including the basket treatment tool 1400 serving as the treatment tool 2400. For example, the specification of United States Patent Application Publication No. 2007/0185377 discloses an example of advancing/retreating and opening/closing a basket forceps in accordance with an embedded program.

When the treatment tool 2400 is inserted into the lumen and treatment is performed, drive control for advancing/retreating is desirably performed while a posture of the treatment tool 2400 is maintained because the vicinity of the papillary opening is narrow. Especially in a case where the basket treatment tool 1400 as the treatment tool 2400 is used, it is likely that retreating the basket treatment tool 1400 in a state where a calculus or the like having a large diameter has been fetched in the basket 1430 applies a high load to the vicinity of the papillary opening unless the posture of the basket treatment tool 1400 is maintained. The method of maintaining the posture of the treatment tool 2400 inserted into the lumen has not been proposed so far.

A processing example in accordance with the present embodiment is described with reference to a flowchart in FIG. 34. Note that the flow in FIG. 34 is repeatedly performed on a periodic basis by timer interruption processing, but may be implemented as loop processing. While the above-description has been given assuming that the drive controller 2260 plays the main role in performing control or the like, but the following description will be given of a case where the control device 2600 plays the main role in performing control or processing as a typical example.

The control device 2600 first performs ERCP processing (step S2010). Specifically, the control device 2600 performs the flow regarding the above-mentioned ERCP that has been described with reference to FIG. 23. Thereafter, the control device 2600 performs processing of determining whether the treatment tool 2400 has been inserted (step S2200). When determining that the treatment tool 2400 has been inserted (YES in step S2200), the control device 2600 performs processing of operating the medical system 2010 under treatment mode control (step S2300). In contrast, when determining that the treatment tool 2400 has not been inserted (NO in step S2200), the control device 2600 performs step S2200 again. Note that a specific method in step S2200 will be described later.

A treatment mode control (step S2300) is a control for maintaining a position of the treatment tool 2400 in the lumen while performing the treatment. For example, in a case where the treatment tool 2400 is the basket treatment tool 1400, the control device 2600 executes step S2300 while the operator performs an operation of the basket treatment tool 1400.

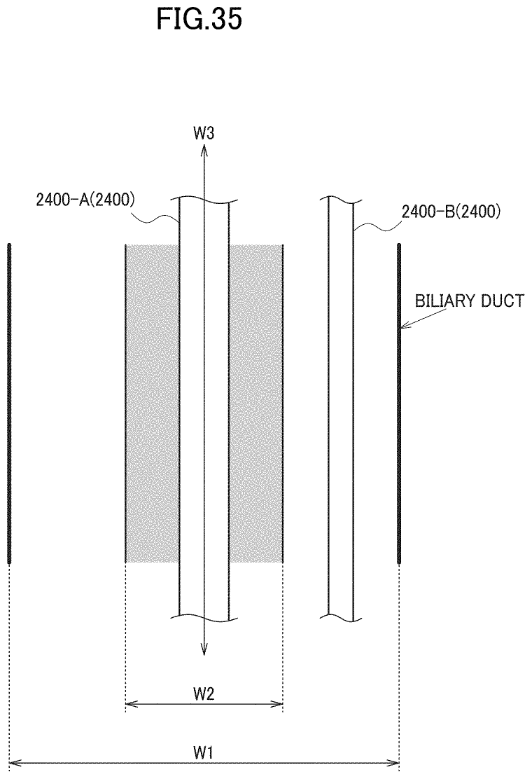

To maintain the position of the treatment tool 2400 in the lumen is, for example, to prevent the treatment tool 2400 positioned within a predetermined range in the lumen from being outside the predetermined range, but includes, for example, to return the treatment tool 2400 outside the predetermined range back to a position within the predetermined range. For example, as illustrated in FIG. 35, assume that there is the biliary duct having a width W1 as an example of the lumen. For example, when determining that treatment tool 2400-A is positioned within a range having a width W2 as the predetermined range, the control device 2600 determines that the treatment tool 2400-A is positioned in an appropriate range.

In contrast, as illustrated in FIG. 35, in a case where the treatment tool 2400-B is outside the range having the width W2, the control device 2600 determines that the treatment tool 2400-B is not positioned in the appropriate range. Note that FIG. 35 is a diagram conveniently illustrated for comparison, and does not indicate that the treatment tool 2400-A and the treatment tool 2400-B are simultaneously used in treatment.

Determination as to whether the treatment tool 2400 is positioned in the predetermined range in the lumen can be implemented by various kinds of methods. For example, the determination can be implemented by visual observation using the above-mentioned transmissive image or other methods, but details thereof will be described later. Alternatively, for example, the control device 2600 can implement the determination by performing processing of measuring resistance while periodically advancing/retreating the treatment tool 2400 in the treatment mode control (step S2300). The resistance is measured by, for example, the above-mentioned force sensor 2480. For example, the control device 2600 measures resistance in real time when the operator performs treatment in the manual mode, and performs processing of issuing a predetermined alarm in a case where the measured resistance exceeds a predetermined value. A value of the measured resistance being greater than the predetermined value means that the treatment tool 2400 receives contact resistance from an inner wall of the lumen, and can be equated with the treatment tool 2400 being positioned outside the predetermined range.

This allows the operator to recognize that the treatment tool 2400 is outside the predetermined range in the lumen. This allows the operator to perform a predetermined measures of coping to return the treatment tool 2400 outside the range having the width W2 in the lumen back to within the range having the width W2 in the lumen. Examples of the predetermined measures of coping include an operation of the treatment tool 2400 in an opposite direction of a direction in which the operator performs an operation on the treatment tool 2400 until the predetermined alarm is issued. Until the predetermined alarm stops, the operator continues to perform this predetermined measures of coping. This allows the treatment tool 2400 to be returned to a position within the predetermined range in lumen.

In this manner, the medical system 2010 includes the endoscope 2100 with the tip portion 2130 from which the treatment tool 2400 is projectable, and the control device 2600. After the tip portion 2130 of the endoscope 2100 is positioned with respect to an opening of the lumen and the treatment tool is inserted into the lumen, the control device 2600 operates in a treatment mode to maintain a position of the treatment tool 2400 in the lumen.

The medical system 2010 in accordance with the present embodiment includes the endoscope 2100 and the control device 2600, and thereby enables structuring of the system of controlling the endoscope 2100. After the tip portion 2130 of the endoscope 2100 is positioned with respect to the opening of the lumen and the treatment tool 2400 is inserted into the lumen, the control device 2600 operates in the treatment mode to maintain the position of the treatment tool 2400 in the lumen. Thus, when a situation where the posture of the treatment tool 2400 in the lumen is not appropriate occurs, the control device 2600 can bring the posture of the treatment tool 2400 into an appropriate state. This enables structuring of the medical system 2010 that appropriately handles the treatment tool 2400 inserted into the lumen. This allows the operator or the like to safely and appropriately perform the treatment on the lumen using the endoscope 2100 and the treatment tool 2400.

The method in accordance with the present embodiment may be implemented as an operation method for the medical system 2010. That is, after the tip portion 2130 of the endoscope 2100, with the tip portion 2130 from which the treatment tool 2400 is projectable, is positioned with respect to the opening of the lumen and the treatment tool 2400 is inserted into the lumen, the operation method for the medical system 2010 in accordance with the present embodiment includes a step of operating in the treatment mode to maintain the position of the treatment tool 2400 in the lumen. This enables obtaining of an advantageous effect that is similar to the above-mentioned advantageous effect.

The method in accordance with the present embodiment may be implemented as a non-transitory information storage medium. That is, the non-transitory information storage medium in accordance with the present embodiment stores a program that causes a computer to execute, after the tip portion 2130 of the endoscope 2100, with the tip portion 2130 from which the treatment tool 2400 is projectable, is positioned with respect to the opening of the lumen and the treatment tool 2400 is inserted into the lumen, a step of operating in the treatment mode to maintain the position of the treatment tool 2400 in the lumen. This enables obtaining of an advantageous effect that is similar to the above-mentioned advantageous effect.

The method in accordance with the present embodiment is not limited to the above-mentioned method, and can be modified in various manners. While the above-description has been given of the example in which the control device 2600 measures resistance while the operator operates the treatment tool 2400 in the manual mode, an example of electrically controlling the treatment tool 2400 in the automatic mode may be implemented. That is, the control device 2600 performs, for example, electric control for displacing the treatment tool 2400-A in a direction indicated in W3 while maintaining the position of the treatment tool 2400-A that has been described with reference to FIG. 35 within the range of the width W2. In this case, in the treatment mode control (step S2300), when the measured resistance exceeds the predetermined value, the control device 2600 may perform processing of aborting automatic control of the treatment tool 2400 or the like. When aborting the automatic control of the treatment tool 2400 or the like, the control device 2600 may automatically switch from the automatic mode to the manual mode. In this manner, in the medical system 2010 in accordance with the present embodiment, the control device 2600, in the treatment mode, electrically controls the treatment tool 2400 to maintain the position of the treatment tool 2400 in the lumen. This enables structuring of the medical system 2010 that electrically controls the treatment tool 2400 while maintaining the posture of the treatment tool 2400.

For example, in the treatment mode control (step S2300), whether the treatment tool 2400 is positioned within the predetermined range in the lumen may be visually determined. Specifically, for example, whether the treatment tool 2400 is positioned within the predetermined range in the lumen may be determined using the above-mentioned transmissive image. For example, the operator observes the transmissive image displayed on the display device 2900 while the control device 2600 operates the treatment tool 2400 in the automatic mode. In this case, for example, when the operator determines that the treatment tool 2400 is not positioned in the predetermined range in the lumen based on the transmissive image, the operator stops the automatic mode, switches the automatic mode to the manual mode, and operates the treatment tool 2400 to be positioned within the predetermined range in the lumen.

For example, when the operator operates the treatment tool 2400 in the manual mode, the control device 2600 may perform processing of determining whether the treatment tool 2400 is positioned within the predetermined range in the lumen based on the captured transmissive image. For example, the control device 2600 performs processing of extracting an image of a luminal portion and an image of a treatment tool portion from the transmissive image captured in real time, and processing of comparing the extracted image of the luminal portion and the extracted image of the treatment tool portion to determine whether the treatment tool 2400 is positioned within the predetermined range.

The processing of determining whether the treatment tool 2400 is positioned within the predetermined range in the lumen based on the extracted images can be implemented, for example, by the following method. The control device 2600 performs processing of calculating coordinates of the center of gravity of the treatment tool portion from coordinates of each pixel in the image of the treatment tool portion, and processing of calculating coordinates of the center of gravity of the luminal portion from coordinates of each pixel of the image of the luminal portion. The control device 2600 then performs processing of determining whether a difference between the coordinates of the center of gravity of the treatment tool portion and the coordinates of the center of gravity of the luminal portion is within a predetermined range. In a case where the difference between the coordinates of the center of gravity of the treatment tool portion and the coordinates of the center of gravity of the luminal portion is within the predetermined difference, the control device 2600 determines that the treatment tool 2400 is positioned within the predetermined range in the lumen. The predetermined difference is determined based on, for example, a relationship between a value of the width W1 and a value of the width W2 in FIG. 35, but is only required to be determined by the operator or the like in accordance with the shape of the lumen or the like.

When determining that the treatment tool 2400 is not positioned within the predetermined range in the lumen, the control device 2600 performs processing of issuing a predetermined alarm. The operator then performs an appropriate measures of coping so that the treatment tool 2400 is positioned within the predetermined range in the lumen. In this manner, in the medical system 2010 in accordance with the present embodiment, the control device 2600, in the treatment mode, maintains the position of the treatment tool in the lumen based on the transmissive image including the image of the lumen. This enables structuring of the medical system 2010 that visually grasps that the appropriate posture of the treatment tool 2400 is maintained in the lumen.

For example, the control device 2600 may electrically control the treatment tool 2400 in the automatic mode, and perform processing of determining whether the treatment tool 2400 is positioned within the predetermined range based on the captured transmissive image, as described above. That is, in the treatment mode, the control device 2600 identifies the position of the treatment tool 2400 based on the transmissive image including the image of the lumen, and electrically controls the treatment tool 2400 to maintain the identified position of the treatment tool 2400 with respect to the lumen. This enables structuring of the medical system 2010 that electrically controls the treatment tool 2400 while visually grasping that the appropriate posture of the treatment tool 2400 is maintained in the lumen.

While the method of extracting the image of the treatment tool portion and the image of the luminal portion and calculating the coordinates of the center of gravity of each image has been exemplified in the above description, the method in accordance with the present embodiment is not limited thereto. The control device 2600, for example, may determine whether the treatment tool 2400 is positioned within the predetermined range in the lumen based on a shape of the extracted image of the treatment tool portion. For example, in a case where the treatment tool 2400 is the cholangioscope, which has a uniform diameter and is long and thin, the shape of the treatment tool 2400 can be approximated to a shape of a path through which the treatment tool 2400 is inserted into the lumen. The control device 2600 performs the processing of extracting the image of the treatment tool portion from the captured transmissive image, and can thereby identify an insertion path of the treatment tool 2400. The control device 2600 may then perform processing of determining whether the extracted shape of the treatment tool portion is a predetermined shape to determine whether the treatment tool 2400 is positioned within the predetermined range in the lumen.

The predetermined shape is, for example, a linear shape or a shape that is close to the linear shape, but may be set by the operator or the like as appropriate depending on the shape of the lumen. For example, the shape of the treatment tool 2400 when the insertion of the treatment tool 2400 is completed to a desired position may be the predetermined shape. This is because, if the treatment tool 2400 can be retreated while the shape at the time of completion of the insertion can be maintained, it is thought that there is an insignificant effect on the lumen and the like.

The control device 2600 performs, for example, processing of comparing the shape of the image of the treatment tool portion extracted in real time and a set predetermined shape. When determining that the shape of the image of the treatment tool portion can be approximated to the predetermined shape, the control device 2600 determines that the treatment tool 2400 is positioned within the predetermined range in the lumen, and continues electric control of the treatment tool 2400.

In contrast, when determining that the shape of the image of the treatment tool cannot be approximated to the predetermined shape, the control device 2600 determines that the treatment tool 2400 is positioned outside the predetermined range in the lumen. In this case, the control device 2600, for example, performs processing of aborting automatic control of the treatment tool 2400. In this manner, in the medical system 2010 in accordance with the present embodiment, the control device 2600 identifies the insertion path of the treatment tool 2400 based on the transmissive image, and electrically controls the endoscope 2100 so that the shape of the insertion path has the predetermined shape. This enables securing of the appropriate insertion path for the treatment tool 2400 and structuring of the medical system 2010 that maintains the posture of the treatment tool 2400.

While the above-description has been given of the example of applying the method in accordance with the present embodiment to the treatment tool 2400 having a simple shape such as the cholangioscope, the method in accordance with the present embodiment is not limited thereto, and can also be applied to the treatment tool 2400 having a more complicated shape. The following description will be given using the basket treatment tool 1400, which is a treatment tool for removing a calculus, as an example of the treatment tool 2400. As described above, the treatment using the basket treatment tool 1400 is treatment of fetching the gallstone G as a calculus in the biliary duct in the basket 1430, treatment of pulling out the basket treatment tool 1400 from the biliary duct in a state where the gallstone G has been fetched in the basket 1430, or the like. That is, in the medical system 2010 in accordance with the present embodiment, the treatment tool 2400 is the treatment tool for removing the calculus, and the control device 2600 performs control for removing the calculus using the treatment tool 2400 in the treatment mode. This enables structuring of the medical system 2010 that performs treatment for removing the calculus while maintaining the posture of the treatment tool 2400.

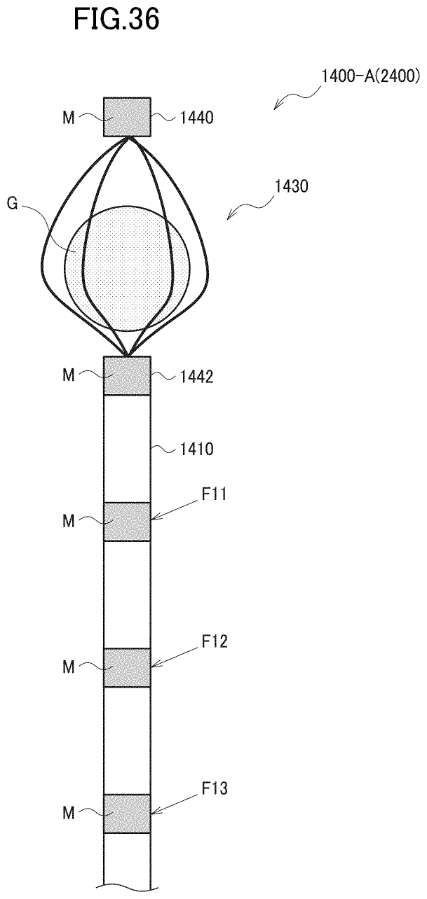

For example, assume that in the basket treatment tool 1400-A as an example of the treatment tool 2400, markers M each including a radiopaque material are added at respective positions indicated by F11, F12, and F13 in the first sheath 1410, as illustrated in FIG. 36. In this case, in the transmissive image displayed on the display device 2900, a portion including each marker M is displayed at a higher contrasting density than that of the vicinity of the portion. Note that the marker M may be added to a portion other than the first sheath 1410. For example, as illustrated in FIG. 36, the marker M may be added to the tip member 1440, the coupling member 1442, or the like. This allows the basket treatment tool 1400 having a complex shape as a whole to be conveniently handled as a simple shape such as the linear shape based on an array of markers M.

The control device 2600 then performs, for example, processing of pattern-matching a shape image of the marker M preliminarily stored in the storage section 2280 with the captured transmissive image, processing of calculating an approximation curve based on coordinates of each marker M subjected to pattern recognition, and processing of comparing the approximation curve based on a predetermined shape. When determining that the shape based on the approximation curve cannot be approximated to the predetermined shape, the control device 2600 then performs processing of issuing a predetermined alarm. Note that the control device 2600 may determine, in combination with the above-mentioned determination, whether a shape of a figure connecting the markers M displayed on the display device 2900 can be approximated to the predetermined shape.

In a case where the treatment tool 2400 is the basket treatment tool 1400-B further including the second sheath 1420 in addition to the first sheath 1410 as illustrated in FIG. 37, the markers M may be added to, for example, the second sheath 1420 at respective positions indicated in F21, F22, and F23.

In this manner, in the medical system 2010 in accordance with the present embodiment, the treatment tool 2400 includes the plurality of markers M, and the control device 2600 maintains the position of the treatment tool 2400 in the lumen based on the plurality of markers M seen in the transmissive image including the image of the lumen. This enables structuring of the medical system 2010 that more clearly grasps the posture of the treatment tool 2400 in the transmissive image. This can increase accuracy of control for maintaining the position of the treatment tool 2400.

Note that the method of observing the treatment tool 2400 to which the markers M are added may be applied to, for example, step S2200 in FIG. 34. For example, the control device 2600 performs processing of pattern-matching the shape image of the marker M preliminarily stored in the storage section 2280 with the captured transmissive image, and processing of determining whether the number of images of markers M subjected to the pattern-matching is a predetermined number or more. If the number of images of the markers M subjected to the pattern-matching is the predetermined number or more, the control device 2600 then determines YES in step S2200 because the treatment tool 2400 has been inserted enough into the lumen.

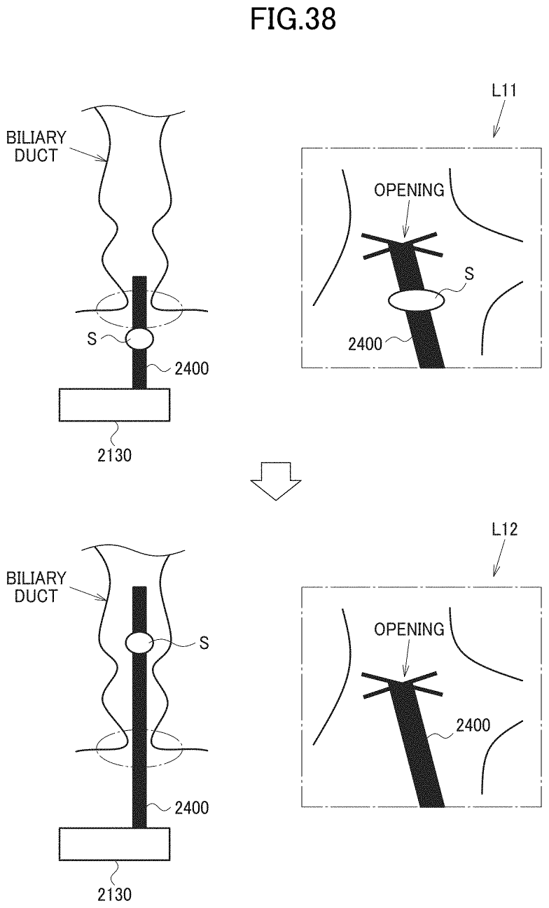

Alternatively, step S2200 can be implemented by a method of observing an endoscope image in substitution for the method of observing the transmissive image. For example, a marker S is added at a predetermined position on the tip portion side of the treatment tool 2400. The marker S mentioned herein is only required to be recognizable on the captured endoscope image, and the radiopaque material is not necessarily required. For example, the control device 2600 performs processing of pattern-matching an image of the marker S preliminarily stored in the storage section 2280 with the captured endoscope image. If the image of the marker S is present in the captured endoscope image as illustrated, for example, in L11 in FIG. 38, the control device 2600 determines NO in step S2200 because the treatment tool 2400 has not been inserted enough into the lumen. In contrast, the pattern image of the marker S is not present as illustrated, for example, in L12 in FIG. 38, the control device 2600 determines YES in step S2200 because the treatment tool 2400 has been inserted enough into the lumen. Note that FIG. 38 illustrates that the treatment tool 2400 includes one marker S, but may include a plurality of markers S, and the operator or the like can determine the number of markers S as appropriate. Note that a description or illustration of the marker S is hereinafter omitted.

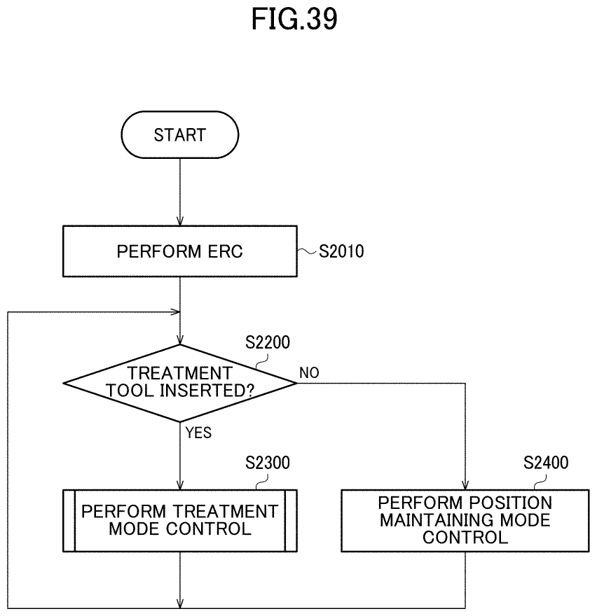

The processing in accordance with the present embodiment may be, for example, implemented as a processing example as indicated in a flowchart in FIG. 39. The processing example in FIG. 39 is different from the processing example in FIG. 34 in that, when determining that the treatment tool 2400 has not been inserted (NO in step S2200), the control device 2600 performs control for an operation under position maintaining mode control (step S2400).

A position maintaining mode in step S2400 is a mode that prioritizes maintaining of the position of the tip portion 2130 of the endoscope 2100 with respect to the opening of the lumen over maintaining of the position of the treatment tool 2400. In contrast, the treatment mode in step S2300 is a mode that prioritizes maintaining of the position of the treatment tool 2400 over maintaining of the position of the tip portion 2130 of the endoscope 2100.

In the position maintaining mode, for example, when the tip portion 2130 comes in contact with the intestinal wall of the duodenum, a force detection signal is transmitted from the force sensor 2180 to the control device 2600 through the force detection section 2290. The control device 2600 then electrically controls each driving mechanism of the endoscope 2100 so as to avoid the contact.

Similarly, for example, when the tip portion of the treatment tool 2400 comes in contact with the inner wall of the lumen, a force detection signal is transmitted from the force sensor 2480 to the control device 2600 through the force detection section 2290. The control device 2600 then electrically controls each driving mechanism of the endoscope 2100 and the treatment tool 2400 so as to avoid the contact.

Although illustration or the like is not given, the control device 2600 may perform, for example, processing of determining whether there is a possibility for contact with the tip portion 2130 or the tip of the treatment tool 2400. When determining that there is the possibility for the contact with the tip portion 2130 or the tip of the treatment tool 2400, the control device 2600 may electrically control each driving mechanism of the endoscope 2100 so as to avoid the contact. The processing of determining whether there is the possibility for the contact can be implemented by, for example, a method of arranging the above-mentioned distance-measurement sensor in the tip portion 2130 or the tip of the treatment tool 2400 and determining that there is the possibility for the contact when a distance measured by the distance-measurement sensor is less than or equal to a threshold, or other methods.

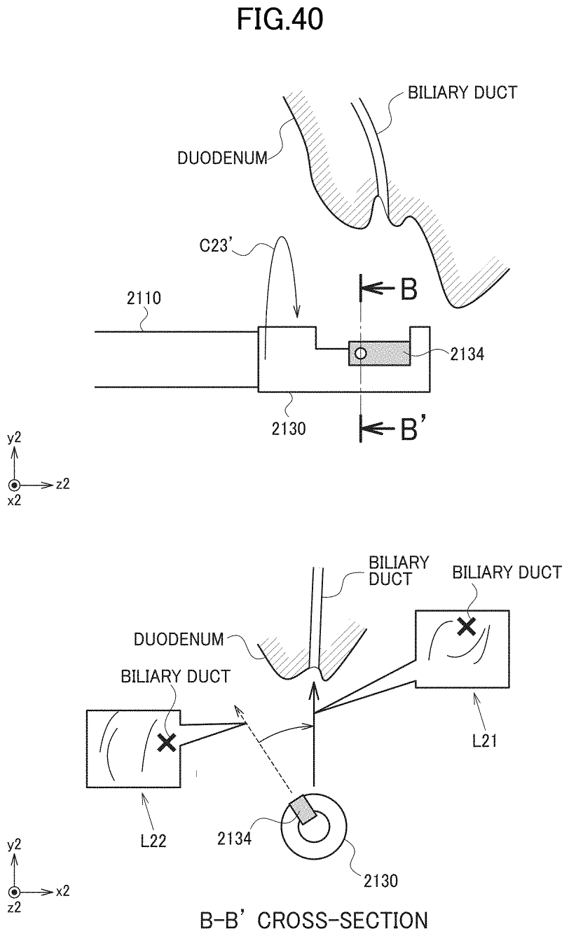

Additionally, for example, the control device 2600 may perform processing of temporarily storing an endoscope image captured by the camera 2132 at a position where the tip portion 2130 is desired to be maintained, and electrically driving, under the position maintaining mode control (step S2400), each driving mechanism of the endoscope 2100 so that an image captured by the camera 2132 in real time becomes identical to the temporarily stored endoscope image. FIG. 40 illustrates an example of the above-mentioned processing.

In FIG. 40, assume that the axis line direction of the tip portion 2130 is the z2-direction, and two directions orthogonal to the z2-direction are the x2- and y2-directions. For example, assume that the rolling of the base end portion of the insertion portion 2110 by the driving mechanism of the roll driving device 2850 described above with reference to FIG. 29 rolls the tip portion 2130 of the insertion portion 2110 about the z2-direction serving as a rotation axis as indicated in C23′ in the upper drawing of FIG. 40.

The lower drawing of FIG. 40 is a cross-sectional view along B-B′ line in the upper drawing of FIG. 40 when viewed from the z2-direction. For example, assume that the control device 2600 performs processing of aligning the position of the tip portion 2130 with the papillary portion using the ERCP (step S2010) in FIG. 34, and the endoscope image captured by the camera 2132 is displayed on the display device 2900, as indicated in, for example, L21.

Thereafter, for example, assume that there is an effect on the endoscope 2100 to rotate the tip portion 2130 due to a predetermined reason, and consequently, the endoscope image captured by the camera 2132 as illustrated in L22 is displayed on the display device 2900.

At this time, for example, the control device 2600 compares the image in L21 and the image in L22, and performs control for electrically driving the rolling of the tip portion 2130. With this control, the line-of-sight direction of the camera 2132 rotates within an x2-y2 plane so that the image captured by the camera 2132 is the image in L21. Note that FIG. 40 illustrates an example of maintaining the position of the tip portion 2130 using the rolling, the control device 2600 may further electrically control driving for advancing/retreating or bending in combination with the above-mentioned control.

Although the treatment tool 2400 is not illustrated in FIG. 40, the treatment tool 2400 may be inserted into the lumen as long as it is determined as NO in step S2200 in FIG. 39. In this case, the posture of the treatment tool 2400 may be changed with electric control for maintaining the position of the tip portion 2130. When the treatment tool 2400 has been inserted to a degree as illustrated in L11 in FIG. 38, it is a phase in which the treatment tool 2400 has not been inserted enough, and there is little effect on the treatment.

In this manner, the control device 2600 prioritizes maintaining of the position of the tip portion 2130 with respect to the opening of the lumen before the treatment tool 2400 is inserted enough into the opening of the lumen, but prioritizes maintaining of the posture of the treatment tool 2400 after the treatment tool 2400 is inserted enough into the opening of the lumen. Therefore, in the medical system 2010 in accordance with the present embodiment, until determining that the treatment tool 2400 is inserted into the lumen after the tip portion 2130 of the endoscope 2100 is positioned with respect to the opening of the lumen, the control device 2600 operates in the position maintaining mode to maintain the position of the tip portion 2130 of the endoscope 2100 with respect to the opening of the lumen (in a case of NO in step S2200, step S2400). In addition, after determining that the treatment tool 2400 is inserted into the lumen, the control device 2600 performs control for switching from the position maintaining mode to the treatment mode (in a case of YES in step S2200, step S2300). This enables structuring of the medical system 2010 that controls the tip portion 2130 of the endoscope 2100 in an appropriate operation mode depending on whether the treatment tool 2400 is inserted into the lumen.

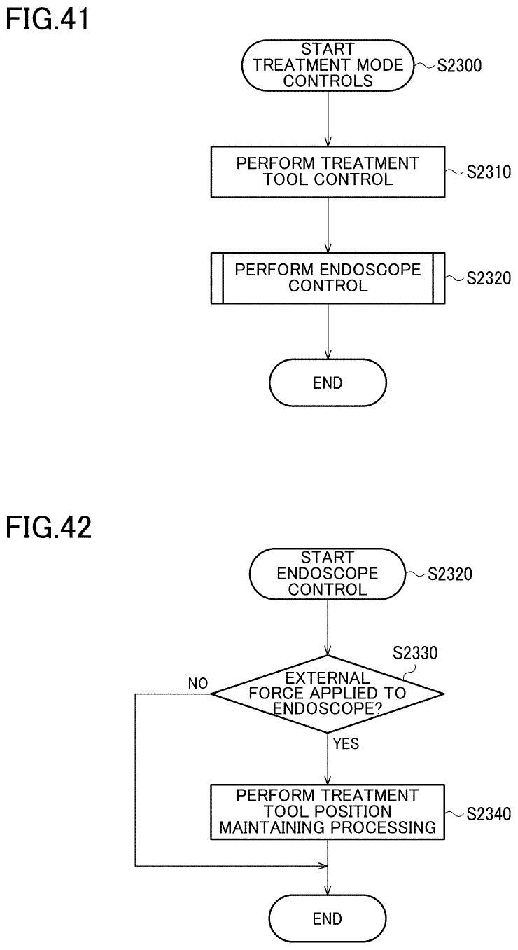

Details of the treatment mode control (step S2300) in FIG. 39 are now described with reference to a flowchart in FIG. 41. The control device 2600 performs treatment tool control (step S2310) and thereafter performs endoscope control (step S2320). The treatment tool control (step S2310) is processing of electrically controlling the treatment tool 2400 in treatment. Although not described in the flowchart, for example, in a case where the treatment tool 2400 is the basket treatment tool 1400-A for the purpose of extraction of the stone, the control device 2600 performs electric control including the following first processing, second processing, third processing, and fourth processing. The first processing is processing of moving the basket treatment tool 1400-A closer to the tip side than a position of the gallstone G to the tip side while observing the transmissive image. The second processing is processing of repeatedly performing an advancing/retreating operation, opening/closing operation, and rotation operation of the basket treatment tool 1400-A a predetermined number of times. The third processing is processing of determining whether the basket 1430 of the basket treatment tool 1400-A has fetched the gallstone G. The fourth processing is processing of pulling out the basket treatment tool 1400-A from the biliary duct in a state where the basket 1430 has fetched the gallstone G. Note that in a case where the basket 1430 has fetched the gallstone G in the third processing, the control device 2600 may perform the fourth processing, and in a case where the basket 1430 has not fetched the gallstone G in the third processing, the control device 2600 may perform the second processing again.

Additionally, in a case where the treatment tool 2400 is the basket treatment tool 1400-B for the purpose of crushing of the stone, the control device 2600 may additionally perform fifth processing of further crushing the gallstone G. The fifth processing can be implemented by, for example, the following method. For example, the control device 2600 performs processing of fetching the gallstone Gin the basket 1430 and partially contracting the basket 1430 to fix the gallstone G, and processing of advancing/retreating the second sheath 1420 to repeatedly collide a tip portion of the second sheath 1420 indicated in R in FIG. 37 against the gallstone G. Since the second sheath 1420 is made of a hard material, the gallstone G is crushed into small sizes by the fifth processing, and the basket treatment tool 1400-B including the gallstone G is easily pulled out from the biliary duct by the fourth processing.

The endoscope control (step S2320) is processing of electrically controlling the endoscope 2100 to maintain the position of the treatment tool 2400. For example, after the start of the treatment tool processing (step S2310), the control device 2600 electrically performs control for not accepting a signal input to the roll operating portion 2121, the motor unit 2816, and the coupling mechanism 2162. This can prevent displacement of the tip portion 2130 due to an erroneous operation or the like during execution of the treatment tool processing (step S2310). Accordingly, the position of the treatment tool 2400 that projects from the tip portion 2130 can be prevented from being changed by electric control. In this manner, in the medical system 2010 in accordance with the present embodiment, the control device 2600, in the treatment mode, electrically controls the treatment tool 2400 and also electrically controls the endoscope 2100 to maintain the position of the treatment tool 2400 in the lumen. This enables structuring of the medical system 2010 that performs electric control of the treatment tool 2400 and electric control of the endoscope 2100 in combination. This allows the posture of the treatment tool 2400 inserted into the lumen to be maintained. This allows the operator or the like to safely handle the treatment tool 2400 inserted into the lumen.

The endoscope control (step S2320) may be control of maintaining the position of the treatment tool 2400 when the endoscope 2100 is subjected to external force. Specifically, the endoscope control (step S2320) may be, for example, implemented as a processing example as indicated in a flowchart in FIG. 42. The control device 2600 performs processing of determining whether external force is applied to the endoscope 2100 (step S2330). When determining that external force is applied to the endoscope 2100 (YES in step S2330), the control device 2600 performs treatment tool position maintaining processing (step S2340), and ends the flow. In contrast, when determining that external force is not applied to the endoscope 2100 (NO in step S2330), the control device 2600 ends the flow.

The determination as to whether external force is applied to the endoscope 2100 can be implemented by various kinds of methods. For example, when receiving a signal having predetermined or higher intensity from the above-mentioned force sensor 2180 or the like, the control device 2600 may perform step S2330. When the position of the treatment tool 2400 is not within the predetermined range based on the transmissive image, the control device 2600 may determine YES in step S2330. In other words, when the position of the treatment tool 2400 is within the predetermined range based on the transmissive image even if the control device 2600 receives the signal from the force sensor 2180 or the like, the control device 2600 may determine NO in step S2330.

Alternatively, processing of periodically checking whether the position of the treatment tool 2400 is within the predetermined range based on the transmissive image may be processing in step S2330. When the position of the treatment tool 2400 is not within the predetermined range, the control device 2600 may equate this with external force being applied to the endoscope 2100, and determine YES in step S2330.

The treatment tool position maintaining processing (step S2340) is processing of, when the position of the treatment tool 2400 is outside the predetermined range, electrically controlling the endoscope 2100 so that the position of the treatment tool 2400 returns to a position within the predetermined range again. For example, when determining that external force is applied to the endoscope 2100 (YES in step S2330) at the time of electrically controlling the treatment using the basket treatment tool 1400, the control device 2600 controls a driving mechanism of the endoscope 2100, instead of controlling a driving mechanism of the operation wire or the like. In this manner, in the medical system 2010 in accordance with the present embodiment, when the tip portion 2130 of the endoscope 2100 is moved with respect to the opening of the lumen, the control device 2600 electrically controls the endoscope 2100 to maintain the position of the treatment tool 2400 in the lumen. Since the treatment tool 2400 projects from the tip portion 2130 of the endoscope 2100, the displacement of the endoscope 2100 due to a predetermined reason can change the posture of the treatment tool 2400. Hence, for example, there is a possibility that driving the operation wire in a situation where the basket treatment tool 1400 loses its posture applies an excessive load to the papillary opening or the like. In this respect, application of the method in accordance with the present embodiment enables structuring of the medical system 2010 that returns the posture of the treatment tool 2400 to a state before the movement of the tip portion 2130. Note that the predetermined cause is a disaster such as an earthquake, but may be, for example, a strong impact erroneously made by the operator or the like on any of constituent elements of the endoscope 2100 or the like.

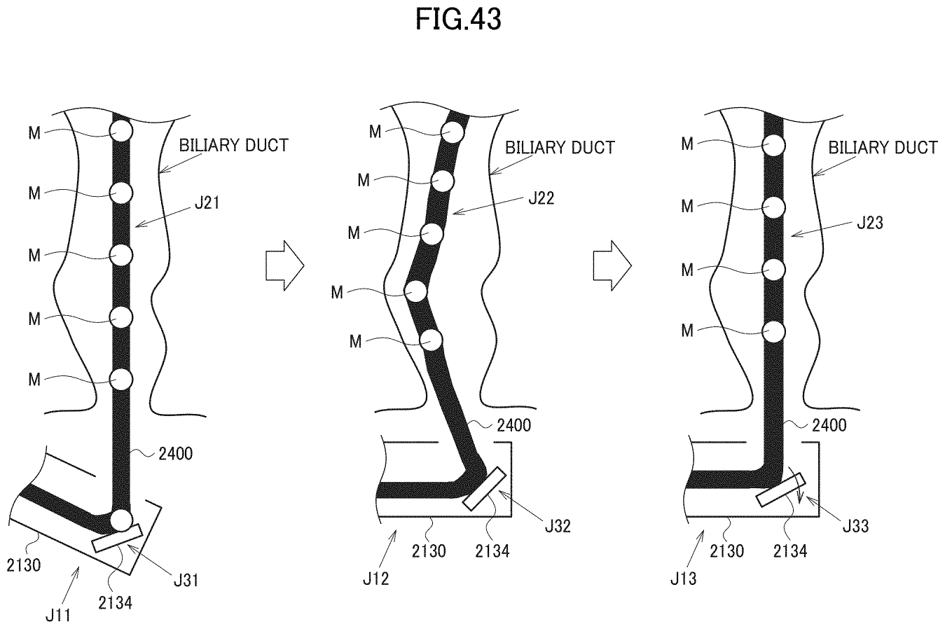

An application example of the treatment mode control (step S2300) is described with reference to FIG. 43. For example, assume that processing until the step of inserting the treatment tool 2400 into the lumen has been performed by the ERCP (step S2010) in FIG. 34. At this time, assume that the tip portion 2130 is fixed at a position indicated in J11, the treatment tool 2400 is inserted perpendicularly to the opening of the lumen, the shape of the treatment tool 2400 is a linear shape as indicated in J21, and the raising base 2134 is controlled at an angle as indicated in J31 with respect to the tip portion 2130. Note that FIG. 43 is a diagram conveniently illustrated, and the treatment tool 2400 is, for example, not required to have an exact linear shape. Since the markers M are added to the treatment tool 2400, the control device 2600 can recognize that a line that connects the markers M has a linear shape on the transmissive image displayed on the display device 2900.

Then assume that the tip portion 2130 is displaced to a position indicated in J12 by application of external force to the endoscope 2100 at a certain timing. At this time, since an angle of the raising base 2134 with respect to the tip portion 2130 is identical to the angle indicated in J31 as indicated in J32, the treatment tool 2400 becomes unable to be inserted perpendicularly to the opening of the lumen. As a result, as indicated in J22, the treatment tool 2400 comes in contact with the inner wall of the lumen and is folded, and the treatment tool 2400 becomes unable to maintain the linear shape.

The control device 2600 then determines YES in step S2330 in FIG. 42, and performs electric control for changing the angle of the raising base 2134 as indicated in J33 as the treatment tool position maintaining processing (step S2340). With this control, the treatment tool 2400 becomes to have the linear shape again as indicated in J23. In this manner, in the medical system 2010 in accordance with the present embodiment, the control device 2600 controls the raising base 2134 arranged in the tip portion 2130 of the endoscope 2100 to maintain the position of the treatment tool 2400 in the lumen. This enables structuring of the medical system 2010 that maintains the posture of the treatment tool 2400 inserted into the lumen in combination with control of the raising base 2134. This allows the posture of the treatment tool 2400 inserted into the lumen to be maintained in the situation where the position of the tip portion 2130 is changed when the treatment tool 2400 is inserted into the lumen. This allows the operator or the like to safely handle the treatment tool 2400 inserted into the lumen.

While FIG. 43 illustrates an example of controlling the raising base 2134 to maintain a substantially linear shape of the treatment tool 2400, the treatment tool position maintaining processing (step S2340) in accordance with the present embodiment is not limited thereto. For example, although not illustrated, the control device 2600 is also capable of performing control for changing a curve angle of the tip portion 2130 to maintain the substantially linear shape of the treatment tool 2400 in a situation in FIG. 43.

The control device 2600 is also capable of determining how to control the treatment tool position maintaining processing (step S2340) as appropriate depending on the change in shape of the treatment tool 2400. For example, although not illustrated, when determining that changing a roll rotation angle of the tip portion 2130 enables maintaining of the substantially linear shape of the treatment tool 2400, the control device 2600 may perform control for changing the roll rotation angle of the tip portion 2130. In this manner, in the medical system 2010 in accordance with the present embodiment, the control device 2600 electrically controls the roll rotation angle or curve angle of the tip portion 2130 of the endoscope 2100 to maintain the position of the treatment tool 2400 in the lumen. This enables structuring of the medical system 2010 that maintains the posture of the treatment tool 2400 inserted into the lumen in combination with electric control of the roll rotation angle or curve angle of the tip portion 2130. This allows the posture of the treatment tool 2400 inserted into the lumen to be maintained in the situation where the position of the tip portion 2130 is changed when the treatment tool 2400 is inserted into the lumen. This allows the operator or the like to safely handle the treatment tool 2400 inserted into the lumen.

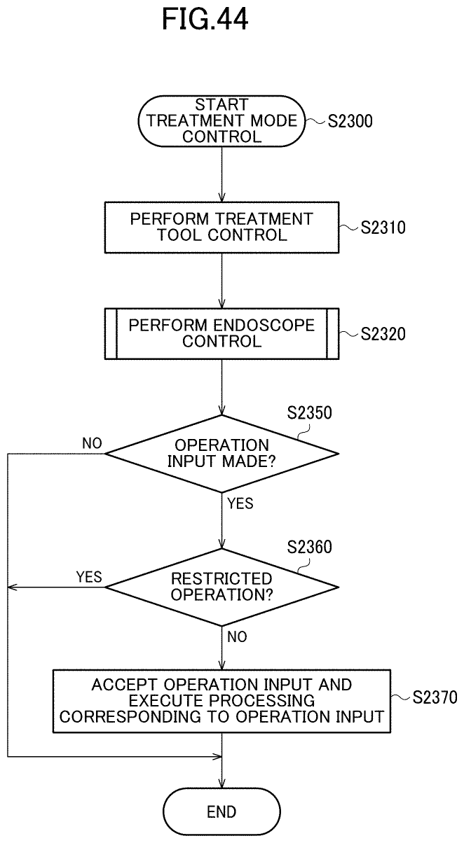

Note that the method in accordance with the present embodiment is not limited to the above-mentioned method, and can be modified in various manners. For example, the description has been given of the control for not accepting the operation input during execution of the treatment tool control (step S2310), but part of the operation input may be accepted. More specifically, for example, the treatment mode control (step S2300) in accordance with the present embodiment may be implemented as a processing example as indicated in a flowchart in FIG. 44. The flowchart in FIG. 44 is different from the flowchart in FIG. 40 in a point of further including step S2350, step S2360, and step S2370, which will be described later. Note that the endoscope control (step S2320) in FIG. 44 is similar to that in FIG. 41. Note that in FIG. 44, a description of overlapping processing is omitted.

The control device 2600 performs the endoscope processing (step S2320), and thereafter performs processing of determining whether the operation input has been made (step S2350). When determining that the operation input has been made (YES in step S2350), the control device 2600 performs processing of determining that an operation regarding the operation input is a restricted operation (step S2360). In contrast, when determining that no operation input has been made (NO in step S2350), the control device 2600 ends the flow.

When determining that the operation regarding the operation input is not the restricted operation (NO in step S2360), the control device 2600 accepts the operation input, and executes processing corresponding to the operation input (step S2370). In contrast, when determining that the operation regarding the operation input is the restricted operation (YES in step S2360), the control device 2600 ends the flow. In other words, when the operation regarding the operation input is the restricted operation, the control device 2600 performs processing of not accepting the operation input.

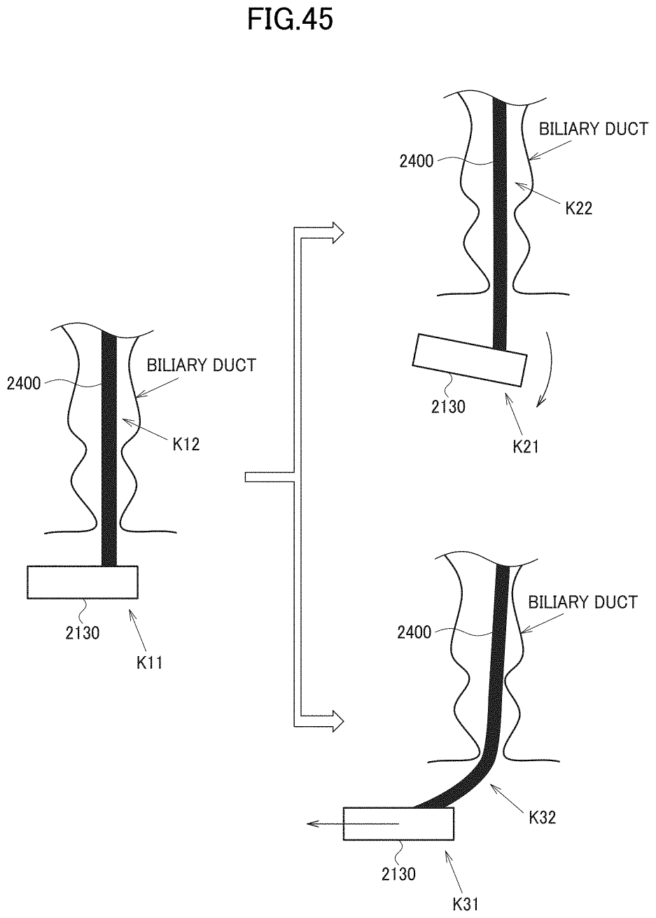

The operation to be restricted can be set as appropriate by the operator or the like. For example, in FIG. 45, assume that, in a situation where the tip portion 2130 is fixed at a position indicated in K11 and the treatment tool 2400 is inserted so as to maintain the substantially linear shape, serving as the predetermined shape, as indicated in K12, the operator operates the tip portion 2130 through the drive control device 2200. Note that the marker M or the like is not illustrated in FIG. 45. For example, when the tip portion 2130 pivots as indicated in K21, the substantially linear shape of the treatment tool 2400 remains to be maintained as indicated in K22. Since there is a low possibility that acceptance of such operation input affects the treatment, the operation to be restricted is set so that the determination in step S2360 in FIG. 44 is YES.

In contrast, for example, when the tip portion 2130 is retreated as indicated in K31 in FIG. 45, the shape of the treatment tool 2400 is significantly changed from the substantially linear shape as indicated in K32. In this manner, there is a high possibility that the operation of displacing the tip portion 2130 in the direction different from the insertion direction of the treatment tool 2400 affects the treatment. For example, when the above-mentioned basket treatment tool 1400 has fetched the calculus, there is a high possibility for applying a load to the vicinity of the papillary opening. In this regard, by application of the method in accordance with the present embodiment, when the operator performs an operation of displacing the tip portion 2130 in an undesirable direction, the determination as NO in step S2360 puts restriction on processing based on the operation, whereby the operator can safely perform the treatment.

As described above, known is the medical system that remotely performs electric control of the main body of the endoscope inserted into the body cavity and various types of treatment tools each inserted through the forceps channel. The specification of United States Patent Application Publication No. 2007/0185377 discloses the method of advancing/retreating and opening/closing the basket forceps in accordance with the embedded program. When the treatment tool 2400 is inserted into the lumen and the treatment is performed, the advancing/retreating drive control is desirably performed while the posture of the treatment tool 2400 is maintained because the vicinity of the papillary opening is narrow. Especially in a case where the basket treatment tool 1400 as the treatment tool 2400 is used, it is likely that retreating the basket treatment tool 1400 in a state where a calculus or the like having a large diameter has been fetched in the basket 1430 applies a high load to the vicinity of the papillary opening unless the posture of the basket treatment tool 1400 is maintained. The method of maintaining the posture of the treatment tool 2400 inserted into the lumen has not been proposed so far.

To address this, the medical system 2010 in accordance with the present embodiment includes the endoscope 2100 with the tip portion 2130 from which the treatment tool 2400 is projectable, and the control device 2600. After the tip portion 2130 of the endoscope 2100 is positioned with respect to the opening of the lumen and the treatment tool 2400 is inserted into the lumen, the control device 2600 operates in the treatment mode to maintain the position of the treatment tool 2400 in the lumen.

When a situation where the posture of the treatment tool 2400 in the lumen is not appropriate occurs, the present embodiment can bring the posture of the treatment tool 2400 into an appropriate state. This enables structuring of the medical system 2010 that appropriately handles the treatment tool 2400 inserted into the lumen. This allows the operator or the like to safely and appropriately perform treatment on the lumen using the endoscope 2100 and the treatment tool 2400.

Note that the operation in the treatment mode after the tip portion 2130 of the endoscope 2100 is positioned with respect to the opening of the lumen and the treatment tool 2400 is inserted into the lumen has been described in FIG. 34 and the like.

In the present embodiment, the control device 2600, in the treatment mode, may electrically control the treatment tool 2400 to maintain the position of the treatment tool 2400 in the lumen.

The present embodiment enables structuring of the medical system 2010 that electrically controls the treatment tool 2400 while maintaining the posture of the treatment tool 2400.

In the present embodiment, the control device 2600, in the treatment mode, may identify the position of the treatment tool 2400 based on the transmissive image including an image of the lumen, and electrically control the treatment tool 2400 to maintain the identified position of the treatment tool 2400 with respect to the lumen.

The present embodiment enables structuring of the medical system 2010 that electrically controls the treatment tool 2400, while visually grasping that the appropriate posture of the treatment tool 2400 is maintained in the lumen.

In the present embodiment, until determining that the tip portion 2130 of the endoscope 2100 is positioned with respect to the opening of the lumen and the treatment tool 2400 is inserted into the lumen, the control device 2600 may operate in the position maintaining mode to maintain the position of the tip portion 2130 of the endoscope 2100 with respect to the opening of the lumen. In addition, after determining that the treatment tool 2400 is inserted into the lumen, the control device 2600 may perform control for switching from the position maintaining mode to the treatment mode.

The present embodiment enables structuring of the medical system 2010 that controls the tip portion 2130 of the endoscope 2100 in an appropriate mode depending on whether the treatment tool 2400 is inserted into the lumen.

Note that the control for switching from the position maintaining mode to the treatment mode has been described with reference to FIG. 39 and the like.

In the present embodiment, the control device 2600, in the treatment mode, may electrically control the treatment tool 2400 and also electrically control the endoscope 2100 to maintain the position of the treatment tool 2400 in the lumen.

The present embodiment enables structuring of the medical system 2010 using the electric control of the treatment tool 2400 and the electric control of the endoscope 2100 in combination.

Note that the electric control of the treatment tool 2400 and the electric control of the endoscope 2100 being performed in combination has been described with reference to FIG. 41 and the like.

In the present embodiment, the treatment tool 2400 may be the treatment tool for removing the calculus, and the control device 2600 may perform control for removing the calculus using the treatment tool 2400 in the treatment mode.

The present embodiment enables structuring of the medical system 2010 that performs treatment for removing the calculus while maintaining the posture of the treatment tool 2400.

Note that the treatment tool for removing the calculus has been described with reference to FIGS. 33, 36, 37, and the like.

In the present embodiment, the control device 2600, in the treatment mode, may maintain the position of the treatment tool in the lumen based on the transmissive image including the image of the lumen.

The present embodiment enables structuring of the medical system 2010 that visually grasps that the appropriate posture of the treatment tool 2400 is maintained in the lumen.

Note that the transmissive image including the image of the lumen has been described with reference to FIG. 23 and the like.

In the present embodiment, the control device 2600 may identify the insertion path of the treatment tool 2400 based on the transmissive image, and electrically control the endoscope 2100 so that the shape of the insertion path has the predetermined shape.

The present embodiment enables structuring of the medical system 2010 that secures the appropriate insertion path of the treatment tool 2400 and that maintains the posture of the treatment tool 2400.

In the present embodiment, the treatment tool 2400 may include the plurality of markers M, and the control device 2600 may maintain the position of the treatment tool 2400 in the lumen based on the plurality of markers M seen in the transmissive image including the image of the lumen.

The present embodiment enables structuring of the medical system 2010 that more clearly grasps the posture of the treatment tool 2400 in the transmissive image.

Note that the marker M has been described with reference to FIGS. 36 and 37, and the like.

In accordance with the present embodiment, when the tip portion 2130 of the endoscope 2100 is moved with respect to the opening of the lumen, the control device 2600 may electrically control the endoscope 2100 to maintain the position of the treatment tool 2400 in the lumen.

The present embodiment enables structuring of the medical system 2010 that returns the posture of the treatment tool 2400 to the state before the movement of the tip portion 2130.

In the present embodiment, the control device 2600 may control the raising base 2134 arranged in the tip portion 2130 of the endoscope 2100 to maintain the position of the treatment tool 2400 in the lumen.

The present embodiment enables structuring of the medical system 2010 that maintains the posture of the treatment tool 2400 inserted into the lumen while using the control of the raising base 2134 in combination.

Note that the position of the treatment tool 2400 in the lumen being maintained by control of the raising base 2134 has been described with reference to FIG. 43 and the like.

In the present embodiment, the control device 2600 may electrically control the roll rotation angle or curve angle of the tip portion 2130 of the endoscope 2100 to maintain the position of the treatment tool 2400 in the lumen.

The present embodiment enables structuring of the medical system 2010 that maintains the posture of the treatment tool 2400 inserted into the lumen while using electric control of the roll rotation angle or curve angle of the tip portion 2130 in combination.

The present embodiment may be implemented as the operation method for the medical system 2010 as follows. That is, after the tip portion 2130 of the endoscope 2100, with the tip portion from which the treatment tool 2400 is projectable, is positioned with respect to the opening of the lumen and the treatment tool 2400 is inserted into the lumen, the operation method for the medical system 2010 includes the step of performing an operation in the treatment mode to maintain the position of the treatment tool 2400 in the lumen.

Note that in the operation method for the medical system 2010, the medical system 2010 plays a main role in performing each of the above-described steps.

Additionally, the present embodiment may be implemented as the non-transitory storage medium as follows. That is, the non-transitory information storage medium stores the program that causes the computer to execute the step of, after the tip portion 2130 of the endoscope 2100, with the tip portion 2130 from which the treatment tool 2400 is projectable, is positioned with respect to the opening of the lumen and the treatment tool 2400 is inserted into the lumen, performing the operation in the treatment mode to maintain the position of the treatment tool 2400 in the lumen.

Note that the medical system 2010 plays a main role in performing each step of the programs stored in the non-transitory information storage medium.

According to some aspects of the present embodiment, the following are provided.

1. A medical system comprising:

an endoscope comprising a tip portion from which a treatment tool is projectable; and

a controller comprising hardware, wherein the controller being configured to, after the tip portion of the endoscope is positioned with respect to an opening of a lumen and the treatment tool is inserted into the lumen, operate in a treatment mode to maintain a position of the treatment tool in the lumen.

2. The medical system of item 1, wherein, in the treatment mode, the controller is configured to identify the position of the treatment tool with respect to the opening of the lumen and electrically control the treatment tool to maintain the position of the treatment tool in the lumen.
3. The medical system of item 2, wherein, in the treatment mode, the controller is configured to identify the position of the treatment tool based on a transmissive image including an image of the lumen, and electrically control the treatment tool to maintain the identified position of the treatment tool with respect to the lumen.
4. The medical system as defined in item 1, wherein the controller is configured to:
operate in a position maintaining mode to maintain the position of the tip portion of the endoscope with respect to the opening of the lumen until determining that the treatment tool is inserted into the lumen after the tip portion of the endoscope is positioned with respect to the opening of the lumen, and
perform, after determining that the treatment tool is inserted into the lumen, control for switching from the position maintaining mode to the treatment mode.
   5. The medical system of item 1, wherein, in the treatment mode, the controller is configured to:

electrically control the treatment tool; and

electrically control the endoscope to maintain the position of the treatment tool in the lumen.

6. The medical system of item 1, wherein

the treatment tool is a treatment tool for removing a calculus, and

in the treatment mode, the controller is configured to control for removing the calculus using the treatment tool.

7. The medical system of item 1, wherein, in the treatment mode, the controller is configured to maintain the position of the treatment tool in the lumen based on a transmissive image including an image of the lumen.
8. The medical system of item 7, wherein the controller is configured to:

identify an insertion path of the treatment tool based on the transmissive image, and

electrically control the endoscope so that a shape of the insertion path becomes a predetermined shape.

9. The medical system of item 1, wherein

the treatment tool includes a plurality of markers, and

the controller is configured to maintain the position of the treatment tool in the lumen based on the plurality of markers seen in a transmissive image including an image of the lumen.

10. The medical system as defined in item 1, wherein, when the tip portion of the endoscope is moved with respect to the opening of the lumen, the controller is configured to electrically control the endoscope to maintain the position of the treatment tool in the lumen.
11. The medical system of item 1, wherein the controller is configured to control a raising base arranged in the tip portion of the endoscope to maintain the position of the treatment tool in the lumen.
12. The medical system of item 1, wherein the controller is configured to electrically control a roll rotation angle or a curve angle of the tip portion of the endoscope to maintain the position of the treatment tool in the lumen.
13. An operation method for a medical system, the method comprising performing an operation in a treatment mode, in which a tip portion of an endoscope is maintained in a position with respect to an opening of a lumen after the treatment tool is inserted into the lumen and the tip portion is projected from the lumen.
14. A non-transitory information storing medium that stores a program that causes a computer to execute a method, the method comprising performing an operation in a treatment mode, in which a tip portion of an endoscope is maintained in a position with respect to an opening of a lumen after the treatment tool is inserted into the lumen and tip portion is projected from the lumen.

Although the embodiments to which the present disclosure is applied and the modifications thereof have been described above, the present disclosure is not limited to the embodiments and the modifications thereof, and various modifications and variations in elements may be made in implementation without departing from the spirit and scope of the present disclosure. The plurality of elements disclosed in the embodiments and the modifications described above may be combined as appropriate to form various disclosures. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. A medical system comprising:
a medical instrument having an instrument motion during a procedure that is electrically driven, the instrument motion being at least one of forward and backward movement of an insertion section, a bending angle of a bending section of the insertion section, and rolling rotation of the insertion section;
an operation device configured to perform an operation input of the instrument motion; and
a processor comprising hardware, the processor being configured to:
control the electrically-driven instrument motion based on the operation input in a motion mode, the motion mode being one of a full auto mode to perform automatic control of the electrically-driven instrument motion and a semi auto mode in which an operator manually controls the instrument motion;
while the electrically-driven instrument motion is being controlled in the semi auto mode, interrupt the semi auto mode by automatic control of the electrically-driven instrument motion to perform intervention, and
subsequent to the intervention, switch to one of the full auto mode and the semi auto mode based on a determined use of the medical instrument.

2. The medical system of claim 1, wherein the processor is configured to:
switch the motion mode to the full auto mode when the medical instrument is being positioned, and
switch the motion mode to the semi auto mode when the medical instrument is being used for treatment after being positioned.

3. The medical system of claim 2, wherein the treatment is a cannulation using the medical instrument.

4. The medical system of claim 1, wherein
the medical instrument comprises an endoscope that electrically drives an endoscopic operation, which is the instrument motion, and captures an endoscope image, and
the processor is configured to perform the automatic control of positioning a distal end section of the endoscope based on the endoscope image in the full auto mode.

5. The medical system of claim 1, wherein when the medical instrument contacts an organ or tissue or when contact between the medical instrument and the organ or the tissue is expected in the semi auto mode, the processor is configured to perform the intervention by automatic control to avoid the contact.

6. The medical system of claim 1, wherein in the semi auto mode, when the medical instrument is moved to a route different from an insertion route in the procedure in which the semi auto mode is set, the processor performs the intervention by automatic control to restrict movement of the medical instrument to the route.

7. The medical system of claim 1, wherein
the processor includes, as the motion mode, a manual mode in which the intervention by automatic control is not performed, and
the processor switches between the full auto mode, the semi auto mode, and the manual mode in accordance with the procedure.

8. The medical system of claim 7, wherein the processor sets the motion mode to the manual mode when the medical instrument is determined to be inserted into a target.

9. The medical system of claim 7, wherein
in each step of the procedure, the medical system enables setting of correspondence as to which of the full auto mode, the semi auto mode, and the manual mode is set based on input information, and
the processor switches between the full auto mode, the semi auto mode, and the manual mode in accordance with the step of the procedure based on the correspondence.

10. The medical system of claim 1, wherein the procedure is endoscopic retrograde cholangiopancreatography.

11. The medical system of claim 10, wherein
the medical instrument includes an endoscope that electrically drives an endoscopic operation, which is the instrument motion, and
the processor is configured to:
switch the motion mode to the full auto mode when positioning the endoscope to a papillary portion of duodenum, and
switch the motion mode to a semi auto mode when inserting a treatment tool into a biliary duct.

12. The medical system of claim 10, wherein
the processor includes, as the motion mode, a manual mode in which the intervention by automatic control is not performed with respect to manual control, and
the processor is configured to switch between the full auto mode, the semi auto mode, and the manual mode in accordance with performance of an endoscopic retrograde cholangiopancreatography.

13. The medical system of claim 12, wherein
the medical instrument includes an endoscope that electrically drives an endoscopic operation, which is the instrument motion, and
the processor is configured to switch the motion mode to the manual mode when the endoscope is inserted into a papillary portion of duodenum.

14. The medical system of claim 1, wherein after the processor sets to the full auto mode, the processor is configured to ignore a manual instruction of the instrument motion from the operation device.

15. A cannulation method using an endoscope that is electrically driven during an endoscopic operation, a motion of the endoscope being at least one of forward and backward movement of an insertion section, a bending angle of a bending section of the insertion section, and rolling rotation of the insertion section, the cannulation method comprising:
switching to a full auto mode in which automatic control of the electrically-driven endoscopic operation is performed to control the motion of the endoscope to position the endoscope to a papillary portion of duodenum;
switching to a semi auto mode in which an operator manually controls the motion of the endoscope;
while the motion of the endoscope is being controlled in the semi auto mode, interrupt the semi auto mode by automatic control of the motion of the endoscope to perform intervention, and
subsequent to the intervention, switch to one of the full auto mode and the semi auto mode based on a determined use of the endoscope.

16. A control apparatus for a medical system comprising:
a processor comprising hardware, the processor being configured to:
control an electrically-driven instrument motion of a medical instrument based on an operation input in a motion mode, the motion mode being one of a full auto mode to perform automatic control of the electrically-driven instrument motion and a semi auto mode in which an operator manually controls the instrument motion;
when controlling the electrically-driven instrument motion in the semi auto mode, interrupt the semi auto mode by automatic control of the electrically-driven instrument motion to perform intervention, and
subsequent to the intervention, switch to one of the full auto mode and the semi auto mode based on a determined use of the medical instrument.
